(19) United States
(12) Patent Application Publication
(10) Pub. No.: US 2021/0207099 A1
(43) Pub. Date: Jul. 8, 2021

(54) METHODS AND COMPOSITIONS FOR GENERATING CELLS OF ENDODERMAL LINEAGE AND BETA CELLS AND USES THEREOF

(71) Applicant: Washington University, St. Louis, MO (US)

(72) Inventors: Jeffrey R. Millman, St. Louis, MO (US); Nathaniel Hogrebe, St. Louis, MO (US); Jiwon Song, St. Louis, MO (US); Leonardo Velazco-Cruz, St. Louis, MO (US)

(21) Appl. No.: 17/055,946

(22) PCT Filed: May 16, 2019
(86) PCT No.: PCT/US2019/032643

§ 371 Date: Nov. 16, 2020

Related U.S. Application Data
(60) Provisional application No. 62/672,300, filed on May 16, 2018.
(60) Provisional application No. 62/672,695, filed on May 17, 2018.
(60) Provisional application No. 62/799,252, filed on Jan. 31, 2019.
(60) Provisional application No. 62/789,724, filed on Jan. 8, 2019.

(51) Int. Cl. C12N 5/071

(52) U.S. Cl. CPC C12N 5/0676; C12N 2506/45; C12N 2501/16; C12N 2501/395; C12N 2501/727; C12N 2501/999; C12N 2506/02; C12N 2501/117

(57) ABSTRACT

Among the various aspects of the present disclosure is the provision of methods and compositions for the generation of cells of endodermal lineage and beta cells and uses thereof.

### CROSS-REFERENCE TO RELATED APPLICATIONS

This application claims priority from U.S. Provisional Application Ser. No. 62/672,300 filed on 16 May 2018; U.S. Provisional Application Ser. No. 62/672,695 filed on 17 May 2018; U.S. Provisional Application Ser. No. 62/799,252 filed on 31 Jan. 2019; and U.S. Provisional Application Ser. No. 62/789,724 filed on 8 Jan. 2019, which are incorporated herein by reference in their entireties.

### STATEMENT REGARDING FEDERALLY SPONSORED RESEARCH OR DEVELOPMENT

This invention was made with government support under grant number DK114233 awarded by National Institutes of Health. The government has certain rights in the invention.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present disclosure generally relates to cellular therapies and methods of making beta-like cells.

### SUMMARY OF THE INVENTION

Among the various aspects of the present disclosure is the provision of methods and compositions to generate cells of endodermal lineage and uses thereof.

An aspect of the present disclosure provides for a method of generating insulin-producing beta cells in a suspension comprising: providing a stem cell; providing serum-free media; contacting the stem cell with a TGFβ/Activin agonist or a glycogen synthase kinase 3 (GSK) inhibitor or WNT agonist for an amount of time sufficient to form a definitive endoderm cell; contacting the definitive endoderm cell with a FGFR2b agonist for an amount of time sufficient to form a primitive gut tube cell; contacting the primitive gut tube cell with an RAR agonist, and optionally a rho kinase inhibitor, a smoothened antagonist, a FGFR2b agonist, a protein kinase C activator, or a BMP type 1 receptor inhibitor for an amount of time sufficient to form an early pancreas progenitor cell; incubating the early pancreas progenitor cell for at least about 3 days and optionally contacting the early pancreas progenitor cell with a rho kinase inhibitor, a TGF-β/Activin agonist, a smoothened antagonist, an FGFR2b agonist, or a RAR agonist for an amount of time sufficient to form a pancreatic progenitor cell; contacting the pancreatic progenitor cell with an Alk5 inhibitor, a gamma secretase inhibitor, SANT1, Erbb1 (EGFR) or Erbb4 agonist, or a RAR agonist for an amount of time sufficient to form an endoderm cell; or resizing cell clusters within about 24 hours and allowing the endoderm cell to mature for an amount of time in serum-free media sufficient to form a beta cell.

In some embodiments, the TGFβ/Activin agonist is Activin A; the glycogen synthase kinase 3 (GSK) inhibitor or the WNT agonist is CHIR; the FGFR2b agonist is KGF; the smoothened antagonist is SANT-1; the RAR agonist is retinoic acid (RA); the protein kinase C activator is PdBU; the BMP type 1 receptor inhibitor is LDN; the rho kinase inhibitor is Y27632; the Alk5 inhibitor is Alk5i, or the Erbb4 agonist is betacellulin.

In some embodiments, the serum-free media comprises one or more selected from the group consisting of: MCDB131, glucose, NaHCO₃, BSA, ITS-X, Glutamax, vitamin C, penicillin-streptomycin, CMRL 10666, FBS, Heparin, NEAA, trace elements A, trace elements B, or ZnSO₄.

In some embodiments, the method comprises reducing cluster size of the endoderm, wherein resizing cell clusters comprise breaking apart clusters and reaggregating prior to maturation into beta cells.

In some embodiments, the pancreatic progenitor cell is not incubated with any one or more of serum, T3, N-acetyl cysteine, Trolox, and R428.

In some embodiments, the amount of time sufficient to form a definitive endoderm cell, a primitive gut tube cell, an early pancreas progenitor cell, a pancreatic progenitor cell, an endoderm cell, or a beta cell is between about 1 day and about 8 days.

In some embodiments, the method does not comprise the use of a TGFβR1 inhibitor (e.g., Alk5 inhibitor II) in the maturation of endoderm cells to beta cells.

In some embodiments, the absence of a TGFβR1 inhibitor allows for TGFβ signaling and promotes functional maturation of beta cells from endoderm cells.

In some embodiments, the absence of TGFβR1 inhibitor allows for an increase in insulin secretion from the cells in response to an increased glucose level or an increased secretogouge level.

In some embodiments, the method does not comprise T3, N-acetyl cysteine, Trolox, or R428 in the maturation of endoderm cells to beta cells.

In some embodiments, the beta cell is an SC-β cell expressing at least one β cell marker and undergoes glucose-stimulated insulin secretion (GSIS) comprising first and second phase dynamic insulin secretion; the beta cell secretes insulin in substantially similar amounts compared to cadaveric human islets; or the beta cell retains functionality for 1 or more days.

In some embodiments, the stem cell is an HUES8 embryonic cell, SEVA 1016, or SEVA 1019.

Another aspect of the present disclosure provides for a method of treating a subject in need thereof comprising: administering a therapeutically effective amount of insulin-producing beta cells to a subject, wherein the beta cells are generated according to the above.

Another aspect of the present disclosure provides for a method of differentiating a stem cell into a cell of endodermal lineage comprising: providing a stem cell; providing serum-free media; contacting the stem cell with a TGFβ/Activin agonist and a glycogen synthase kinase 3 (GSK) inhibitor or WNT agonist for an amount of time sufficient to form a definitive endoderm cell; contacting the definitive endoderm cell with a FGFR2b agonist for an amount of time sufficient to form a primitive gut tube cell; contacting the primitive gut tube cell with an RAR agonist and, optionally, a smoothened antagonist/sonic hedgehog inhibitor, a FGF family member/FGFR2b agonist, a protein kinase 3 activator, a BMP inhibitor, or a rho kinase inhibitor, optionally, for an amount of time sufficient to form an early pancreas progenitor cell; incubating the early pancreas progenitor cell for at least about 3 days and optionally comprising contacting the early pancreas progenitor cell with a smoothened antagonist, an FGFR2b agonist, a RAR agonist, a rho kinase inhibitor, or a TGF-β/Activin agonist, for an amount of time sufficient to form a pancreatic progenitor cell; contacting the pancreatic progenitor cell with an Alk5 inhibitor/TGF-β receptor inhibitor, thyroid hormone, and a gamma secretase inhibitor and optionally SANT1, a Erbb1 (EGFR) or Erbb4 agonist/EGF family member, or a RAR agonist for an amount of time sufficient to form an endodermal cell or endocrine cell; optionally contacting the endodermal cell or the endocrine cell with an Alk5 inhibitor/TGF-β receptor inhibitor or a thyroid hormone for an amount of time sufficient to form a cell of endodermal lineage (e.g., pancreatic cell, liver cell, or beta cell/SC-β cell); or plating cells on a stiff or soft substrate or introducing a cytoskeletal-modulating agent to cells, optionally the cytoskeletal-modulating agent comprises latrunculin A, latrunculin B, nocodazole, cytochalasin D, jasplakinolide, blebbistatin, y-27632, y-15, gdc-0994, or an integrin modulating agent, at a time and for an amount of time sufficient to increase differentiation efficiency.

Another aspect of the present disclosure provides for a method of differentiating a stem cell into a cell of endodermal lineage comprising: incubating a stem cell in media comprising a TGFβ/Activin agonist, Activin A, a WNT agonist, and CHIR for about 24 hours, followed by about 3 days of incubating cells in media comprising the Activin A absent CHIR, resulting in stage 1, definitive endoderm cells; generating exocrine pancreas cells comprising incubating the stage 1, definitive endoderm cells for about two days in media comprising a FGFR2b agonist, KGF, resulting in stage 2 cells; incubating the stage 2 cells for 2 days in media comprising the FGFR2b agonist, KGF; a BMP inhibitor, LDN193189, TPPB; a RAR agonist, retinoic acid (RA); and a smoothened antagonist, SANT1, resulting in stage 3 cells; incubating stage 3 cells for about four days in media comprising the FGFR2b agonist, KGF; the BMP inhibitor, LDN193189; TPPB, the RAR agonist, retinoic acid; and the smoothened antagonist, SANT1, resulting in stage 4 cells, wherein latrunculin A is added for about the first 24 hours of incubation or nocodazole is added for an entirety of about four days of incubation; and incubating stage 4 cells in media comprising bFGF for about six days, wherein nicotinamide is added during the last two days of the six days; generating intestine cells comprising incubating the stage 1, definitive endoderm cells for about four days in media comprising the WNT agonist, CHIR and FGF4, wherein latrunculin A is added for about the first 24 hours of incubation or nocodazole is added for the entirety of about four days of incubation, resulting in stage 2 cells; incubating stage 2 cells for about 7 days in media comprising R-spondin1 and the BMP inhibitor, LDN193189; or generating liver cells comprising incubating the stage 1, definitive endoderm cells for about two days in media comprising the FGFR2b agonist, KGF, resulting in stage 3 cells; incubating stage 3 cells for about four days in media comprising BMP4, wherein the RAR agonist, retinoic acid and either latrunculin A or nocodazole were added for about the first 24 hours of incubation, resulting in stage 4 cells; and incubating the stage 4 cells in media comprising OSM, HGF, and dexamethasone for about 5 days.

In some embodiments, the methods comprise resizing clusters prior to forming a cell of endodermal lineage.

In some embodiments, the TGFβ/Activin agonist is Activin A; the glycogen synthase kinase 3 (GSK) inhibitor or the WNT agonist is CHIR; the FGFR2b agonist is KGF; the smoothened antagonist or sonic hedgehog inhibitor is SANT-1; the FGF family member/FGFR2b agonist is KGF; the RAR agonist is RA; the protein kinase 3 activator is PDBU; the BMP inhibitor is LDN; the rho kinase inhibitor is Y27632; the Alk5 inhibitor/TGF-β receptor inhibitor is Alk5i, the thyroid hormone is T3; the gamma secretase inhibitor is XXI; the Erbb1 (EGFR) or Erbb4 agonist/EGF family member is betacellulin; or RAR agonist is RA.

In some embodiments, the serum-free media comprises one or more selected from the group consisting of: MCDB131, glucose, NaHCO₃, BSA, ITS-X, Glutamax, vitamin C, penicillin-streptomycin, CMRL 10666, FBS, Heparin, NEAA, trace elements A, trace elements B, or ZnSO₄.

In some embodiments, the amount of time sufficient to form a definitive endoderm cell, a primitive gut tube cell, an early pancreas progenitor cell, a pancreatic progenitor cell, an endoderm cell, or a beta cell is between about 1 day and about 15 days.

In some embodiments, the early pancreatic progenitor cells are plated or YAP activated with s1p (sphingosine-1-phosphate) (e.g., during about stage 4), to increase SC-β cell induction, prevent undesirable premature endocrine commitment, or allowing for correct timing of transcription factor expression.

In some embodiments, Latrunculin A, Latrunculin B, or nocodazole is introduced (e.g., throughout stage 4, at stage 5 or about day 7) to the pancreatic progenitor cell, resulting in enhanced endocrine induction of plated cells and enhanced glucose-stimulated insulin secretion of subsequently generated β cells.

In some embodiments, Latrunculin A or Latrunculin B is introduced to the pancreatic progenitor cell, generating a cell of endodermal lineages, such as liver cells, or the Latrunculin A or Latrunculin B disrupts cytoskeleton actin (e.g., introduction of Latrunculin A or Latrunculin B prior to stage 5 results in liver cells or introduction of Latrunculin A or Latrunculin B throughout stage 5 results in increased number of β cells).

In some embodiments, a YAP inhibitor (e.g., Verteporfin) is introduced to the pancreatic progenitor cell.

In some embodiments, Latrunculin A or Latrunculin B is introduced to the pancreatic progenitor cell, increasing glucose-mediated insulin secretion or insulin gene expression.

In some embodiments, the cell of endodermal lineage is selected from a beta cell, a liver cell, or a pancreas cell.

In some embodiments, the method enhances induction and function of beta cells.

In some embodiments, the method is comprises culturing in a planar (attached) culture.

In some embodiments, the method comprises plating cells on a stiff substrate, wherein NKX6.1 expression increases on a stiff substrate compared to NKX6.1 expression on a soft substrate or in a suspension culture.

In some embodiments, planar (attached) cells are dispersed and reaggregated or combined with surfaces that change hydrophobicity with an external cue (e.g., temperature), allowing detachment of cells and retaining cell arrangement, extracellular matrix proteins, and insulin secretion.

In some embodiments, the beta cells are SC-β cells.

In some embodiments, the stem cells are selected from HUES8 and 1016SeVA.

Another aspect of the present disclosure provides for a method of screening comprising: providing a cell generated from any one of the above aspects or embodiments; or introducing a compound or composition to the cell.

Another aspect of the present disclosure provides for a method of treating a subject in need thereof comprising: administering a therapeutically effective amount of cells of endodermal lineage to a subject, wherein the cells are generated according to any one of the above aspects or embodiments.

In some embodiments, the subject has diabetes or the cells are transplanted into the subject.

Another aspect of the present disclosure provides for a cell generated by the method of any one of the above aspects or embodiments.

Another aspect of the present disclosure provides for methods for generating or a cell generated by the method of any one of the above aspects or embodiments, wherein the cell of endodermal lineage, beta cell, or intermediate cell expresses CDX2, CHGA, FOXA2, SOX17, PDX1, NKX6-1, NGN3, NEUROG3, NEUROD1, NXK2-2, ISL1, KRT7, KRT19, PRSS1, PRSS2, or INS.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1F show SC-β cell clusters undergo glucose-stimulated insulin secretion (GSIS). (A) Overview of differentiation procedure used. (B) Images of unstained whole Stage 6 clusters under phase contrast (top) or stained with dithizone (DTZ) imaged under bright field (bottom). (C) Immunostaining of sectioned paraffin-embedded Stage 6 clusters stained for Glucagon (GCG), NKX6-1, or PDX1 in red, C-peptide (CP) in green, and stained with the nuclei marker 4,6-diamidino-2-phenylindole (DAPI). (D) Human insulin secretion of Stage 6 cells generated with the protocol from this study (n=16), Stage 6 cells generated with the Pagliuca protocol (n=12), and cadaveric human islets (n=12) in a static glucose-stimulated insulin secretion (GSIS) assay. **P<0.01, ****P<0.001 by one-sided paired t-test. #P<0.05, ####P<0.0001 by one-way ANOVA Dunnett multiple comparison test comparing to this study. (E) Static GSIS assay of Stage 6 cells from this study subjected to either 2, 5.6, 11.1, or 20 mM glucose (n=4). *P<0.05, ***P<0.001, not significant (ns) by one-way ANOVA Dunnett multiple comparison test comparing to 2 mM glucose. (F) Dynamic human insulin secretion of Stage 6 cells generated with the protocol from this study (n=12), Stage 6 cells generated with the Pagliuca protocol (n=4), and cadaveric human islets (n=12) in a perfusion GSIS assay. Cells are perfused with low glucose (2 mM) except where high glucose (20 mM) is indicated. Act A, activin A; CHIR, CHIR9901, KGF, keratinocyte growth factor; RA, retinoic acid; Y, Y27632; LDN, LDN193189; PdbU, phorbol 12,13-dibutyrate; T3, triiodothyronine; Alk5i, Alk5 inhibitor type ESFM, Enriched Serum-Free Medium. All Stage 6 data shown is with HUES8.

FIG. 2A-FIG. 2D show SC-β cells express β cell and islet markers. (A) Immunostaining of Stage 6 clusters single-cell dispersed, plated overnight, and stained for Chromogranin A (CHGA), GCG, Somatostatin (SST), NEUROD1, NKX6-1, PDX1, or PAX6 in red, C-peptide (CP) in green, and stained with DAPI. (B) Representative flow cytometric dot plots of Stage 6 clusters single-cell dispersed and immunostained for the indicated markers. (C) Box-and-whiskers plots quantifying fraction of cells expressing the indicated markers. Each point is an independent experiment. (D) Real-time PCR analysis of Stage 6 cells generated with the protocol from this study (n=8), Stage 6 cells generated with the Pagliuca protocol (n=5), and cadaveric human islets (n=7). ns, *P<0.05, **P<0.01, ***P<0.001, ****P<0.0001 by one-way ANOVA Dunnett multiple comparison test comparing to this study. All Stage 6 data shown is with HUES8.

FIG. 3A-FIG. 3H show SC-β cells greatly improve glucose tolerance and have persistent function for months after transplantation. (A) Serum human insulin of a non-STZ-treated mouse cohort (n=3) 6 months after transplantation fasted overnight 0 and 60 min after an injection of 2 g/kg glucose. **P<0.01 by one-sided paired t-test. (B) Immunostaining of sectioned paraffin-embedded explanted kidneys of non-STZ-treated mice 6 months after transplantation for C-peptide with DAPI (left) or C-peptide and PDX1 with DAPI (right). White dashed line is manually drawn to show border between kidney and graft (*). (C) Glucose tolerance test (GTT) 10 d after surgery for STZ-treated mice cohort without a transplant (STZ, No Txp; n=6), untreated mice without a transplant (No STZ, No Txp; n=5), and STZ-treated mice with a transplant (STZ, Txp; n=6). *P<0.05, **P<0.01, ***P<0.001, ****P<0.0001 by two-way ANOVA Tukey multiple comparison. (D) Area under the curve (AUC) calculations for data shown in (C). **P<0.01 by one-way ANOVA Tukey multiple comparison test. (E) Serum human insulin of STZ, Txp mice (n=5) fasted overnight 0 and 60 min after an injection of 2 g/kg glucose. **P<0.01 by one-sided paired t-test. (F) GTT 10 wk after surgery for STZ, No Txp mice (n=6), No STZ, No Txp mice (n=4), and STZ, Txp mice (n=5). **P<0.01, ***P<0.001, ****P<0.0001 by two-way ANOVA Tukey multiple comparison test. (G) AUC calculations for data shown in (d). **P<0.01 by one-way ANOVA Tukey multiple comparison test. (H) Serum human insulin of STZ, Txp mice (n=5) fasted overnight 0 and 60 min after an injection of 2 g/kg glucose. **P<0.01 by one-sided paired t-test. All data shown is with HUES8. Panels (A-B) are SCID/Beige and panels (C-H) are NOD/SCID mice.

FIG. 4A-FIG. 4C show SC-β cells have transient dynamic function in vitro, respond to multiple stimuli, and sustain second phase insulin secretion at high glucose. (A) Dynamic human insulin secretion cells in Stage 6 for 5, 9, 15, 22, 26, and 35 d in a perfusion GSIS assay. Data for each individual time point is shown as mean±SEM and the final graph shows only the means of each graph. Cells are perfused with low glucose (2 mM) except where high glucose (20 mM) is indicated (n=3 for each Stage 6 time point). (B) Dynamic human insulin secretion of Stage 6 cells in a perfusion GSIS assay treated with multiple secretagogues. Cells are perfused with low glucose (2 mM) except where high (20 mM) glucose is indicated (Glu), then perfused with a second challenge of high glucose alone or with additional compounds (Tolbutamide, IBMX, and Extendin-4 on the left; KCL and L-Arginine on the right) where indicated (Glu+Factor). (C) Dynamic human insulin secretion of Stage 6 cells in a perfusion GSIS assay with an extended high glucose treatment. Cells are perfused with low glucose (2 mM) except where high glucose (20 mM) is indicated (n=3). All data shown is with HUES8.

FIG. 5A-FIG. 5F shows Alk5 inhibitor type II reduces SC-β cell GSIS. (A) Box-and-whiskers plot of human insulin secretion of Stage 6 cells in static GSIS assay treated with DMSO or Alk5i (n=9). ***P<0.001, ****P<0.0001 by two-way paired t-test; ####P<0.0001 by two-way unpaired t-test. (B) Cellular insulin content of Stage 6 cells treated with DMSO or Alk5i (n=18). ****P<0.0001 by two-way unpaired t-test. (C) Cellular proinsulin/insulin content ratio of Stage 6 cells treated with DMSO or Alk5i (n=17). ns by two-way unpaired t-test. (D-E) Representative flow cytometric dot plots of Stage 6 clusters single-cell dispersed and immunostained for Chromogranin A and PDX1 (D) or C-peptide and NKX6-1 (E). (F) Dynamic human insulin secretion of Stage 6 cells treated with DMSO or Alk5i in a perfusion GSIS assay. Cells are perfused with low glucose (2 mM) except where high glucose (20 mM) is indicated (n=12). All data shown is with HUES8.

FIG. 6A-FIG. 6E shows blocking TGFβ signaling during Stage 6 hampers GSIS. (A) Western blot of Stage 6 cells cultured with DMSO or Alk5i stained for phosphorylated SMAD ⅔ (pSMAD⅔), total SMAD ⅔ (tSMAD⅔), and Actin. Data shown is from HUES8. (B) Real-time PCR of Stage 6 cells transduced with lentiviruses containing shRNA against GFP (control) or one of two sequences against TGFBR1 (TGFBR1 #1 and #2) (n=3). ****P<0.0001 by one-way ANOVA Dunnett multiple comparison test comparing to GFP. (C) Western blot of Stage 6 cells transduced with lentiviruses containing GFP or TGFBR1 #1 shRNA. Data shown is from 1013-4FA. (D) Human insulin secretion of Stage 6 cells in static GSIS assay transduced with lentiviruses containing GFP, TGFBR1 #1, or TGFBR1 #2 shRNA (n=3). **P<0.01 by paired two-way t-test. ##P<0.01 by one-way ANOVA Dunnett multiple comparison test comparing to GFP. Data shown is from HUES8. (E) Dynamic human insulin secretion of Stage 6 cells transduced with lentiviruses containing GFP or TGFBR1 #1 shRNA in a perfusion GSIS assay. Cells are perfused with low glucose (2 mM) except where high glucose (20 mM) is indicated (n=4). Data shown is from HUES8.

FIG. 7A-FIG. 7G shows Alk5 inhibitor type II treatment during Stage 5 is important for generation of insulin-producing cells. (A-B) Representative flow cytometric dot plots of Stage 5 clusters single-cell dispersed and immunostained for Chromogranin A and NKX6-1 (A) or C-peptide and NKX6-1 (B). (C) Fraction of cells expressing the indicated markers (n=4 except CHGA, which was n=3). *P<0.05, **P<0.01, or ns by unpaired two-way t-test. (D-F) Real-time PCR measuring relative gene expression of Stage 5 cells cultured with DMSO or Alk5i for pancreatic hormones (D), β cell markers (E), or endocrine markers (F) (n=6). *P<0.05, **P<0.01, ****P<0.0001, or ns by unpaired two-way t-test. (G) Human insulin secretion at 20 mM glucose of cells cultured in Stage 5 in either DMSO or Alk5i plus an additional 7 d in Stage 6 without Alk5i and without cluster resizing (n=3). **P<0.01 by unpaired two-way t-test. All data shown is from HUES8.

FIG. 8A-FIG. 8D shows data leading to new differentiation strategy and hiPSC reproduction. (A) Human insulin secretion of Stage 6 cells generated in CMRLS or ESFM, with or without resizing, and with or without factors (Alk5i and T3) in a static GSIS assay. The combinations investigated were (1) CMRLS, no resize, no factors (n=3), (2) CMRLS, yes resize, no factors (n=6), (3) ESFM, no resize, no factors (n=3), (4) ESFM, yes resize, no factors (n=3), (5) ESFM, yes resize, yes factors (n=3). HUES8 cell line used. (B) Flow cytometric dot plots of Stage 6 cells generated in CMRLS or ESFM, with or without resizing, and with or without factors (Alk5i and T3) immunostained for C-peptide and NKX6-1. HUES8 cell line used. (C) Human insulin secretion in a static GSIS assay of three hiPSC lines (n=3 each). *P<0.05, **P<0.01, and ***P<0.0001 by one-sided paired t-test. (D) Dynamic human insulin secretion of Stage 6 cells generated with two hiPSC lines in a perfusion GSIS assay. Cells are perfused with low glucose (2 mM) except where high glucose (20 mM) is indicated (n=3 for 1013-4FA and n=4 for 1016SeVA).

FIG. 9A-FIG. 9C shows additional immunostaining data for Stage 6 cells. (A) Immunostaining of Stage 6 clusters single-cell dispersed, plated overnight, and stained for the indicated markers. Stage 6 cells were generated from two hiPSC lines with the protocol from this paper and the HUES8 cell line with the Pagliuca protocol. Scale bar=50 μm for 1016SeVA and 1013-4FA and 25 μm for Pagliuca protocol. (B-C) Flow cytometric dot plots of Stage 6 cells generated from two hiPSC lines with the protocol from this paper and the HUES8 cell line with the Pagliuca protocol stained with the indicated markers.

FIG. 10 shows additional gene expression data for Stage 6 cells. Gene expression data for Stage 6 cells generated with the new differentiation protocol from the HUES8 (n=8) and 1013-4FA (n=10) lines and human islets (n=7) measured with real-time PCR. The HUES8 and human islet plotted here is the same as from FIG. 2.

FIG. 11A-FIG. 11D shows additional immunostaining, serum human insulin measurements, and mouse C-peptide measurements. (A) Immunostaining of sectioned paraffin-embedded explanted kidneys of non-STZ-treated mice 6 months after transplantation for C-peptide (CP; β cell marker), PDX1 ((3 cell marker), glucagon (GCG; a cell marker), somatostatin (SST; δ cell marker), KRT19 (ductal marker), and trypsin (acinar marker). Scale bar=25 μm. (B) Serum human insulin of STZ, No Txp mice (n=6) and No Stz, No Txp (n=5) fasted overnight 0 and 60 min after an injection of 2 g/kg glucose. (B) Serum mouse C-peptide of STZ, No Txp (n=6), No STZ, No Txp (n=4), and STZ, TXP (n=5). ****P<0.0001 and ns by one-way ANOVA Tukey multiple comparison test. (C) Immunostaining of sectioned paraffin-embedded explanted kidneys of STZ-treated mice 11 wk after transplantation for the indicated markers. Scale bar=25 μm. HUES8 cell line used.

FIG. 12A-FIG. 12B shows temporal flow cytometry during Stage 6 and KCl challenge of human islets. (A) Flow cytometric dot plots of Stage 6 cells at early (9 d) and late (26 d) time points stained for C-peptide and NKX6-1. HUES8 cell line used. (B) Dynamic human insulin secretion of human islets in a perfusion GSIS assay perfused with low glucose (2 mM) except where high (20 mM) glucose is indicated (Glu), then perfused with a second challenge of high glucose with KCl where indicated (Glu+Factor) (n=4).

FIG. 13A-FIG. 13C shows stage 6 cells generated from hiPSC undergo GSIS that is inhibited by Alk5i, flow cytometry controls, and gene expression data. (A) Human insulin secretion of Stage 6 cells generated from three hiPSC lines (1013-4FA, n=4; 1016SeVA, n=3; 1019SeVF, n=3) in static GSIS assay treated with DMSO or Alk5i. *P<0.05, **P<0.01, ****P<0.0001 by two-way paired t-test; ##P<0.01, ###P<0.001, ####P<0.0001 by two-way unpaired t-test. The control data here is the same data in FIG. 21. (B) Flow cytometry controls for FIG. 19. The C-peptide/NKX6-1 control is the same as shown in FIG. 16. (C) Real-time PCR analysis of Stage 6 cells with or without resizing treated with Alk5i or DMSO (n=3). Data generated with the 1013-4FA cell line.

FIG. 14A-FIG. 14B shows resized and unresized Stage 6 clusters have SMAD⅔ phosphorylation and reduced GSIS with Alk5i treatment. (A) Western blot of Stage 6 cells with and without resizing stained for phosphorylated SMAD ⅔ (pSMAD⅔), total SMAD ⅔ (tSMAD⅔), and Actin. (B) Human insulin secretion of Stage 6 cells in static GSIS assay resized or unresized with treatment of DMSO or Alk5i. All data shown is from 1013-4FA.

FIG. 15A-FIG. 15I is a series of illustrations, images, and graphs depicting the state of the cytoskeleton controls expression of the transcription factors. NEUROG3 and NKX6-1 in pancreatic progenitors. (a) Schematic of the differentiation protocols used for suspension differentiation and plate down studies. (b) Images of clusters at the beginning of stage 4 dispersed and plated onto ECM-coated TCP for culture for the remainder of the protocol. Scale bar=100 μm. (c) qRT-PCR of pancreatic genes at the end of stage 4 of cells plated on collagen I at the beginning of stage 4 compared to regular suspension cluster or clusters reaggregated after dispersion (Tukey's HSD test, n=4). (d) qRT-PCR of pancreatic genes at the end of stage 4 of cells plated on varying heights of collagen 1 gels at the beginning of stage 4. Increasing the height of collagen I gels fixed to TCP correlates with decreasing the effective stiffness experienced by cells (ANOVA, n=4). (e) qRT-PCR of plated stage 4 cells treated with a screen of cytoskeletal modifying compounds to identify latrunculin A as potent endocrine inducer. XXi, a γ-secretase inhibitor, was used as a positive control (Dunnett's multiple comparisons test, n=4). (f) Immunostaining of plated cells at the end of stage 4 demonstrating that a 1 μM latrunculin A treatment increases NEUROG3+ and decreases NKX6-1+ cells. Scale bar=50 μm. (g) Latrunculin A dose response of pancreatic gene expression added during stage 4 measured with qRT-PCR (ANOVA, n=4). (h) Immunostaining of plated stage 4 cells treated for 24 hours with 1 μM latrunculin, demonstrating depolymerization of F-actin but maintenance of PDX1 expression. (i) Western blot quantification of the G/F actin ratio within cells under different culture formats and treated with latrunculin A (n=3). All data was generated with HUES8. All data error bars represent SEM. ns=not significant, *=p<0.05, **=p<0.01, ***=p<0.001.

FIG. 16A-FIG. 16C is a series of projections, plots, and graphs depicting single-cell RNA sequencing demonstrating that cytoskeletal state directs pancreatic progenitor fate. (a) tSNE projection of single-cell RNA sequencing performed on plated stage 4 cells and treated with either 0.5 μM latrunculin A or 5 μM nocodazole. Unsupervised clustering of the combined cell population from all three conditions revealed four separate clusters. (b) Violin plots indicating important upregulated genes in each cluster. (c) The percentage of cells within each cluster for each condition. All data was generated with HUES8.

FIG. 17A-FIG. 17I is a series of plots and images depicting Latrunculin A treatment during stage 5 drastically increased SC-β cell specification of plated pancreatic progenitors. (a) Flow cytometry two weeks into stage 6 for NKX6-1, CHGA, and C-peptide of plated cells as per FIG. 15(a), untreated or treated with 0.5 μM latrunculin A throughout stage 4, 5, or 6 (Dunnett's multiple comparisons test, n=4). (b) Static GSIS two weeks into stage 6 of plated cells, untreated or treated with 0.5 μM latrunculin A throughout stage 4, 5, or 6 (paired t-test compares between low and high glucose for a particular sample, Dunnett's test compares insulin secretion at high glucose to the control, n=4). (c) Optimization of latrunculin A concentration and timing during stage 5 for plated cells. Static GSIS was performed after 2 weeks of stage 6 (t-tests, n=4). (d) Insulin content of plated cells two weeks into stage 6, untreated or treated 24 hour with 1 μM latrunculin A (unpaired t-tests, n=4). (e) Proinsulin/insulin ratio of plated cells two weeks into stage 6, untreated or treated 24 hour with 1 μM latrunculin A (unpaired t-tests, n=4). (f) qRT-PCR measuring pancreatic (left) and non-pancreatic (right) gene expression of plated cells two weeks into stage 6, untreated or treated 24 hour with 1 μM latrunculin A (unpaired t-tests, n=4). (g) Immunostaining for AFP and C-peptide of plated cells two weeks into stage 6, untreated or treated 24 hour with 1 μM latrunculin A. Scale bar=100 μm. (h) Images of aggregation of plated cells after one week in stage 6. (i) Dynamic glucose-stimulated insulin secretion of stage 6 cells exhibiting first and second phase insulin release. All data was generated with HUES8. All data error bars represent SEM. ns=not significant, *=p<0.05, **=p<0.01, ***=p<0.001.

FIG. 18A-FIG. 18J is a series of illustrations, graphs, and images depicting SC-β cells differentiated with the new planar protocol expressing β cell markers and function in vitro. (a) Schematic of the new planar protocol for making SC-β cells incorporating a 1 μM latrunculin A treatment for the first 24 hour of stage 5. (b) Flow cytometry after one week in stage 6 of cells from HUES8 with and without stage 5 latrunculin A treatment measuring endocrine induction (CHGA+) and SC-β cell specification (C-peptide+/NKX6-1+) (unpaired t-tests, n=4). (c) Flow cytometry of islet and SC-β cells markers for stage 6 cells differentiated from HUES8, 1013-4FA, and 1016SeVA hPSC lines (n=4). (d) qRT-PCR of islet and disallowed genes for stage 6 cells and human islets (Dunnett's multiple comparisons test, n=4 for SC-β cells, n=3 for human islets). (e) Immunostaining of aggregated planar stage 6 cells from HUES8. (f) Insulin content of stage 6 cells (n=4). (g) Proinsulin/insulin content ratio for stage 6 cells (n=4). (h) Static GSIS for stage 6 cells (paired t-tests, n=4). (i) Dynamic GSIS for planar stage 6 cells generated from HUES8 (n=7), 1013-4FA (n=3), and 1016SeVA (n=4). Suspension stage 6 data is replotted from Velazco-Cruz et al.⁵ (HUES8, n=12; 1013-4FA, n=3; 1016SeVA, n=4). (j) Planar static GSIS data from (i) plotted together compared to human islet data replotted from Velazco-Cruz et al.⁵ (n=12). All data shown in this figure is of cells generated with the planar differentiation protocol unless otherwise noted. All data error bars represent SEM. ns=not significant, *=p<0.05, **=p<0.01, ***=p<0.001.

FIG. 19A-FIG. 19C is a series of graphs and images depicting SC-β cells generated with the new planar protocol can rapidly cure pre-existing diabetes in mice. (a) Diabetes was induced with STZ in a total of 19 mice. 4 weeks after injection, SC-β cells generated with the planar protocol were transplanted into 12 of these mice. 5 non-diabetic mice served as controls. Glucose tolerance tests were performed 3, 10, and 13 weeks after transplantation. A nephrectomy was performed 12 weeks after transplantation (Tukey's HSD test, ‡=different than no transplant, §=different than transplant, #=different than untreated control). (b) In vivo GSIS of mice receiving the SC-β cell transplant 2 and 10 weeks after transplantation measuring human insulin. ns=not significant, *=p<0.05, **=p<0.01, ***=p<0.001. (c) Immunostaining of sectioned kidneys transplanted with SC-β cells 3 weeks after transplantation showing C-peptide+ cells. All data was generated with HUES8 using the planar protocol outlined in FIG. 19A. All data error bars represent SEM.

FIG. 20A-FIG. 20G is a series of heat maps, plots, and images showing the state of the cytoskeleton influences endodermal cell fate. (a) Suspension and plated pancreatic progenitors differentiated to stage 6 as per FIG. 15(a) either untreated, treated with 0.5 μM latrunculin A throughout stage 4, or treated with 1 μM latrunculin A for the first 24 hours of stage 5. Bulk RNA sequencing at two weeks into stage 6 was used to generate a heat map of the 1000 most differentially expressed genes between the stage 5 latrunculin A treatment and plated control. (b) Heat map from bulk RNA sequencing of select genes from multiple endodermal lineages. (c) Volcano plot from bulk RNA sequencing data showing expression differences of select genes between untreated plated cells and stage 5 latrunculin treated cells. (d) Gene enrichment analysis from bulk RNA sequencing of select gene sets from multiple endodermal lineages. (e) Immunostaining (left) and qRT-PCR (right) of cells differentiated with an exocrine differentiation protocol treated with latrunculin A or nocodazole (Dunnett's multiple comparisons test, n=4). (f) Immunostaining (left) and qRT-PCR (right) of cells differentiated with an intestinal differentiation protocol treated with latrunculin A or nocodazole (Dunnett's multiple comparisons test, n=4). (g) Immunostaining (left) and qRT-PCR (right) of cells differentiated with a hepatic differentiation protocol treated with latrunculin A or nocodazole (Dunnett's multiple comparisons test, n=4). Scale bars=50 μm. All data was generated with HUES8. All data error bars represent SEM. ns=not significant, *=p<0.05, **=p<0.01, ***=p<0.001.

FIG. 21A-FIG. 21D is a series of images and bar graphs. (a) Images of pancreatic progenitors plated at beginning of stage 4 onto ECM-coated TCP as per FIG. 15(a). Scale bar=200 μm. (b) qRT-PCR of plated cells at the end of stage 4 (n=4). (c) A colorimetric antibody-based integrin adhesion assay at the beginning and end of stage 4 confirmed high expression of integrin subunits that bind to collagens I and IV (α1, α2, β1), fibronectin (αV, β1, α5β1), vitronectin (αV, β1, αVβ5) and some but not all laminin isoforms (α3, β1). Data is normalized to an isotype control. All data was generated with HUES8.

FIG. 22A-FIG. 22H is a series of plots and heat maps. (a) Latrunculin A dose response of pancreatic gene expression added during stage 4 from 1013-4FA and 1016SeVA measured with qRT-PCR (n=4). (b) qRT-PCR of pancreatic gene expression at the end of stage 4 in response to latrunculin B dosing on plated HUES8 (ANOVA, n=4). (c) qRT-PCR of untreated HUES8 plated stage 4 cells, untreated reaggregated clusters, and reaggregated clusters treated with the actin polymerizer jasplakinolide (unpaired t-tests, n=4). (d) tSNE plot heat map generated from single-cell RNA sequencing data of plated HUES8 pancreatic progenitors showing expression of pancreatic genes. All data generated as per FIG. 15(a). All data error bars represent SEM. ns=not significant, *=p<0.05, **=p<0.01, ***=p<0.001.

FIG. 23A-FIG. 23H (a) qRT-PCR of HUES8 cells differentiated with the new planar protocol to the end of stage 4, untreated or treated throughout stage 4 with 0.5 μM latrunculin A (unpaired t-tests, n=4). (b-d) qRT-PCR of HUES8 cells differentiated with the planar protocol to stage 6 with or without a 24 hour 1 μM latrunculin A treatment at the beginning of stage 5, (b,c) showing expression of islet and β cell genes and (d) non-pancreatic genes (unpaired t-tests, n=4). (e, f) Immunostaining of aggregates generated from the planar protocol with (e) 1013-4FA and (f) 1016SeVA iPSC lines. Scale bars=50 μm. (g) Quantification of mouse C-peptide with ELISA of serum from mice. (h) Quantification of human insulin in the serum of mice without a transplant. All data was generated with HUES8 with the new planar protocol was per FIG. 18(a). All data error bars represent SEM. ns=not significant, *=p<0.05, **=p<0.01, ***=p<0.001.

### DETAILED DESCRIPTION OF THE INVENTION

The present disclosure is based, at least in part, on the discovery that a modified process can produce cells that can respond to glucose appropriately to near islet-like levels, demonstrating both a first phase and second phase response. As described herein is a protocol to generate beta-like cells from human pluripotent stem cells with dynamic insulin secretion. Furthermore, the present disclosure is based, at least in part, on the discovery that modulation of the actin cytoskeleton can enhance pancreatic differentiation of human pluripotent stem cells.

Generating Beta-Like Cells from Human Pluripotent Stem Cells with Dynamic Insulin Secretion

It was discovered that the currently described method generated stem cell-derived beta (SC-6) cells function better (undergoing glucose-stimulated insulin secretion) than cells in the published literature (Pagliuca et al. Cell 2014) and express beta cell markers. This includes increased insulin secretion with a static assay and having first and second phase insulin response in a dynamic assay.

As described herein, stem cell-derived beta (SC-6) cells can be useful as a cellular therapy for diabetes or for drug screening. The presently disclosed process enhances differentiation of human pluripotent stem cells to insulin-producing beta cells. This process is modified from a previously described 6-step differentiation protocol published by Pagliuca et al. Cell 2014. With this new process, cells that can respond to glucose appropriately to near islet-like levels have been generated, demonstrating both a first phase and second phase response.

In order to achieve the above modulation, the following was performed: (1) shorten stage 3 to 1 day; (2) allow for TGFb signaling in stage 6 by removal of Alk5 inhibitor II (current literature includes this inhibitor); (3) remove T3 from stage 6 (current literature includes this inhibitor); (4) perform stage 6 in a serum-free basal media (formulation included); and (5) break apart and reaggregate clusters at the beginning of stage 6.

Using the above modulations, enhanced stem cell-derived beta cells that better perform glucose-stimulated insulin secretion were generated. The field currently includes Alk5 inhibitor II and T3 during the last stage of culture to mature stem cell-derived beta cells. The field has been unable to generate functional stem cell-derived beta cells that have both first phase and second phase insulin secretion (see Rezania et al. Nature Biotechnology 2014 for the poor dynamic function stem cell-derived beta cells have in the field).

For example, Example 1 describes methods for generating stem cell derived beta-like (SC-6) cells. It was discovered that a differentiation strategy focusing on modulating TGFβ signaling, controlling cellular cluster size, and using an enriched serum-free media (ESFM) to generate SC-β cells that express β cell markers and undergo GSIS with first and second phase dynamic insulin secretion.

Modulation of the Actin Cytoskeleton Enhances Pancreatic Differentiation of Human Pluripotent Stem Cells

As described herein, this work has identified the actin cytoskeleton as a crucial regulator of human pancreatic cell fate. By controlling the state of the cytoskeleton with either cell arrangement (two- vs three-dimensional), substrate stiffness, or directly with chemical treatment, it is shown herein that a polymerized cytoskeleton prevents premature induction of NEUROG3 expression in pancreatic progenitors, but also inhibits subsequent differentiation to SC-β cells.

As shown herein, it was discovered that modulation of the actin cytoskeleton and its downstream effector Yes-Associated Protein (YAP) at specific time points during differentiation can enhance differentiation of human pluripotent stem cells to cells of endodermal lineage, pancreatic progenitors, and insulin-producing beta cells. Using a 6-step differentiation protocol modified from Pagliuca et al. Cell 2014, the following specific features were observed: (1) actin polymerization and YAP activity during Stage 4 enhances generation of pancreatic progenitors (PDX1+/NKX6-1+/SOX9+); (2) actin depolymerization and loss of YAP activity during Stage 5, preferentially during the first 24-48 hr of Stage 5, enhances generation of endocrine cells, specifically beta cells that demonstrate enhanced glucose-stimulated insulin secretion.

In order to achieve the above modulation, the following can be performed: (1) promoting actin polymerization by plating onto stiff surfaces, such as tissue culture plastic with a thin layer of ECM protein to promote attachment; (2) promoting actin depolymerization by plating onto soft surfaces, such as hydrogels, or by treating cells with latrunculin A and/or latrunculin B; (3) promoting YAP transcriptional activity using the same methods to promote actin polymerization; and/or (4) inhibiting YAP transcriptional activity using the same methods to promote actin depolymerization or by treatment with Verteporfin.

Using the above modulations, enhanced stem cell-derived beta cells were generated to better perform glucose-stimulated insulin secretion than previous methods and can be generated on attachment culture. Currently in the field, stem cell-derived beta cells can be generated but do not function as well as with the presently disclosed approach. The field does not utilize actin cytoskeleton and YAP signaling in their protocols. The field is also unable to generate functional stem cell-derived beta cells with the cells in attachment culture—it must either be done in suspension aggregates (the control for many experiments in the attached data set, first reported in Pagliuca et al. Cell 2014) or in aggregates on an air-liquid-interface (first reported in Rezania et al. Nature Biotechnology 2014).

Described herein is the generation of stem cell-derived beta cells that function better (undergoing glucose-stimulated insulin secretion) than cells in the published literature (Pagliuca et al. Cell 2014) and express beta cell markers.

Also described herein are methods for the generation of stem cell-derived beta cells in a planar protocol that can undergo glucose-stimulated insulin secretion (GSIS).

Also described herein is the demonstration that cells can be detached from a plate, either using UpCell technology that does not require cell dispersion or by dispersing and reaggregating the cells, and maintain insulin secretion capacity, better enabling transplantation.

Also described herein is the generation of pancreatic progenitor cells that have reduced endocrine expression (such as expression of NGN3, NEUROD1) and increased pancreatic progenitor expression (such as expression of NKX6-1, SOX9).

Pancreatic progenitors and stem cell-derived beta cells can be useful as a cellular therapy for diabetes. Stem cell-derived beta cells are also useful for drug screening. The presently disclosed attachment culture approach yields a convenient platform for drug screening studies.

The presently disclosed culture approach can also facilitate enhanced quality and reproducibility of the differentiations and is conducive to automation of the differentiation process for commercialization.

An an example, differentiation protocols, as described in example 2, by cytoskeletal modulation can generate cells of several lineages (e.g., SC-13, beta-like cells). It was discovered that the state of the actin cytoskeleton is critical to endodermal cell fate choice. By utilizing a combination of cell-biomaterial interactions as well as small molecule regulators of the actin cytoskeleton (e.g., a cytoskeletal-modulating agent), the timing of endocrine transcription factor expression can be controlled to modulate differentiation fate and develop a two-dimensional protocol for differentiating cells. Importantly, this new planar protocol greatly enhances the function of SC-β cells differentiated from induced pluripotent stem cell (iPSC) lines and forgoes the requirement for three-dimensional cellular arrangements.

Different degrees of actin polymerization at specific points of differentiation biased cells toward different endodermal lineages, and thus non-optimal cytoskeletal states led to large inefficiencies in cell specification.

Furthermore, the methods described herein can control actin polymerization to direct differentiations of these other endodermal cell fates to modulate lineage specification.

Other lineages that can be generated according to the provided methods can be liver, esophageal, exocrine, pancreas, intestine, or stomach.

A cytoskeletal-modulating agent can be any agent that promotes or inhibits actin polymerization or microtubule polymerization. For example, the cytoskeletal-modulating agent can be an actin depolymerization or polymerization agent, a microtubule modulating agent, or an integrin modulating agent (e.g., compounds, such as antibodies and small molecules). For example, the cytoskeletal-modulating agent can be latrunculin A, latrunculin B, nocodazole, cytochalasin D, jasplakinolide, blebbistatin, y-27632, y-15, gdc-0994, or an integrin modulating agent. The cytoskeletal-modulating agent can be any cytoskeletal-modulating agent known in the art (see e.g., Ley et al. Nat Rev Drug Discov. 2016 March; 15(3): 173-183).

Cell Cluster Resizing

Resizing of cell clusters can be performed by any methods known in the art. For example, cell resizing can comprise breaking apart cell clusters and reaggregating. As another example, the cell clusters can be resized by incubating in a cell-dissociating reagent and passed through a cell strainer (e.g., a 100 μm nylon cell strainer). As another example, cells can be resized by single cell dispersing with TrypLE and reaggregating.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of cells as described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of using generated cells for cell replacement therapies or stem cell transplant. For example, the disclosed compositions and methods can be used to treat diabetes or other disease associated with dysfunctional endodermal cells in a subject in need administration of a therapeutically effective amount of cells of endodermal lineage or beta cells, so as to induce insulin secretion.

Methods described herein are generally performed on a subject in need thereof. A subject in need of the therapeutic methods described herein can be a subject having, diagnosed with, suspected of having, or at risk for developing a diabetes or other disease associated with dysfunctional endodermal cells. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of cells of endodermal lineage (e.g., hepatocytes, insulin-expressing cells (e.g., β cells, SC-β cells), intestinal cells) is, for example, that amount that would cause the desired therapeutic effect in a subject while minimizing undesired side effects.

In various embodiments, an effective amount of endodermal lineage or beta cells described herein can respond to glucose by secretion of insulin. In various embodiments, an effective amount of cells described herein can treat diabetes or other disease associated with dysfunctional endodermal cells, substantially inhibit diabetes or other disease associated with dysfunctional endodermal cells, slow the progress of diabetes or other disease associated with dysfunctional endodermal cells, or limit the development of diabetes or other disease associated with dysfunctional endodermal cells.

According to the methods described herein, administration can be a cell transplantation, cell implantation, parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of beta cells or cells of endodermal lineage can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to induce insulin secretion.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Shamel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of cells of endodermal lineage or beta cells can occur as a single event or over a time course of treatment. For example, cells of endodermal lineage or beta cells can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for diabetes or other disease associated with dysfunctional endodermal cells.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be implantation, transplantation, parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving direct injection (e.g., systemic or stereotactic), transplantation, or implantation of generated cells, oral ingestion, cell-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the cells in a manner similar to that used for delivering insulin or chemotherapy to specific organs or tumors. Typically, using such a system, cells can be administered in combination with a biodegradable, biocompatible polymeric implant that contains or releases the cells over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: improve the transport of the therapeutic cells to its site of action; allow colocalized deposition with other agents or excipients; improve the stability of the cells in vivo; prolong the residence time of the cells at the site of action by reducing clearance; decrease the nonspecific delivery of the cells to nontarget tissues; alter the immunogenicity of the agent; decrease dosage frequency; or improve shelf life of the product.

Screening

Also provided are methods for screening. The screening method can comprise providing a generated cell by any of the methods described herein and introducing a compound or composition (e.g., a secretagogue) to the cell. For example, the screening method can be used for drug screening or toxicity screening on any cell of endodermal lineage or beta cell provided herein.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: Chem Bridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to stem cells, media, and factors as described herein. Such packaging of the components separately can, if desired, be presented in a package, pack, or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Acquisition of Dynamic Function in Human Stem Cell-Derived Beta Cells

The following example describes a new six-stage differentiation strategy to improve functional maturation of stem cell-derived β (SC-β) cells, which secrete large amounts of insulin and are glucose-responsive, displaying both first and second phase insulin release. Also described herein is the dynamic function in stem cell-derived β cells.

Recent advances in human pluripotent stem cell (hPSC) differentiation protocols have generated insulin-producing cells resembling pancreatic β cells. While these stem cell-derived β (SC-β) cells are capable of undergoing glucose-stimulated insulin secretion (GSIS), insulin secretion per cell remains low compared to islets and lack clear first and second phase dynamic insulin release. Herein, this work reports a differentiation strategy focused on modulating TGFβ signaling, controlling cellular cluster size, and using an enriched serum-free media (ESFM) to generate SC-β cells that express β cell markers and undergo GSIS with first and second phase dynamic insulin secretion. Transplantation of these cells into mice greatly improves glucose tolerance. These results reveal that specific time frames (or periods of time) for inhibiting and permitting TGFβ signaling are required during SC-β cell differentiation to achieve dynamic function. The capacity of these cells to undergo GSIS with dynamic insulin release makes them a promising cell source for diabetes cellular therapy.

### Introduction

Diabetes mellitus is a global health problem affecting over 400 million people worldwide and is increasing in prevalence. Diabetes is principally caused by the death or dysfunction of insulin-producing β cells found within islets of Langerhans in the pancreas, resulting in improper insulin secretion and failure of patients to maintain normal glycemia, which in severe cases can cause ketoacidosis and death. Patients are often reliant on insulin injections but can still suffer from long-term complications, including retinopathy, neuropathy, nephropathy, and cardiovascular disease. An alternative treatment is replacement of the endogenous β cells by transplantation of pancreatic islets. While this therapy has had clinical success, limited availability of cadaveric donor islets largely hampers its widespread application.

Differentiation of hPSCs into stem cell-derived β cells (SC-β cells) is a promising alternative cell source for diabetes cell replacement therapy as well as other applications, such as modeling disease and studying pancreatic development. Through modulation of pathways identified from embryonic development, studies with hPSCs have detailed protocols for generating cells that resemble early endoderm and pancreatic progenitors, the latter of which can be transplanted into rodents and spontaneously differentiated into β-like cells after several months.

Approaches for generating SC-β cells in vitro have been published that in part use the compound Alk5 inhibitor type II (Alk5i) to inhibit TGFβ signaling during the last stages of differentiation³⁰. These approaches produced SC-β cells for the first time capable of undergoing GSIS in static incubations, express β cell markers, and control blood sugar in diabetic mice after several weeks. However, these cells had inferior function compared to human islets, including lower insulin secretion and little to no first and second phase insulin release in response to a high glucose challenge, demonstrating that these SC-β cells were less mature than β cells from islets. Several follow-up studies have been performed introducing new differentiation factors or optimizing the process but have failed to bring SC-β cell function equivalent to human islets^{14,26,36,55}.

Here this work demonstrates a new six-stage differentiation strategy that generates almost pure populations of endocrine-containing β-like cells that secrete high levels of insulin and express β cell markers by modulating Alk5i exposure to inhibit and permit TGFβ signaling during key stages in combination with cellular cluster resizing and ESFM culture. These cells are glucose-responsive, exhibiting first and second phase insulin release, and respond to multiple secretagogues. Transplanted cells greatly improve glucose tolerance in mice. This work demonstrates that inhibiting TGFβ signaling during Stage 6 greatly reduces the function of these differentiated cells while treatment with Alk5i during Stage 5 is necessary for a robust β-like cell phenotype.

Results

Differentiation to Glucose-Responsive SC-β Cells In Vitro

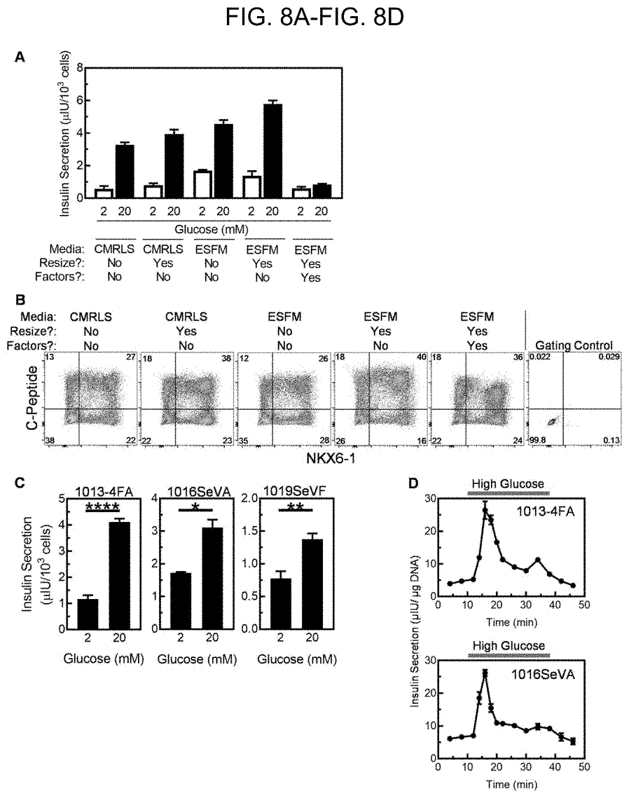

An improved differentiation protocol was developed using the HUES8 cell line. Y27632 was included during Stages 3-4 and activin A during Stage 4 to help maintain cluster integrity and shortened Stage 3 from 2 to only 1 day to enhance progenitors. An ESFM was also developed for Stage 6 to replace the serum-containing media used previously to have a serum-free protocol. During protocol pilot studies, both resizing clusters and removal Alk5i and T3 was observed to increase insulin secretion while maintaining the C-peptide+ population (see e.g., FIG. 8A-FIG. 8B).

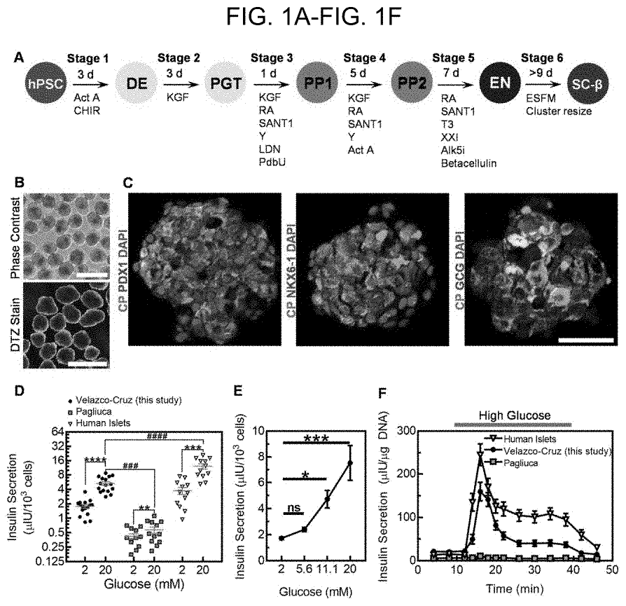

Combining these modifications resulted in the new six-stage differentiation protocol outlined in FIG. 1A. Stage 6 cells are grown as clusters in suspension culture (see e.g., FIG. 1B) that averaged 172±34 μm (mean±standard deviation; n=353 individual clusters) in diameter, less than half the diameter of the clusters before resizing, which was 364±55 μm (n=155 individual clusters). Stage 6 clusters stained red for the zinc-chelating dye dithizone (DTZ), which stains β cells. Immunostaining of sectioned clusters revealed most cells to be C-peptide+, a protein also produced by the INS gene, in addition to PDX1+ and NKX6-1+, β cell markers (see e.g., FIG. 1C). A subset of cells stained positive for glucagon (GCG+) or were polyhormonal, staining positive for both C-peptide and GCG. These polyhormonal cells are known to not to resemble adult β cells and are not functional.

Function was tested for Stage 6 cells generated with the new differentiation protocol using both static (see e.g., FIG. 1D-FIG. 1E; FIG. 8C) and dynamic GSIS assays (see e.g., FIG. 1F, FIG. 8D) and found that not only do the cells secrete insulin but also increase insulin release when moved from low to high glucose. With static GSIS, while there was some variability, Stage 6 cells increased insulin secretion on average by a factor of 3.0±0.1 when moved from 2 to 20 mM glucose, an improvement compared to cells generated from a previously published protocol (1.4±0.1), referred to here as the Pagliuca protocol³⁰, but less than human islets (3.2±0.1) on average (see e.g., FIG. 1D). Stage 6 cells from this study did not increase insulin secretion in response to 5.6 mM glucose but did increase secretion in response to higher concentrations (11.1 and 20 mM), indicating that the cells are not stimulated by a low glucose threshold (see e.g., FIG. 1E). In terms of insulin secretion per cell, Stage 6 cells secreted on average 5.3±0.5 μlU10³ cells at 20 mM glucose, 9.2±1.1 times more than cells generated with the Pagliuca protocol and 2.3±0.3 times less than human islets, on average (see e.g., FIG. 1D).

With dynamic GSIS, Stage 6 cells displayed a rapid first phase insulin release within 3-5 min of high glucose exposure, increasing insulin secretion by a factor of 7.6±1.3 to 159±21 μlUμg DNA, higher than Stage 6 cells generated from the Pagliuca protocol (1.7±0.2× increase to 11±1 μlUμg DNA) but lower than human islets (15.0±2.4× increase to 245±26 μlUμg DNA) (see e.g., FIG. 1F). Second phase insulin secretion was observed with continued high glucose exposure, with cells maintaining 2.1±0.3 higher insulin secretion than the initial low glucose, a higher increase than with the Pagliuca protocol (0.9±0.1) but lower than human islets (6.7±0.8) (see e.g., FIG. 1F). When the cells were returned to low glucose, insulin secretion from Stage 6 cells returned to a reduced rate. Elevating insulin secretion and displaying first and second phase insulin release to a high glucose challenge are key features of β cell behavior. Overall, Stage 6 cells generated with this differentiation strategy produced cells with clear first and second phase insulin secretion, which was not demonstrated by Pagliuca³⁰ and not seen with Stage 6 cells produced with the Pagliuca protocol. However, when compared to human islets containing β cells, these Stage 6 cells still have lower insulin secretion per cell at high glucose, lower glucose stimulation on average, and slightly slower first phase insulin release.

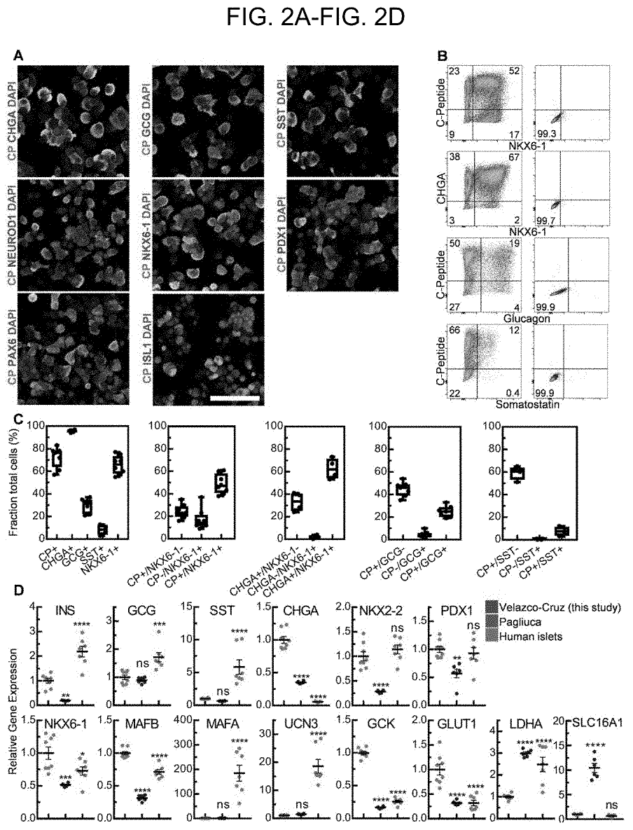

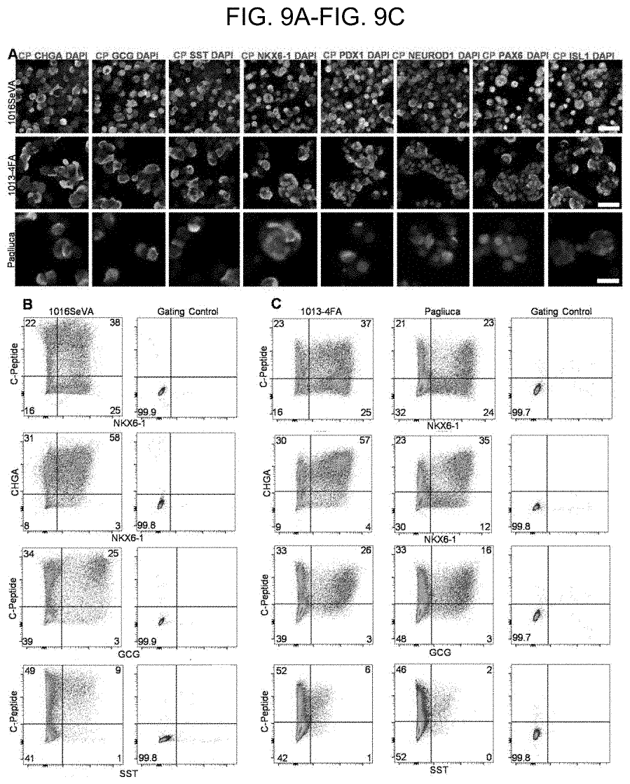

To further characterize Stage 6 cells generated with the new differentiation protocol, cells were immunostained with a panel of pancreatic islet markers (see e.g., FIG. 2A-2C, FIG. 9). The vast majority of cells expressed chromogranin A (96±1%), a pan-endocrine marker, and most cells expressed C-peptide (73±3%) (see e.g., FIG. 2). These fractions are higher than in Stage 6 cells generated with the Pagliuca protocol (see e.g., FIG. 9) and those previously reported³⁰. Many C-peptide+ cells from both protocols expressed other markers found in β cells and expression of the other pancreatic hormones was observed (see e.g., FIG. 2, FIG. 9). The majority of C-peptide+ cells expressed NKX6-1 (see e.g., FIG. 2) and were monohormonal, which was presumed to be the SC-13 cell population. The fraction of C-peptide+ cells not expressing another hormone was increased compared to Stage 6 cells generated with the Pagliuca protocol and that previously reported³⁰ while the fraction of these cells expressing another hormone was comparable (see e.g., FIG. 2, FIG. 9). This data shows that Stage 6 cells generated with this new strategy are predominantly pancreatic endocrine with the majority expressing C-peptide.

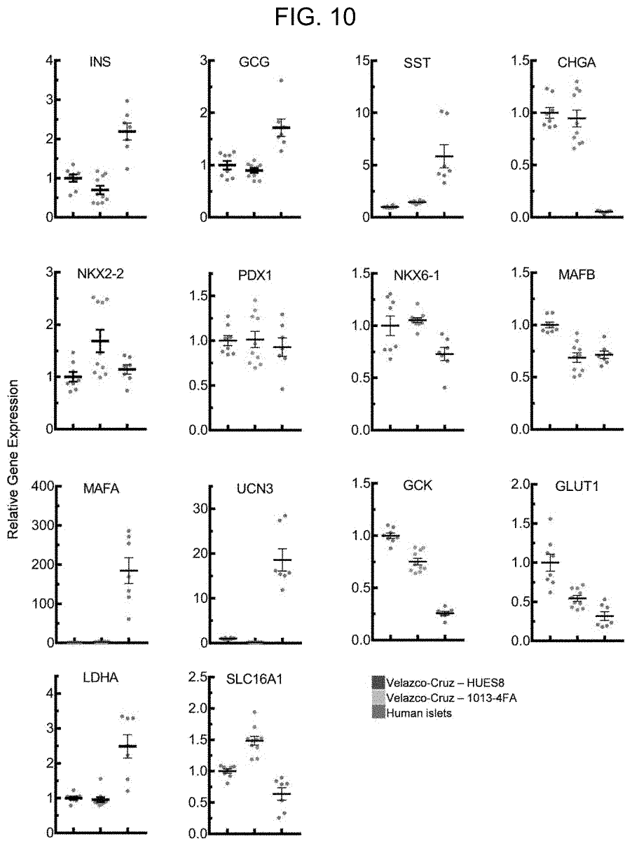

Expression of several genes was measured comparing Stage 6 cells generated with the Pagliuca protocol, Stage 6 cells generated with the protocol from this work, and human islets (see e.g., FIG. 2D and FIG. 10). Many islet and β cell genes were increased compared to the Pagliuca protocol, including INS, CHGA, NKX2-2, PDX1, NKX6-1, MAFB, GCK and GLUT1. Interesting, LDHA and SLC16A1, disallowed β cell genes, had reduced expression in the Stage 6 cells compared to both the Pagliuca protocol and human islets (LDHA) and the Pagliuca protocol (SLC16A1). The Stage 6 cells generated from the protocol in this work had increased expression of CHGA, NKX6-1, MAFB, GCK, and GLUT1 compared to human islets. However, INS, GCG, SST, and particularly MAFA and UCN3 had reduced expression compared to Stage 6 cells. However, several recent reports have provided evidence that question the utility of MAFA and UCN3 in evaluating human SC-β cell maturation. MAFA expression is low in juvenile human β cells. MAFB is expressed in human but not mouse β cells. UCN3 expression is much higher in mouse than human β cells and is also expressed by human a cells. This data shows that the Stage 6 cells generated in this work have improved gene expression for many markers compared to the Pagliuca protocol and, while the expression of several β cell markers are equal to or great than human islets, other markers remain low.

Transplantation of SC-β Cells into Glucose-Intolerant Mice

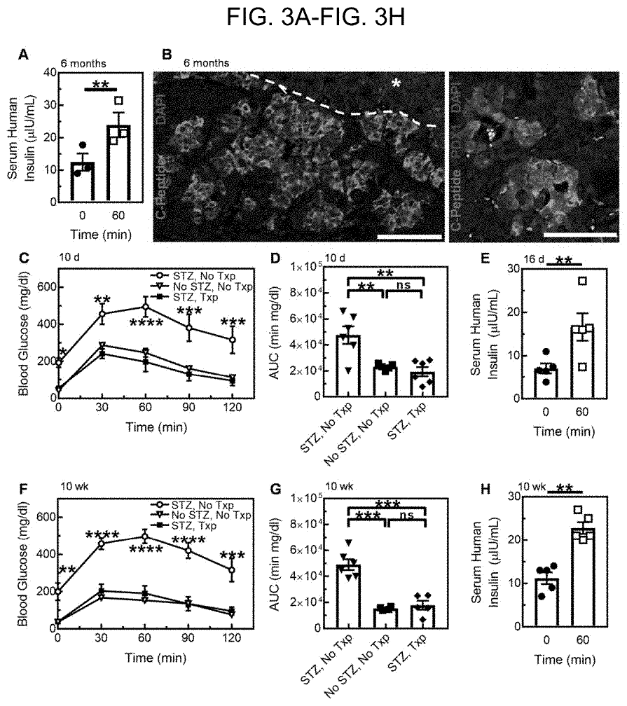

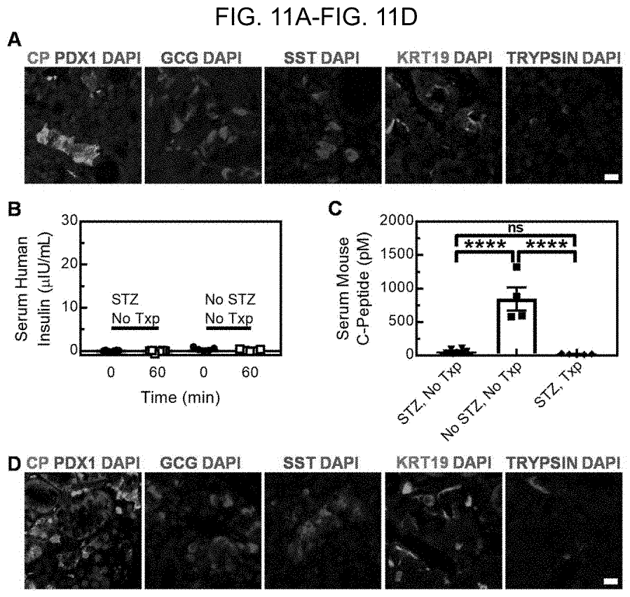

To evaluate the functional potential of Stage 6 cells in vivo, cells were first transplanted under the renal capsule of non-diabetic mice and the ability of the graft to respond to a glucose challenge was evaluated (see e.g., FIG. 3A). Even after extended time post-transplantation (6 months), the grafts responded to a glucose injection by increasing human insulin by a factor of 1.9±0.5. Excision and immunostaining of the transplanted kidneys revealed C-peptide+ cells that tended to be clustered together in addition to other pancreatic endocrine and exocrine markers (see e.g., FIG. 3B; FIG. 11A). To more rigorously evaluate Stage 6 cells in vivo, a separate mouse cohort that had been chemically induced to be diabetic with streptozotocin (STZ) was transplanted and function was evaluated at early (10 and 16 d) and late (10 wk) time points. After only 10 d post-transplantation, STZ-treated mice receiving Stage 6 cells had greatly improved glucose tolerance compared to STZ-treated sham mice and had similar glucose clearance as the no STZ-treated mice (see e.g., FIG. 3C-FIG. 3D). Measurements of human insulin 16 d after transplantation revealed high insulin concentration that increased by a factor of 2.3±0.6 with a glucose injection to 16.6±3.1 μlU/mL (see e.g., FIG. 3E). These values are greater than what was reported previously³⁰ under similar conditions, which had an insulin increase of 1.4±0.3 and concentration of 3.8±0.8 μlU/mL. Observing the cohort 10 wk after transplantation revealed similar results as the 10 d and 16 d data, with transplanted mice having greatly improved glucose tolerance (see e.g., FIG. 3F-FIG. 3G) and glucose-responsive insulin secretion (see e.g., FIG. 3H). Mice not receiving STZ had similar glucose tolerance as mice receiving a therapeutic dose of human islets. Mice that did not receive Stage 6 cells had undetectable human insulin and mice that received STZ had drastically reduced mouse C-peptide compared to non-STZ treated mice (see e.g., FIG. 11B-FIG. 11C). Grafts from these STZ-treated mice contained cells that expressed β cell markers in addition to other endocrine and exocrine markers (see e.g., FIG. 11D). Overall this data demonstrates that Stage 6 cells generated with the new protocol are functional both at early and late time points in vivo, greatly improving glucose tolerance to equal that of non-STZ-treated mice.

Characterization of SC-β Cell Dynamic Function

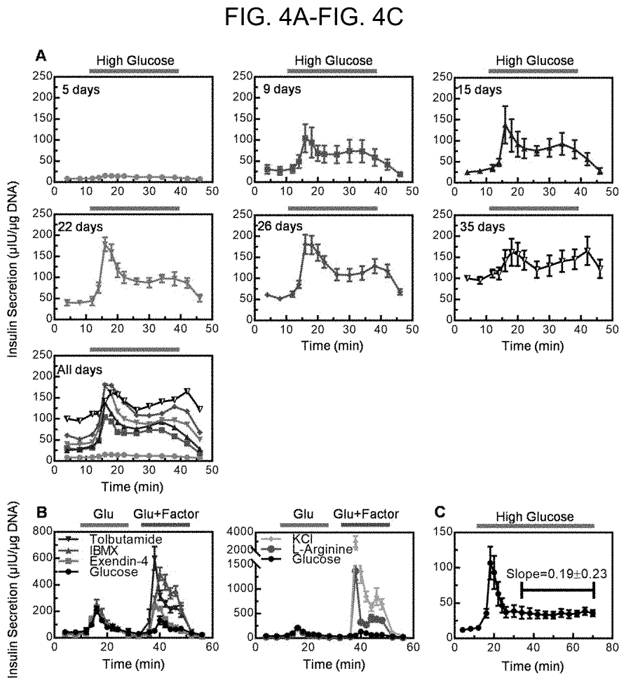

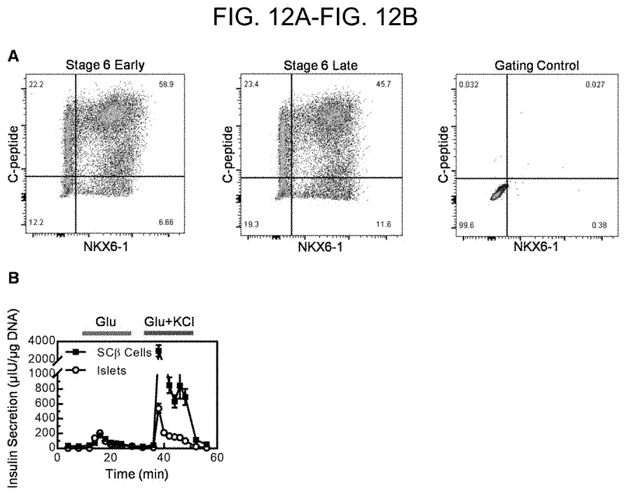

Since the differentiation protocol produces cells that are capable of dynamic insulin secretion, this phenotype was studied in more detail. A dynamic GSIS was performed on cells as they progressed through Stage 6 (see e.g., FIG. 4A). Robust dynamic function was transient, with cells at 5 d secreting low amounts of insulin and exhibiting weak first and second phases while later time points (9-26 d) secreting higher amounts of insulin with a clear first and second phase response. During this time, the fraction of C-peptide+ cells decreased slightly (see e.g., FIG. 12A). By 35 d, insulin secretion at low glucose had risen such that first and second phase were difficult to clearly identify. This data shows that SC-β cells require 9 d in Stage 6 to acquire dynamic function, this function persists for weeks, but after extended in vitro culture glucose-responsiveness is lost. Similarly, cadaveric human islets are known to have a limited functional lifetime in vitro, and the cause of this is not clear. This data further suggests an optimal time frame for these cells to be used in transplantation and drug screening studies. To further characterize dynamic insulin secretion, perifusion experiments were performed to assay whether SC-β cells could respond to sequential challenges with several known secretagogues (see e.g., FIG. 4B). After an initial high glucose challenge, SC-β cells were able to respond to a second high glucose-only challenge, albeit less strongly than the first challenge, and extending the first glucose challenge to 1 hr in a separate experiment did not reduce insulin secretion (see e.g., FIG. 4C). Addition of other secretagogues during the second challenge further increased insulin secretion (see e.g., FIG. 4B). Membrane depolarizers KCl and L-Arginine had the largest increases. Tolbutamide (blocks potassium channel), 3-isobutyl-1-methylxanthine (IBMX; raises cytosolic cAMP), and exendin-4 (agonist of GLP-1 receptor) also increased insulin secretion over high glucose alone. Not only was insulin secretion increased but it rose faster than with high glucose alone. However, the response of Stage 6 cells to KCl challenge was stronger than in human islets (see e.g., FIG. 12B), an observation made by others comparing β-like cells to human islets, possibly indicative of continued immature or juvenile β cell phenotype. Taken together, these data show that SC-β cells can respond to several secretagogues that have diverse modes of action and have potential application in drug screening.

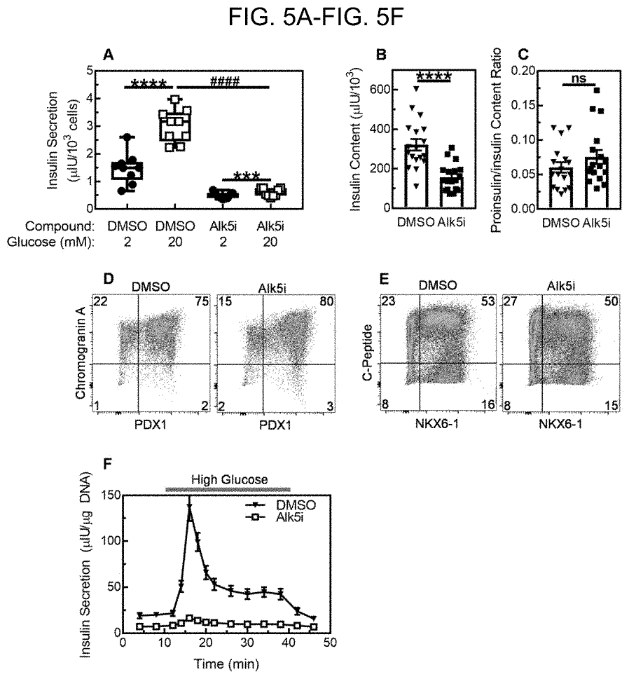

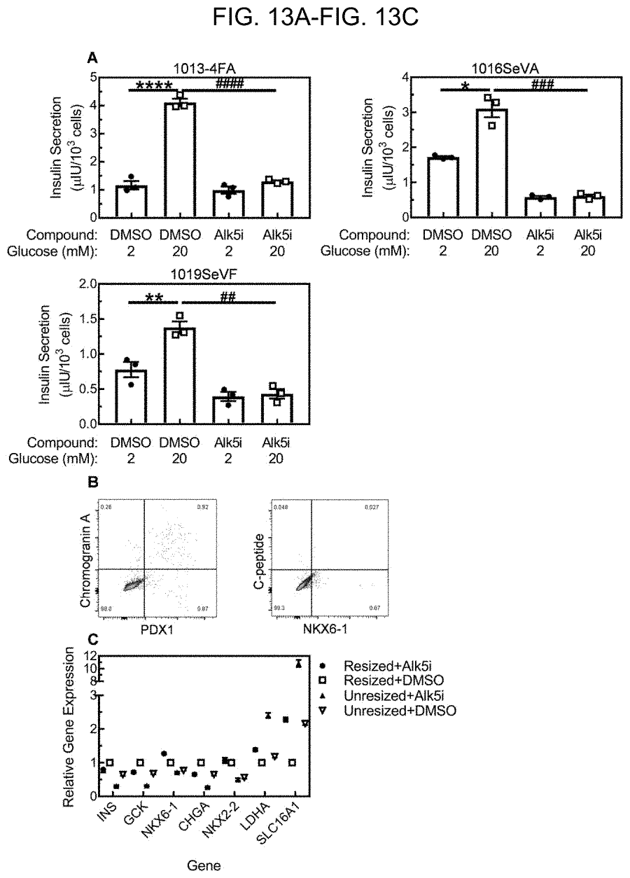

Role of TGFβ Signaling in SCβ Cell Differentiation and Maturation After having evaluated SC-β cells generated with the new protocol, the protocol changes that were made were investigated in order to gain insights into SC-β cell differentiation and maturation. While inclusion of Alk5i during Stage 6 resulted in relatively weak but statistically significant GSIS in a static assay, similar to data from the Pagliuca protocol (see e.g., FIG. 1D), omission of Alk5i drastically increased insulin secretion and glucose stimulation (see e.g., FIG. 5A and FIG. 13A). Insulin content also increased with removal of Alk5i during Stage 6 (see e.g., FIG. 5B), but the proinsulin/insulin ratio remained similar (see e.g., FIG. 5C), suggesting the increased insulin content is not due to hormone processing. Furthermore, the fraction of cells expressing pancreatic endocrine markers, including C-peptide, remained similar between DMSO- and Alk5i-treated cells (see e.g., FIG. 5D-FIG. 5E, FIG. 13B). Gene expression was similar overall with and without Alk5i treatment, with cluster resizing typically having a larger effect (see e.g., FIG. 13C). Cells treated with Alk5i during Stage 6 also had dramatically reduced insulin secretion with the dynamic GSIS assay, displaying weak to no first and second phase response (see e.g., FIG. 5F) similar to cells generated with the Pagliuca protocol (see e.g., FIG. 1F). This data shows that Alk5i treatment during Stage 6 inhibits functional maturation of SC-β cells.

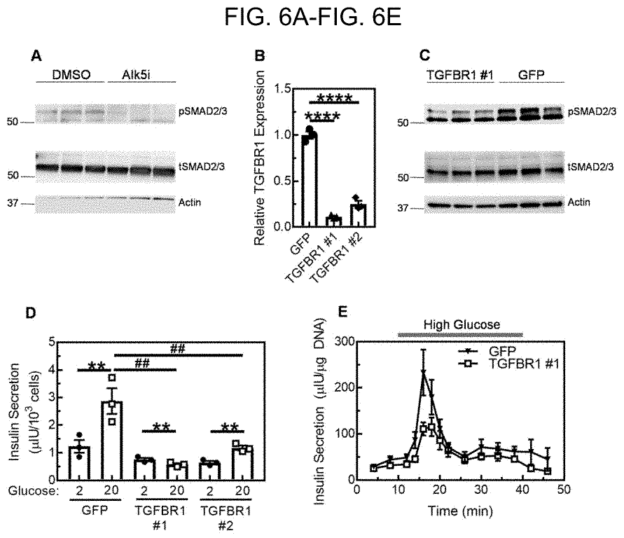

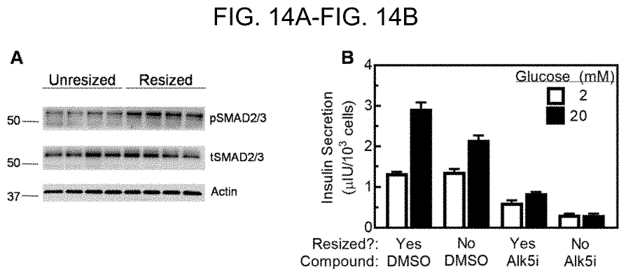

The studies with Alk5i during Stage 6 suggested that permitting TGFβ signaling was necessary for robust functional maturation of SC-β cells, as inhibition of TGFBR1 is the canonical function of Alk5i. To test this hypothesis, western blot analysis was used to validate that TGFβ signaling was occurring in the Stage 6 cells via SMAD phosphorylation (see e.g., FIG. 6A). Alk5i treatment diminished phosphorylated SMAD, confirming that TGFβ signaling was indeed occurring and inhibited by Alk5i. SMAD phosphorylation was observed in Stage 6 clusters regardless of whether they were resized, consistent with observations that Alk5i treatment reduced GSIS regardless of resizing (see e.g., FIG. 14). Next, two lentiviruses were generated carrying shRNA designed to knockdown TGFBR1 (TGFBR1 #1 and #2). These viruses were capable of reducing TGFBR1 transcript compared to control virus targeting GFP in Stage 6 cells (see e.g., FIG. 6B) and reduced SMAD phosphorylation (see e.g., FIG. 6C, FIG. 14), albeit to much lesser extent than Alk5i treatment (see e.g., FIG. 6A). Similar to Alk5i treatment (see e.g., FIG. 5A, FIG. 5F), Stage 6 cells transduced with shRNA against TGFBR1 had reduced insulin secretion and reduced positive glucose responsiveness in the static GSIS assay (see e.g., FIG. 6C) and blunted glucose-response in the dynamic GSIS assay (see e.g., FIG. 6D). This data shows permitting TGFβ signaling during Stage 6 is important for SC-β cell functional maturation, which is inhibited by treatment with Alk5i.

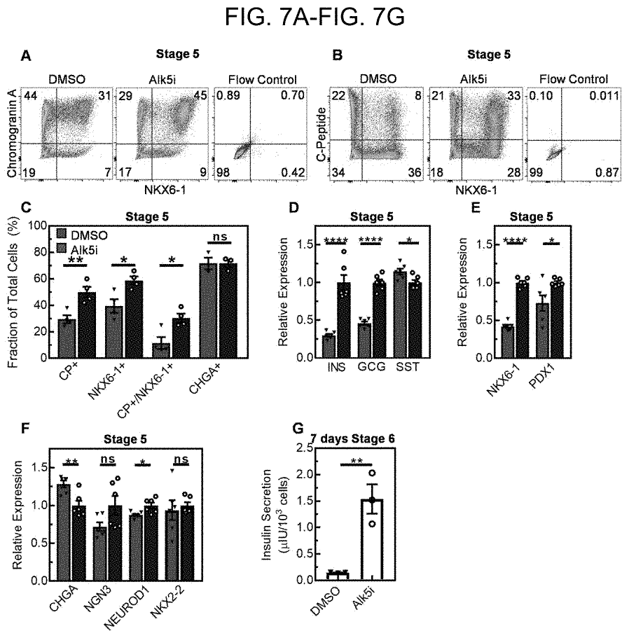

Finally, the role of Alk5i was studied during Stage 5 of differentiation to evaluate its effects on differentiation toward pancreatic endocrine cells. These experiments were performed as outlined in FIG. 1A in the presence or absence of Alk5i. The fraction of cells differentiated to endocrine cells (CHGA+) was unchanged but the fraction of cells differentiated to a C-peptide+ phenotype was decreased by omitting Alk5i (see e.g., FIG. 7A-FIG. 7C). Similarly, the fraction of cells co-expressing C-peptide and NKX6-1, an important transcription factor for specifying β cells, was decreased by omitting Alk5i. INS and GCG gene expression decreased with Alk5i omission, but surprisingly SST expression was slightly increased (see e.g., FIG. 7D). Expression of NKX6-1 and PDX1 were reduced without Alk5i (see e.g., FIG. 7E) while expression of several pancreatic endocrine markers were either unchanged or only slightly changed (see e.g., FIG. 7F). To further test the importance of Alk5i during Stage 5, cells treated with or without Alk5i during Stage 5 were further cultured for 7 d in Stage 6 without Alk5i nor cluster resizing, and insulin secretion was substantially higher in cells treated with Alk5i during Stage 5 (see e.g., FIG. 7G). Taken together, these data show that Alk5i treatment during Stage 5 positively influences specification to β-like cell fate, not necessary to specify endocrine cells, and is necessary for high insulin secretion of resulting SC-β cells. In addition, these observations illustrate the importance of stage-specific treatment of the TGFβ signaling-inhibitor Alk5i to both generate and functionally mature SC-β cells.

### Discussion

This work demonstrates that enhanced functional maturation of SC-β cells is achieved with a new six-stage differentiation strategy. These cells secrete a large amount of insulin and are glucose-responsive, displaying both first and second phase insulin release. This differentiation procedure generates almost pure endocrine cell populations without selection or sorting, and most cells express C-peptide and other β cell markers. Upon transplantation into STZ-treated mice, glucose tolerance is rapidly restored and function persists for months. These SC-β cells respond to multiple secretagogues in a perifusion assay. Modulating TGFβ signaling was crucial for success, with inhibition during Stage 5 increasing SC-β cell differentiation but inhibition during Stage 6 reducing function and insulin content. Permitting TGFβ signaling during Stage 6 was necessary for robust dynamic function.

SC-β cells generated by previously reported protocols^{39,32} do not produce robust first and second phase insulin release in response to glucose stimulation. Both protocols inhibited TGFβ signaling during the final stage of differentiation, and many subsequent reports also include inhibitors of TGFβ signaling without demonstrating proper dynamic function. However, a major observation of the current study is that correct modulation of TGFβ signaling during key cell transition and maturation steps is critical for successful differentiation to functional SC-β cells, with permitting TGFβ signaling being required for improved functional maturation during Stage 6.

SC-β cells in this report were able to control glucose in STZ-treated mice rapidly within 10 d. Currently, a key limitation in diabetes cell replacement therapy is the need for sustainable source of functional β cells and improving the quality of SC-β cells to be transplanted helps overcome this challenge. The process of making SC-β cells demonstrated by this work is scalable, with the cells grown and differentiated as clusters in suspension culture. The use of cellular clusters in suspension culture allows flexibility for many applications, such as large animal transplantation studies or therapy (order 10⁹ cells).

This strategy enhances the utility of in vitro-differentiated SC-β cells for drug screening due to their improved kinetics. Proper dynamic insulin release is an important feature of β cell metabolism that is commonly lost in diabetes. This work has established a renewable resource of SC-β cells with dynamic insulin release that can be used to better study the mechanism of β cell failure in diabetes and demonstrated their response to several secretagogues.

The culmination of numerous modifications to the protocol produced SC-β cells exhibiting dynamic glucose response. In addition to modulating TGFβ signaling, other notable changes included the removal of serum, reducing cluster size, and the lack of several additional factors (T3, N-acetyl cysteine, Trolox, and R428) used in other reports during the last stage. While this work demonstrated reproducibility of the protocol across multiple cell lines, marker expression and function were greatest in the HUES8 cell line.

Methods

Culture of Undifferentiated Cells

Undifferentiated hPSC lines were cultured using mTeSR1 in 30-mL spinner flasks on a rotator stir plate spinning at 60 RPM in a humidified 5% CO₂ 37° C. incubator. Cells were passaged every 3-4 d by single cell dispersion. The HUES8 hESC line, 1013-4FA (a non-diabetic hiPSC line), 1016SeVA (a non-diabetic hiPSC line), and 1019SeVF (a type 1-diabetic hiPSC line) have been previously published^{26,30}. Undifferentiated cells were cultured using mTeSR1 (StemCell Technologies; 05850) in 30-mL spinner flasks (REPROCELL; ABBWVS03A) on a rotator stir plate (Chemglass) spinning at 60 RPM in a humidified 5% CO₂ 37° C. incubator. Cells were passaged every 3-4 days by single cell dispersion using Accutase (StemCell Technologies; 07920), viable cells counted with Vi-Cell XR (Beckman Coulter) and seeded at 6×10⁵ cells/mL in mTeSR1+10 μM Y27632 (Abcam; ab120129).

Cell Line Differentiation

To initiate differentiation, undifferentiated cells were single-cell dispersed using Accutase and seeded at 6×10⁵ cells/mL in mTeSR1+10 μM Y27632 in a 30-ml spinner flask. Cells were then cultured for 72 hr in mTeSR1 and then cultured in the following differentiation media. Stage 1 (3 days): S1 media+100 ng/ml Activin A (R&D Systems; 338-AC)+3 μM Chir99021 (Stemgent; 04-0004-10) for 1 day. S1 media+100 ng/ml Activin A for 2 days. Stage 2 (3 days): S2 media+50 ng/ml KGF (Peprotech; AF-100-19). Stage 3 (1 day): S3 media+50 ng/ml KGF+200 nM LDN193189 (Reprocell; 040074)+500 nM PdBU (MilliporeSigma; 524390)+2 μM Retinoic Acid (MilliporeSigma; R2625)+0.25 μM Sant1 (MilliporeSigma; S4572)+10 μM Y27632. Stage 4 (5 days): S4 media+5 ng/mL Activin A+50 ng/mL KGF+0.1 μM Retinoic Acid+0.25 μM SANT1+10 μM Y27632. Stage 5 (7 days): S5 media+10 μM ALK5i II (Enzo Life Sciences; ALX-270-445-M005)+20 ng/mL Betacellulin (R&D Systems; 261-CE-050)+0.1 μM Retinoic Acid+0.25 μM SANT1+1 μM T3 (Biosciences; 64245)+1 μM XXI (MilliporeSigma; 595790). Stage 6 (7-35 days): ESFM.

Differentiation media formulations used were the following. S1 media: 500 mL MCDB 131 (Cellgro; 15-100-CV) supplemented with 0.22 g glucose (MilliporeSigma; G7528), 1.23 g sodium bicarbonate (MilliporeSigma; S3817), 10 g bovine serum albumin (BSA) (Proliant; 68700), 10 μL ITS-X (Invitrogen; 51500056), 5 mL GlutaMAX (Invitrogen; 35050079), 22 mg vitamin C (MilliporeSigma; A4544), and 5 mL penicillin/streptomycin (P/S) solution (Cellgro; 30-002-CI). S2 media: 500 mL MCDB 131 supplemented with 0.22 g glucose, 0.615 g sodium bicarbonate, 10 g BSA, 10 μL ITS-X, 5 mL GlutaMAX, 22 mg vitamin C, and 5 mL P/S. S3 media: 500 mL MCDB 131 supplemented with 0.22 g glucose, 0.615 g sodium bicarbonate, 10 g BSA, 2.5 mL ITS-X, 5 mL GlutaMAX, 22 mg vitamin C, and 5 mL P/S. S5 media: 500 mL MCDB 131 supplemented with 1.8 g glucose, 0.877 g sodium bicarbonate, 10 g BSA, 2.5 mL ITS-X, 5 mL GlutaMAX, 22 mg vitamin C, 5 mL P/S, and 5 mg heparin (MilliporeSigma; A4544). ESFM: 500 mL MCDB 131 supplemented with 0.23 g glucose, 10.5 g BSA, 5.2 mL GlutaMAX, 5.2 mL P/S, 5 mg heparin, 5.2 mL MEM nonessential amino acids (Corning; 20-025-CI), 84 μg ZnSO₄ (MilliporeSigma; 10883), 523 μL Trace Elements A (Corning; 25-021-CI), and 523 μL Trace Elements B (Corning; 25-022-CI). Cells were sometimes cultured with 0.01% DMSO. Cells were resized the first day of Stage 6 by incubating in Gentle Cell Dissociation Reagent (StemCell Technologies; 07174) for 8 min, washed with ESFM, passed through a 100 μm nylon cell strainer (Corning; 431752), and cultured in ESFM in 6-well plates on an Orbi-Shaker (Benchmark) set at 100 RPM. Assessment assays were performed between 10-16 days of stage 6 unless otherwise stated. Human islets were acquired from Prodo Labs for comparison. A subset of Stage 6 experiments were performed without cluster resizing, with Alk5i and T3, with Alk5i, and/or CMRL 1066 Supplemented (CMRLS) (Mediatech; 99-603-CV)+10% fetal bovine serum (FBS) (HyClone; 16777)+1% P/S rather than ESFM, as indicated. To perform the Pagliuca protocol, the protocol outlined in Pagliuca, Millman, alter et al. 2014³⁰ was followed in 30-mL spinner flasks.

Light Microscopy

Light Microscopy images were taken of unstained or stained with 2.5 μg/mL DTZ (MilliporeSigma; 194832) cell clusters using an inverted light microscope (Leica DMi1).

Immunostaining

To immunostain in vitro cell clusters or ex vivo transplanted grafts within mouse kidneys, samples were fixed with 4% paraformaldehyde (Electron Microscopy Science; 15714) overnight at 4° C. After fixation, cell clusters were embedded in Histogel (Thermo Scientific; hg-4000-012). Embedded cell clusters and grafts were placed in 70% ethanol and submitted for paraffin-embedding and sectioning. Paraffin was removed using Histoclear (Thermo Scientific; C78-2-G), samples rehydrated, and antigens retrieved with 0.05 M EDTA (Ambion; AM9261) in a pressure cooker (Proteogenix; 2100 Retriever). Samples were blocked and permeabilized for 30-min with staining buffer (5% donkey serum (Jackson Immunoresearch, 017-000-121) and 0.1% Triton-X 100 (Acros Organics; 327371000) in PBS), stained overnight with primary antibodies at 4° C., stained for 2 hr with secondary antibodies at 4° C., and treated with mounting solution DAPI Fluoromount-G (SouthernBiotech; 0100-20). To immunostain plated cells, clusters were single-cell dispersed using TrypIE Express (Fisher, 12604039), plated down onto Matrigel (Fisher, 356230)-coated plates, cultured in ESFM for 16 hr, and fixed for 30 min with 4% paraformaldehyde at RT. Fixed cells were blocked and permeabilized with staining buffer for 45 min at RT, stained overnight with primary antibodies at 4° C., stained for 2 hr with secondary antibodies at RT, and stained with DAPI for 5 min. Imaging was performed on a Nikon A1Rsi confocal microscope or Leica DM14000 fluorescence microscope.

Primary antibody solutions were made in staining buffer with the following antibodies at 1:300 dilution unless otherwise noted: rat-anti-C-peptide (DSHB, GN-ID4-S), 1:100 mouse-anti-nkx6.1 (DSHB, F55A12-S), mouse-anti-glucagon (ABCAM, ab82270), goat-anti-pdx1 (R&D Systems; AF2419), rabbit-anti-somatostatin (ABCAM, ab64053), mouse-anti-pax6 (BDBiosciences; 561462), rabbit-anti-chromogranin a (abl 5160), goat-anti-neurodl (R&D Systems; AF2746), mouse-anti-Islet1 (DSHB, 40.2d6-s), 1:100 mouse-anti-cytokeratin 19 (Dako; M0888), undiluted rabbit-anti-glucagon (Cell Marque; 259A-18), 1:100 sheep-anti-trypsin (R&D Systems; AF3586). Secondary antibody solutions were made in staining buffer with the following antibodies at 1:300 dilution: anti-rat-alexa fluor 488 (Invitrogen; a21208), anti-mouse-alexa fluor 594 (Invitrogen; a21203), anti-rabbit-alexa fluor 594 (Invitrogen; a21207), anti-goat-alexa fluor 594 (Invitrogen; a11058).

Static GSIS

Assays were performed by collecting ˜20-30 stage 6 clusters or cadaveric human islets, washed twice with KRB buffer (128 mM NaCl, 5 mM KCl, 2.7 mM CaCl₂) 1.2 mM MgSO₄, 1 mM Na₂HPO₄, 1.2 mM KH₂PO₄, 5 mM NaHCO₃, 10 mM HEPES (Gibco; 15630-080), and 0.1% BSA), resuspended in 2 mM glucose KRB, and placed into transwells (Corning; 431752) in 24-well plates. Clusters were incubated at 2 mM glucose KRB for a 1 hr equilibration. The transwell was then drained and transferred into a new 2 mM glucose KRB well, discarding the old KRB solution. Clusters were again incubated for 1 hr at low glucose and then the transwell is drained and transferred into a new 2, 5.6, 11.1, or 20 mM glucose KRB well, retaining the old 2 mM glucose KRB. Clusters were then incubated for 1 hr at high glucose and then the transwell was drained and the old glucose KRB was retained. The retained KRB was run with the Human Insulin Elisa (ALPCO; 80-INSHU-E10.1) to quantify insulin secretion. The cells were single-cell dispersed by TrypLE treatment, counted on a Vi-Cell XR, and viable cell counts used to normalize insulin secretion.

Dynamic Glucose-Stimulated Insulin Secretion

A perifusion system was assembled, as has been previously reported⁵. The system used a high precision 8-channel dispenser pump (ISMATEC; ISM931C) in conjunction with 0.015″ inlet and outlet two-stop tubing (ISMATEC; 070602-04i-ND) connected to 275-μl cell chamber (BioRep; Pen-Chamber) and dispensing nozzle (BioRep; PERI-NOZZLE) using 0.04″ connection tubbing (BioRep; Peri-TUB-040). Solutions, tubing, and cells were maintained at 37° C. in a water bath. Stage 6 clusters and cadaveric human islets were washed with KRB twice and resuspended in 2 mM glucose KRB. Cells were then loaded onto a Biorep perifusion chamber sandwiched between two layers of Bio-Gel P-4 polyacrylamide beads (Bio-Rad; 150-4124). Cells were perfused with 2 mM glucose KRB for 90 min prior to sample collection for equilibration. For single high glucose challenges, sample collection was started with cells exposed to 2 mM glucose KRB for 12 min, followed by 24 min of 20 mM glucose KRB, and back to 2 mM glucose KRB for an additional 12 min. For multiple secretagogue challenges, sample collection was started with cells exposed to 2 mM glucose KRB for 6 min, followed by 12 min of 20 mM glucose KRB, 6 min 2 mM glucose KRB, 12 min of 20 mM glucose KRB plus treatment, and finally 6 min of 2 mM glucose KRB. Treatments with multiple secretagogues were as follows: 20 mM glucose only, 10 nM Extendin-4 (MilliporeSigma; E7144), 100 μM IBMX (MilliporeSigma; 15879), 300 μM Tolbutamide (MilliporeSigma; T0891), 20 mM L-Arginine (MilliporeSigma; A5006), and 30 mM KCL (Thermo Fisher; BP366500). Effluent was collected at a 100 μl/min flow rate with 2-4 min collection points. After sample collection, clusters were collected and lysed in 10 mM Tris (MilliporeSigma; T6066), 1 mM EDTA, and 0.2% Triton-X 100 solution and DNA was quantified using Quant-iT Picogreen dsDNA assay kit (Invitrogen; P7589). Insulin secretion was quantified using the Human Insulin Elisa kit.

Flow Cytometry

Clusters were single-cell dispersed with TrypLE, fixed with 4% paraformaldehyde for 30 min at 4° C., blocked and permeabilized with staining buffer for 30 min at 4° C., incubated with primary antibodies in staining buffer overnight at 4° C., incubated with secondary antibodies in staining buffer for 2 hr at 4° C., resuspended in staining buffer, and analyzed on an LSRII (BD Biosciences) or X-20 (BD Biosciences). Dot plots and percentages were generated using FlowJo. All antibodies were used at 1:300 dilution except where noted. The antibodies used were: rat-anti-C-peptide, mouse-anti-nkx6.1 (1:100), mouse-anti-glucagon, rabbit-anti-somatostatin, rabbit-anti-chromogranin A (1:1000), goat-anti-pdx1, anti-rat-alexa fluor 488, anti-mouse-alexa fluor 647 (Invitrogen; a31571), anti-rabbit-alexa fluor 647 (Invitrogen; a31573), anti-goat-alexa fluor 647 (Invitrogen; a21447), anti-rabbit-alexa fluor 488 (Invitrogen; a21206).

Real-Time PCR

RNA was extracted using the RNeasy Mini Kit (Qiagen; 74016) with DNase treatment (Qiagen; 79254), and cDNA was synthesized using High Capacity cDNA Reverse Transcriptase Kit (Applied Biosystems; 4368814). Real-time PCR reactions were performed in PowerUp SYBR Green Master Mix (Applied Biosystems; A25741) on a StepOnePlus (Applied Biosystems) and analyzed using ΔΔCt methodology. TBP was used as a normalization gene.

TABLE 1
Primer sequences used
(gene, forward primer, reverse primer).

| | SEQ | | SEQ | |
|---|---|---|---|---|
| Gene | ID | Forward primer | ID | Reverse primer |
| name | NO. | sequence | NO. | sequence |
| INS | 1 | CAATGCCACGCTTC | 2 | TTCTACACACCCAAGACC |
| | | TGC | | CG |
| PDX1 | 3 | CGTCCGCTTGTTCT | 4 | CCTTTCCCATGGATGAAG |
| | | CCTC | | TC |
| GCC | 5 | AGCTGCCTTGTACC | 6 | TGCTCTCTCTTCACCTGC |
| | | AGCATT | | TCT |
| SST | 7 | TGGGTTCAGACAGC | 8 | CCCAGACTCCGTCAGTTT |
| | | AGCTC | | CT |
| TBP | 9 | GCCATAAGGCATCA | 10 | AACAACAGCCTGCCACCT |
| | | TTGGAC | | TA |
| NKX6-1 | 11 | CCGAGTCCTGCTTC | 12 | ATTCGTTGGGGATGACAG |
| | | TTCTTG | | AG |
| CHGA | 13 | TGACCTCAACGATG | 14 | CTGTCCTGGCTCTTCTGC |
| | | CATTTC | | TC |
| NEUROD1 | 15 | ATGCCCGGAACTTT | 16 | CATAGAGAACGTGGCAGC |
| | | TTCTTT | | AA |
| NGN3 | 17 | CTTCGTCTTCCGAG | 18 | CTATTCTTTTGCGCCGGT |
| | | GCTCT | | AG |
| NKX2-2 | 19 | GGAGCTTGAGTCCT | 20 | TCTACGACAGCAGCGACA |
| | | GAGGG | | AC |
| TGFBR1 | 21 | CGACGGCGTTACAG | 22 | CCCATCTGTCACACAAGT |
| | | TGTTTCT | | AAA |
| GUSB | 23 | CGTCCCACCTAGAA | 24 | TTGCTCACAAAGGTCACA |
| | | TCTGCT | | GG |
| UCN3 | 25 | GGAGGGAAGTCCAC | 26 | TGTAGAACTTGTGGGGGA |
| | | TCTCG | | GG |
| MAFA | 27 | GAGAGCGAGAAGTG | 28 | TTCTCCTTGTACAGGTCC |
| | | CCAACT | | CG |
| GCK | 29 | ATGCTGGACGACAG | 30 | CCTTCTTCAGGTCCTCCT |
| | | AGCC | | CC |
| MAFB | 31 | CATAGAGAACGTGG | 32 | ATGCCCGGAACTTTTTCT |
| | | CAGCAA | | TT |
| LDHA | 33 | GGCCTGTGCCATCA | 34 | GGAGATCCATCATCTCTC |
| | | GTATCT | | CC |
| GLUT1 | 35 | ATGGAGCCCAGCAG | 36 | GGCATTGATGACTCCAGT |
| | | CAA | | GTT |
| SLC16A1 | 37 | CACTTAAAATGCCA | 38 | AGAGAAGCCGATGGAAAT |
| | | CCAGCA | | GA |

Transplantation Studies

All animal work was performed in accordance to Washington University International Animal Care and Use Committee regulations. Mice were randomly assigned to transplantation or no transplantation groups, mouse number was chosen to be sufficient to allow for statistical significance based on prior studies. All procedures were performed by unblinded individuals. Two mouse cohorts were used in this study. The first consisted of non-STZ treated SCID/Beige male mice 50-56 days of age purchased from Charles River. The second consisted of STZ-treated and control-treated NOD/SCID male mice 6 weeks of age purchased from Jackson Laboratories. Mice were anaesthetized with isoflurane and injected with ˜5×10⁶ Stage 6 cells or saline (no transplant control) under the kidney capsule, similar to as previously reported. Mice were monitored up to 6 months after transplantation by performing glucose-tolerance tests and in vivo GSIS. Mice were fasted 16 hr and then injected with 2 g/kg of glucose. Blood was collected via tail bleed. Blood glucose levels were measured with a handheld glucometer (Contour Blood Glucose Monitoring System Model 9545C; Bayer). Human insulin was determined by collecting blood and separating serum in microvettes (Sarstedt; 16.443.100) and quantifying using the Human Ultrasensitive Insulin ELISA (ALPCO Diagnostics; 80-ENSHUU-E01.1). Serum mouse C-peptide concentration was determined by collecting blood from fed mice, separating serum in microvettes, and quantifying using a Mouse C-peptide ELISA (ALPCO Diagnostics; 80-CPTMS-E01).

Insulin and Proinsulin Content

Stage 6 clusters were washed thoroughly with PBS, immersed in a solution of 1.5% HCl and 70% ethanol, kept at −20° C. for 24 hr, retrieved and vortexed vigorously, returned and kept at −20° C. for an additional 24 hr, retrieved and vortexed vigorously, and centrifuged at 2100 RCF for 15 min. The supernatant was collected and neutralized with an equal volume of 1 M TRIS (pH 7.5). Human insulin and pro-insulin content were quantified using Human Insulin Elisa and Proinsulin Elisa (Mercodia; 10-1118-01) respectively. Samples were normalized to viable cell counts made using the Vi-Cell XR.

Western Blot

Protein was extracted from cell clusters after washing with PBS by placing in western blot lysis buffer consisting 50 mM HEPES, 140 mM NaCl (MilliporeSigma; 7647-14-5), 1 mM EDTA (MilliporeSigma, 1233508), 1% Triton X-100, 0.1% Na-deoxycholate (MilliporeSigma: D6750), 0.1% SDS (ThermoScientific; 24730020), 1 mM Na₃VO₄ (MilliporeSigma; 450243), 10 mM NaF (MilliporeSigma; S7920), and 1% Protease Inhibitor Cocktail (MilliporeSigma; p8340), incubating on a shaker for 15 min at 4° C., and centrifuging at 10000 RCF for 10 min at 4° C. Protein amount was quantified with the Pierce BCA Protein Assay (Thermo Scientific; 23228). Protein (30 μg) was loaded onto a 4-20% gradient polyacrylamide gel (Invitrogen; SP04200BOX), resolved by electrophoresis, and transferred onto a 0.45 μm nitrocellulose membrane (BioRad; 1620115). The nitrocellulose membrane was blocked with Blotting Grade Blocker (BioRad; 170-6404) and incubated with rabbit-anti-phospho-SMAD⅔ 1:1000 (Cell Signaling Technologies; 8828) and rabbit-anti-Actin 1:1000 (Santa Cruz Biotechnology; SC1616) antibodies in blocker overnight at 4° C. Membrane was washed and stained with rabbit secondary antibody 1:2500 (Jackson Immuno Research Laboratories; 211-032-171) in blocker for 2 hr at 4° C. and developed using SuperSignal West Femto (Thermo Scientific; 34096). Images were taken on an Odyssey FC (Li-COR). After imaging, the nitrocellulose membrane was stripped using Restore Western Blot Stripping Buffer (Thermo Scientific; 21059), incubated with rabbit-anti-SMAD⅔ (Cell Signaling Technologies; 8685) antibody overnight at 4° C., washed and stained with rabbit secondary antibody 1:2500 in blocker for 2 hr 4° C., developed using SuperSignal West Femto, and imaged using the Odyssey FC.

Lentivirus

pLKO.1 TRC plasmids containing shRNA sequences contained the following sequences: shRNA GFP, GCGCGATCACATGGTCCTGCT (SEQ ID NO: 89); shRNA TGFBR1 #1, GATCATGATTACTGTCGATAA (SEQ ID NO: 90); shRNA TGFBR1 #2, GCAGGATTCTTTAGGCTTTAT (SEQ ID NO: 91). Lentivirus particles were generated and titered using pMD-Lgp/RRE and pCMV-G, and RSV-REV packaging plasmids to contain shRNA. Stage 6 Day 1 cells were single cell dispersed using TrypLE, and 3 million cells were seeded in 4 mL ESFM lentivirus particles at MOI 3-5 on the shaker. Transduced cells were washed with fresh ESFM 16 hr post transduction. RNA extraction and static GSIS was performed on stage 6 day 13.

Statistical Analysis

Statistical significance was calculated using GraphPad Prism using the indicated statistical test. Slope and error in slope was calculated with the LINEST function in Excel. Data shown as mean±SEM unless otherwise noted or box-and-whiskers showing minimum to maximum point range, as indicated. n indicates the total number of independent experiments.

### REFERENCES

1. Amin, S., Cook, B., Zhou, T., Ghazizadeh, Z., Lis, R., Zhang, T., Khalaj, M., Crespo, M., Perera, M., Xiang, J. Z., et al. (2018). Discovery of a drug candidate for GLIS3-associated diabetes. Nat Commun 9, 2681.
2. Arda, H. E., Li, L., Tsai, J., Torre, E. A., Rosli, Y., Peiris, H., Spitale, R. C., Dai, C., Gu, X., Qu, K., et al. (2016). Age-Dependent Pancreatic Gene Regulation Reveals Mechanisms Governing Human beta Cell Function. Cell metabolism 23, 909-920.
3. Baron, M., Veres, A., Wolock, S. L., Faust, A. L., Gaujoux, R., Vetere, A., Ryu, J. H., Wagner, B. K., Shen-Orr, S. S., Klein, A. M., et al. (2016). A Single-Cell Transcriptomic Map of the Human and Mouse Pancreas Reveals Inter- and Intra-cell Population Structure. Cell Syst.
4. Bellin, M. D., Barton, F. B., Heitman, A., Harmon, J. V., Kandaswamy, R., Balamurugan, A. N., Sutherland, D. E., Alejandro, R., and Hering, B. J. (2012). Potent induction immunotherapy promotes long-term insulin independence after islet transplantation in type 1 diabetes. Am J Transplant 12, 1576-1583.
5. Bentsi-Barnes, K., Doyle, M. E., Abad, D., Kandeel, F., and Al-Abdullah, I. (2011). Detailed protocol for evaluation of dynamic perifusion of human islets to assess beta-cell function. Islets 3, 284-290.
6. Blum, B., Roose, A. N., Barrandon, O., Maehr, R., Arvanites, A. C., Davidow, L. S., Davis, J. C., Peterson, Q. P., Rubin, L. L., and Melton, D. A. (2014). Reversal of beta cell de-differentiation by a small molecule inhibitor of the TGFbeta pathway. eLife 3, e02809. Bonner-Weir, S., and Weir, G. C. (2005). New sources of pancreatic beta-cells. Nature biotechnology 23, 857-861.
8. Bruin, J. E., Asadi, A., Fox, J. K., Erener, S., Rezania, A., and Kieffer, T. J. (2015). Accelerated Maturation of Human Stem Cell-Derived Pancreatic Progenitor Cells into Insulin-Secreting Cells in Immunodeficient Rats Relative to Mice. Stem cell reports 5, 1081-1096.
9. Caumo, A., and Luzi, L. (2004). First-phase insulin secretion: does it exist in real life? Considerations on shape and function. Am J Physiol Endocrinol Metab 287, E371-385.
10. D'Amour, K., Bang, A., Eliazer, S., Kelly, O., Agulnick, A., Smart, N., Moorman, M., Kroon, E., Carpenter, M., and Baetge, E. (2006). Production of pancreatic hormone-expressing endocrine cells from human embryonic stem cells. Nature biotechnology 24, 1392-1793.
11. D'Amour, K. A., Agulnick, A. D., Eliazer, S., Kelly, O. G., Kroon, E., and Baetge, E. E. (2005). Efficient differentiation of human embryonic stem cells to definitive endoderm. Nature biotechnology 23, 1534-1541.
12. Del Prato, S., and Tiengo, A. (2001). The importance of first-phase insulin secretion: implications for the therapy of type 2 diabetes mellitus. Diabetes/metabolism research and reviews 17, 164-174.
13. Dhawan, S., Dirice, E., Kulkarni, R. N., and Bhushan, A. (2016). Inhibition of TGF-beta Signaling Promotes Human Pancreatic beta-Cell Replication. Diabetes 65, 1208-1218.
14. Ghazizadeh, Z., Kao, D. I., Amin, S., Cook, B., Rao, S., Zhou, T., Zhang, T., Xiang, Z., Kenyon, R., Kaymakcalan, O., et al. (2017). ROCKII inhibition promotes the maturation of human pancreatic beta-like cells. Nat Commun 8, 298.
15. Hering, B. J., Clarke, W. R., Bridges, N. D., Eggerman, T. L., Alejandro, R., Bellin, M. D., Chaloner, K., Czarniecki, C. W., Goldstein, J. S., Hunsicker, L. G., et al. (2016). Phase 3 Trial of Transplantation of Human Islets in Type 1 Diabetes Complicated by Severe Hypoglycemia. Diabetes Care 39, 1230-1240.
16. Hrvatin, S., O'Donnell, C. W., Deng, F., Millman, J. R., Pagliuca, F. W., Dilorio, P., Rezania, A., Gifford, D. K., and Melton, D. A. (2014). Differentiated human stem cells resemble fetal, not adult, beta cells. Proceedings of the National Academy of Sciences of the United States of America 111, 3038-3043.
17. Kayton, N. S., Poffenberger, G., Henske, J., Dai, C., Thompson, C., Aramandla, R., Shostak, A., Nicholson, W., Brissova, M., Bush, W. S., et al. (2015). Human islet preparations distributed for research exhibit a variety of insulin-secretory profiles. Am J Physiol Endocrinol Metab 308, E592-602.
18. Kroon, E., Martinson, L., Kadoya, K., Bang, A., Kelly, O., Eliazer, S., Young, H., Richardson, M., Smart, N., Cunningham, J., et al. (2008). Pancreatic endoderm derived from human embryonic stem cells generates glucose-responsive insulin-secreting cells in vivo. Nature biotechnology 26, 443-495.
19. Kudva, Y. C., Ohmine, S., Greder, L. V., Dutton, J. R., Armstrong, A., De Lamo, J. G., Khan, Y. K., Thatava, T., Hasegawa, M., Fusaki, N., et al. (2012). Transgene-Free Disease-Specific Induced Pluripotent Stem Cells from Patients with Type 1 and Type 2 Diabetes. Stem Cell Transl Med 1, 451-461.
20. Lacy, P. E., and Kostianovsky, M. (1967). Method for the isolation of intact islets of Langerhans from the rat pancreas. Diabetes 16, 35-39.
21. Lin, H. M., Lee, J. H., Yadav, H., Kamaraju, A. K., Liu, E., Zhigang, D., Vieira, A., Kim, S. J., Collins, H., Matschinsky, F., et al. (2009). Transforming growth factor-beta/Smad3 signaling regulates insulin gene transcription and pancreatic islet beta-cell function. The Journal of biological chemistry 284, 12246-12257.
22. Lyon, J., Manning Fox, J. E., Spigelman, A. F., Kim, R., Smith, N., O'Gorman, D., Kin, T., Shapiro, A. M., Rajotte, R. V., and MacDonald, P. E. (2016). Research-Focused Isolation of Human Islets From Donors With and Without Diabetes at the Alberta Diabetes Institute IsletCore. Endocrinology 157, 560-569.
23. Maehr, R., Chen, S., Snitow, M., Ludwig, T., Yagasaki, L., Goland, R., Leibel, R. L., and Melton, D. A. (2009). Generation of pluripotent stem cells from patients with type 1 diabetes. Proceedings of the National Academy of Sciences of the United States of America 106, 15768-15773.
24. Mathers, C. D., and Loncar, D. (2006). Projections of global mortality and burden of disease from 2002 to 2030. PLoS Med 3, e442.
25. McCall, M., and Shapiro, A. M. (2012). Update on islet transplantation. Cold Spring Harb Perspect Med 2, a007823.
26. Millman, J. R., and Pagliuca, F. W. (2017). Autologous Pluripotent Stem Cell-Derived beta-Like Cells for Diabetes Cellular Therapy. Diabetes 66, 1111-1120.
27. Millman, J. R., Xie, C., Van Dervort, A., Gurtler, M., Pagliuca, F. W., and Melton, D. A. (2016). Generation of stem cell-derived beta-cells from patients with type 1 diabetes. Nat Commun 7, 11463.
28. Nathan, D. M. (1993). Long-term complications of diabetes mellitus. N Engl J Med 328, 1676-1685.
29. Nostro, M. C., Sarangi, F., Yang, C., Holland, A., Elefanty, A. G., Stanley, E. G., Greiner, D. L., and Keller, G. (2015). Efficient Generation of NKX6-1(+) Pancreatic Progenitors from Multiple Human Pluripotent Stem Cell Lines. Stem cell reports 4, 591-604.
30. Pagliuca, F. W., Millman, J. R., Gurtler, M., Segel, M., Van Dervort, A., Ryu, J. H., Peterson, Q. P., Greiner, D., and Melton, D. A. (2014). Generation of functional human pancreatic beta cells in vitro. Cell 159, 428-439.
31. Petersen, M. B. K., Azad, A., Ingvorsen, C., Hess, K., Hansson, M., Grapin-Botton, A., and Honore, C. (2017). Single-Cell Gene Expression Analysis of a Human ESC Model of Pancreatic Endocrine Development Reveals Different Paths to beta-Cell Differentiation. Stem cell reports 9, 1246-1261.
32. Rezania, A., Bruin, J. E., Arora, P., Rubin, A., Batushansky, I., Asadi, A., O'Dwyer, S., Quiskamp, N., Mojibian, M., Albrecht, T., et al. (2014). Reversal of diabetes with insulin-producing cells derived in vitro from human pluripotent stem cells. Nature biotechnology 32, 1121-1133.
33. Rezania, A., Bruin, J. E., Riedel, M. J., Mojibian, M., Asadi, A., Xu, J., Gauvin, R., Narayan, K., Karanu, F., O'Neil, J. J., et al. (2012). Maturation of human embryonic stem cell-derived pancreatic progenitors into functional islets capable of treating pre-existing diabetes in mice. Diabetes 61, 2016-2029.
34. Rezania, A., Bruin, J. E., Xu, J., Narayan, K., Fox, J. K., O'Neil, J. J., and Kieffer, T. J. (2013). Enrichment of human embryonic stem cell-derived NKX6.1-expressing pancreatic progenitor cells accelerates the maturation of insulin-secreting cells in vivo. Stem Cells 31, 2432-2442.
35. Rieck, S., Bankaitis, E. D., and Wright, C. V. (2012). Lineage determinants in early endocrine development. Seminars in cell & developmental biology 23, 673-684.
36. Russ, H. A., Parent, A. V., Ringler, J. J., Hennings, T. G., Nair, G. G., Shveygert, M., Guo, T., Puri, S., Haataja, L., Cirulli, V., et al. (2015). Controlled induction of human pancreatic progenitors produces functional beta-like cells in vitro. The EMBO journal 34, 1759-1772.
37. Scharp, D. W., Lacy, P. E., Santiago, J. V., McCullough, C. S., Weide, L. G., Falqui, L., Marchetti, P., Gingerich, R. L., Jaffe, A. S., Cryer, P. E., et al. (1990). Insulin independence after islet transplantation into type I diabetic patient. Diabetes 39, 515-518.
38. Seino, S., Shibasaki, T., and Minami, K. (2011). Dynamics of insulin secretion and the clinical implications for obesity and diabetes. The Journal of clinical investigation 121, 2118-2125.
39. Shang, L., Hua, H., Foo, K., Martinez, H., Watanabe, K., Zimmer, M., Kahler, D. J., Freeby, M., Chung, W., LeDuc, C., et al. (2014). beta-cell dysfunction due to increased ER stress in a stem cell model of Wolfram syndrome. Diabetes 63, 923-933.
40. Shapiro, A. M., Lakey, J. R., Ryan, E. A., Korbutt, G. S., Toth, E., Warnock, G. L., Kneteman, N. M., and Rajotte, R. V. (2000). Islet transplantation in seven patients with type 1 diabetes mellitus using a glucocorticoid-free immunosuppressive regimen. N Engl J Med 343, 230-238.
41. Shapiro, A. M., Ricordi, C., Hering, B. J., Auchincloss, H., Lindblad, R., Robertson, R. P., Secchi, A., Brendel, M. D., Berney, T., Brennan, D. C., et al. (2006). International trial of the Edmonton protocol for islet transplantation. N Engl J Med 355, 1318-1330.
42. Simsek, S., Zhou, T., Robinson, C. L., Tsai, S. Y., Crespo, M., Amin, S., Lin, X., Hon, J., Evans, T., and Chen, S. (2016). Modeling Cystic Fibrosis Using Pluripotent Stem Cell-Derived Human Pancreatic Ductal Epithelial Cells. Stem Cells Transl Med 5, 572-579.
43. Song, J., and Millman, J. R. (2016). Economic 3D-printing approach for transplantation of human stem cell-derived beta-like cells. Biofabrication 9, 015002.
44. Stokes, A., and Preston, S. H. (2017). Deaths Attributable to Diabetes in the United States: Comparison of Data Sources and Estimation Approaches. PLoS One 12, e0170219.
45. Sui, L., Danzl, N., Campbell, S. R., Viola, R., Williams, D., Xing, Y., Wang, Y., Phillips, N., Poffenberger, G., Johannesson, B., et al. (2018). beta-Cell Replacement in Mice Using Human Type 1 Diabetes Nuclear Transfer Embryonic Stem Cells. Diabetes 67, 26-35.
46. Teo, A. K., Windmueller, R., Johansson, B. B., Dirice, E., Njolstad, P. R., Tjora, E., Raeder, H., and Kulkarni, R. N. (2013). Derivation of human induced pluripotent stem cells from patients with maturity onset diabetes of the young. The Journal of biological chemistry 288, 5353-5356.
47. Tomei, A. A., Villa, C., and Ricordi, C. (2015). Development of an encapsulated stem cell-based therapy for diabetes. Expert Opin Biol Ther 15, 1321-1336.
48. Totsuka, Y., Tabuchi, M., Kojima, I., Eto, Y., Shibai, H., and Ogata, E. (1989). Stimulation of insulin secretion by transforming growth factor-beta. Biochem Biophys Res Commun 158, 1060-1065.
49. Tritschler, S., Theis, F. J., Lickert, H., and Bottcher, A. (2017). Systematic single-cell analysis provides new insights into heterogeneity and plasticity of the pancreas. Molecular metabolism 6, 974-990.
50. Vegas, A. J., Veiseh, O., Gurtler, M., Millman, J. R., Pagliuca, F. W., Bader, A. R., Doloff, J. C., Li, J., Chen, M., Olejnik, K., et al. (2016). Long-term glycemic control using polymer-encapsulated human stem cell-derived beta cells in immune-competent mice. Nat Med 22, 306-311.
51. Weir, G. C., Cavelti-Weder, C., and Bonner-Weir, S. (2011). Stem cell approaches for diabetes: towards beta cell replacement. Genome Med 3, 61.
52. Xin, Y., Kim, J., Okamoto, H., Ni, M., Wei, Y., Adler, C., Murphy, A. J., Yancopoulos, G. D., Lin, C., and Gromada, J. (2016). RNA Sequencing of Single Human Islet Cells Reveals Type 2 Diabetes Genes. Cell metabolism 24, 608-615.
53. Zeng, H., Guo, M., Zhou, T., Tan, L., Chong, C. N., Zhang, T., Dong, X., Xiang, J. Z., Yu, A. S., Yue, L., et al. (2016). An Isogenic Human ESC Platform for Functional Evaluation of Genome-wide-Association-Study-Identified Diabetes Genes and Drug Discovery. Cell stem cell 19, 326-340.
54. Zhang, C. Y., Baffy, G., Perret, P., Krauss, S., Peroni, O., Grujic, D., Hagen, T., Vidal-Puig, A. J., Boss, O., Kim, Y. B., et al. (2001). Uncoupling protein-2 negatively regulates insulin secretion and is a major link between obesity, beta cell dysfunction, and type 2 diabetes. Cell 105, 745-755.
55. Zhu, S., Russ, H. A., Wang, X., Zhang, M., Ma, T., Xu, T., Tang, S., Hebrok, M., and Ding, S. (2016). Human pancreatic beta-like cells converted from fibroblasts. Nat Commun 7, 10080.

### Example 2: Cytoskeletal Regulation of Human Pancreatic Cell Fate

The following example describes cytoskeletal modulation to enhance pancreatic differentiation. The method of cytoskeletal modulation can be used to generate cells of several lineages, not just pancreatic cells. Furthermore, this example describes the methodology for making insulin-producing beta-like cells from human pluripotent stem cells (hPSC) for Type 1 diabetic (T1 D) cell replacement therapy and disease modeling for drug screening.

Recent progress has been made in the differentiation of human pluripotent stem cells (hPSCs) to insulin-producing β cells, with the ultimate goal of a cell replacement therapy for insulin-dependent diabetes. These approaches utilize the addition of soluble factors to activate developmental signal transduction pathways to drive a pancreatic fate. Interestingly, all successful protocols currently must include three-dimensional cell aggregation, but the reasons for this requirement are unknown. This work establishes a link between the microenvironment and the state of the actin cytoskeleton with the expression of crucial pancreatic transcription factors that drive pancreatic lineage specification. The results demonstrate that temporal control of the actin cytoskeleton strongly influences cell fate choice to endodermal lineages. A combination of cell-biomaterial interactions and the actin depolymerizer latrunculin A was used to develop a new two-dimensional differentiation protocol for generating stem cell-derived β (SC-β) cells with a high degree of reproducibility across several hPSC lines that are capable of robust dynamic glucose-stimulated insulin secretion. Furthermore, this work demonstrates that these SC-β cells are capable of rapidly reversing severe pre-existing diabetes in mice.

### Introduction

The recent development of protocols for the production of SC-β cells has offered the promise of a cell-based therapy for the treatment of diabetes. These differentiation strategies rely on the precise activation and repression of specific developmental pathways with soluble growth factors and small molecules to achieve a functional SC-β cell fate. Interestingly, all successful SC-β cell protocols currently must utilize a three-dimensional arrangement of cells either as suspension clusters or as aggregates on an air-liquid interface for the differentiation of pancreatic progenitors to SC-β cells. The reason for this requirement has been unknown, particularly in understanding the effects of the insoluble microenvironment on pancreatic fate choice.

Current methodologies to generate SC-β cells differentiate hPSCs through intermediate endodermal and pancreatic progenitor stages. Given appropriate signals, these progenitors are capable of producing non-pancreatic lineages, such as intestine or hepatocytes (liver cells). Within the pancreatic lineage, premature induction of endocrine genes, such as NEUROG3, before the induction of NKX6-1+ pancreatic progenitors results in the generation of non-β cell polyhormonal cells. While full differentiation to a SC-β cell fate has only been achieved with three-dimensional cell arrangements, induction of this NKX6-1+ phenotype has been demonstrated both in two-dimensional and three-dimensional cell culture.

Cells can sense their surrounding microenvironment via transmembrane proteins called integrins, and the different combinations of the α and β integrin subunits dictate the extracellular matrix (ECM) proteins to which a particular cell can adhere. Integrins bound to ECM proteins cluster together and recruit other adhesion proteins that act as an anchor for the assembly of the actin cytoskeleton, providing a means for cells to generate mechanical forces. Not only do these forces allow cells to migrate and change shape, but they can also be transduced into biochemical signaling within the cell. Specific material properties of the ECM substrate can drastically influence this response by altering the degree of actin polymerization. For example, matrix stiffness, geometry, and adhesion density have all been shown to guide stem cell differentiation. This concept of manipulating the cytoskeleton, however, has not been widely applied to the differentiation of endodermal lineages.

Herein, this work identifies that the state of the actin cytoskeleton is critical to endodermal cell fate choice. In the context of SC-β cells, cytoskeletal state drastically influences NEUROG3-induced endocrine induction and subsequent SC-β cell specification. By utilizing a combination of cell-biomaterial interactions as well as small molecule regulators of the actin cytoskeleton, the timing of endocrine transcription factor expression was controlled to modulate differentiation fate and develop a two-dimensional protocol for making SC-β cells. Importantly, this new planar protocol greatly enhances the function of SC-β cells differentiated from induced pluripotent stem cell (iPSC) lines and forgoes the requirement for three-dimensional cellular arrangements. Different degrees of actin polymerization at specific points of differentiation biased cells toward different endodermal lineages, and thus non-optimal cytoskeletal states led to large inefficiencies in SC-β cell specification. Furthermore, this work demonstrates that this concept of controlling actin polymerization can be applied to directed differentiations of these other endodermal cell fates to modulate lineage specification.

Results

The Actin Cytoskeleton Regulates Maintenance of PDX1-Expressing Progenitors

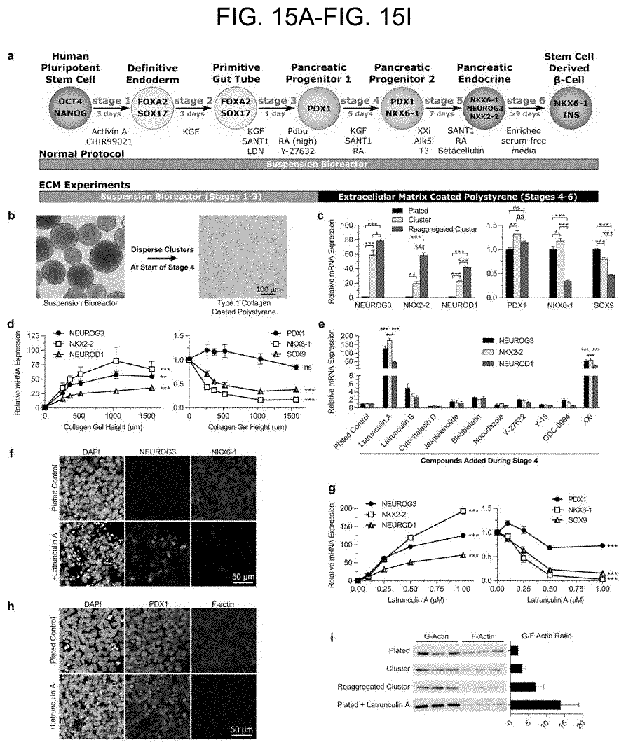

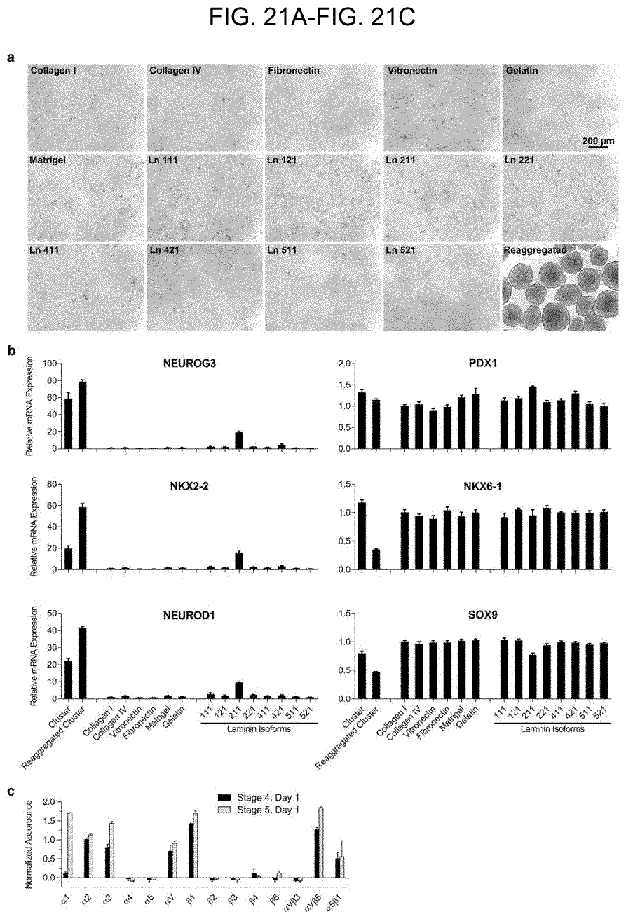

To better understand the role of the microenvironment on SC-β cell differentiation, stage 3 PDX1+ pancreatic progenitor cells were generated with a suspension-based differentiation protocol, a single-cell dispersion was created from these clusters, and cells were seeded onto tissue-culture polystyrene (TCP) plates coated with a wide variety of ECM proteins (see e.g., FIG. 15a-FIG. 15b, FIG. 21a). This stage of the protocol is designed to generate NKX6-1+ pancreatic progenitors, while the subsequent stage 5 initiates endocrine induction of these progenitors by inducing NEUROG3. The most striking observation from these experiments was that plating the cells down for the duration of stage 4 on most ECM proteins prevented the premature expression of NEUROG3 relative to the normal suspension clusters, while reaggregating the cells back into clusters after single-cell dispersion greatly increased expression (see e.g., FIG. 15c, FIG. 21b). Downstream NEUROG3 targets NKX2.2 and NEUROD1 followed the same decreasing trend, while SOX9 expression increased (see e.g., FIG. 15c, FIG. 21b). Interestingly, the ECM protein inducing the highest NEUROG3 expression was laminin 211, which corresponded to poor cell adhesion (see e.g., FIG. 21b). A colorimetric antibody-based integrin adhesion assay at the beginning and end of stage 4 confirmed high expression of integrin subunits that bind to collagens I and IV (α1, α2, β1), fibronectin (αV, β1, α5β1), vitronectin (αV, β1, αVβ5) and some but not all laminin isoforms (α3, β1) (see e.g., FIG. 21c). Thus, strong attachment to the culture surface rather than the composition of a particular ECM protein coating prevented premature endocrine induction during stage 4.

One major difference between culturing cells in suspension as clusters compared to plating them onto TCP plates is the large difference in substrate stiffness experienced by each cell. To test the influence of substrate stiffness on endocrine induction, PDX1-expressing pancreatic progenitors were plated onto type 1 collagen gels of various heights attached to TCP plates, as decreasing gel height increases the effective stiffness experienced by the cell. Increasing gel height led to increases in NEUROG3, NKX2.2, and NEUROD1 and decreases in SOX9, consistent with endocrine induction (see e.g., FIG. 15d). NKX6-1 expression followed the reverse trend as NEUROG3, illustrating that premature NEUROG3 expression induced by a soft substrate is detrimental to NKX6-1 induction in pancreatic progenitors.

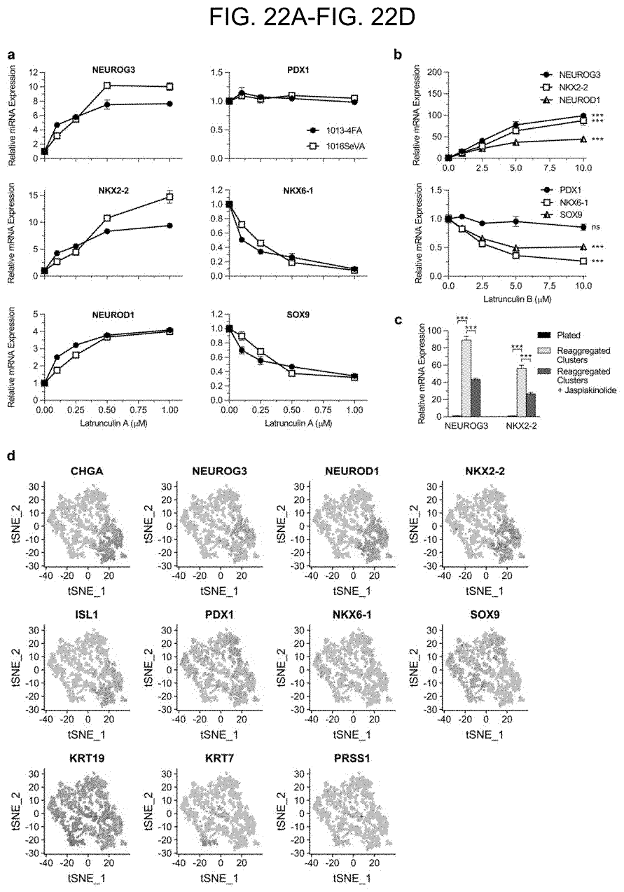

To further probe how cell adhesion affects endocrine induction, a compound screen was performed with factors that influenced different aspects of cellular adhesion. This screen revealed that latrunculin A, which binds and sequesters the monomer form of cytoskeletal actin, greatly increased expression of NEUROG3 as well as its downstream targets NKX2.2 and NEUROD1 (see e.g., FIG. 15e-FIG. 15f). This increase was even larger than that induced by the γ-secretase inhibitor XXi, which inhibits NOTCH signaling and has been used to generate endocrine cells. NEUROG3 expression in response to latrunculin A treatment was highly dose-dependent for both HUES8 (see e.g., FIG. 15g) and two iPSC lines (see e.g., FIG. 22a). Latrunculin B, which is a less potent form of the compound, increased NEUROG3 expression in a dose-dependent manner as well but required ˜10× higher concentration to achieve a similar effect (see e.g., FIG. 22b). NKX6-1 expression followed the reverse trend as NEUROG3 (see e.g., FIG. 15f-FIG. 15g), again illustrating the need to prevent premature NEUROG3 expression in order for NKX6-1 to turn on during stage 4.

Treatment with 1 μM latrunculin A for 24 hours of plated stage 4 cells resulted in almost complete depolymerization of F-actin (see e.g., FIG. 15h) and an increased G/F-actin ratio (see e.g., FIG. 15i), corresponding to high NEUROG3 expression. Furthermore, the G/F-actin ratio for all conditions matched the trend observed for NEUROG3 expression (see e.g., FIG. 15c), with the plated cells having the lowest levels, followed by the normal suspension culture, reaggregated clusters, and finally the plated cells receiving the latrunculin A treatment. In contrast, adding the actin polymerizer jasplakinolide to pancreatic progenitors during reaggregation after dispersion attenuated premature NEUROG3 expression (see e.g., FIG. 22c). Collectively, these data indicate that the polymerization state of the actin cytoskeleton is crucial to the expression of the important pancreatic transcription factors NEUROG3 and NKX6-1.

Cytoskeletal State Guides the Pancreatic Progenitor Program

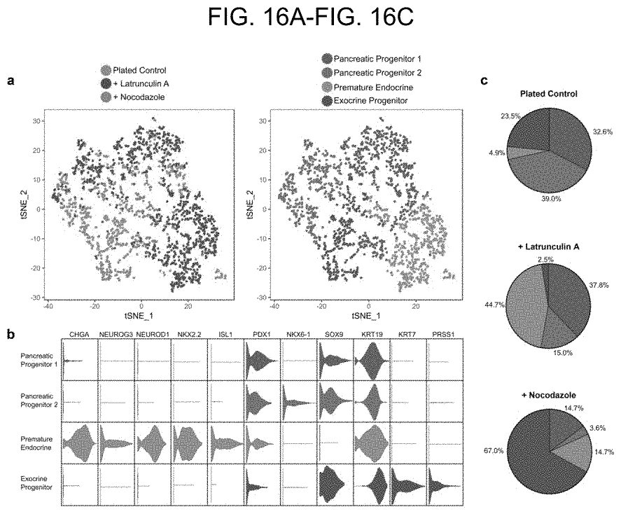

To further investigate how the state of the cytoskeleton influences the pancreatic progenitor program, single-cell RNA sequencing was performed on plated pancreatic progenitors treated with the cytoskeletal-modulating compounds latrunculin A or nocodazole throughout stage 4. While latrunculin A depolymerizes F-actin of these plated progenitors, treatment with nocodazole depolymerizes microtubules, leading to hyper-contraction of F-actin. By the end stage 4, four populations were identified with unsupervised clustering (see e.g., FIG. 16a-FIG. 16b, FIG. 22d). Two populations of pancreatic progenitors were identified by their expression of SOX9 and PDX1 but distinguished based on differential NKX6-1 expression. In contrast, cells experiencing premature endocrine induction had high expression of markers such as CHGA, NEUROG3, NKX2-2, NEUROD1, and ISL1. Importantly, however, they lacked NKX6-1 expression. Exocrine progenitors were characterized by high expression of ductal markers KRT7 and KRT19 and the acinar marker PRSS1 (trypsin). The state of the cytoskeleton during stage 4 had drastic effects on the distribution of cells into these four groups (see e.g., FIG. 16c). The largest population of cells in the plated control (39.0%) were pancreatic progenitor 2 cells that expressed NKX6-1, which is the progenitor population desired at this stage of the protocol. Very few of these plated cells expressed endocrine genes (4.9%). Conversely, latrunculin A treatment decreased the NKX6-1+ population (2.5%) while simultaneously drastically increasing endocrine induction (44.7%). These results correspond to the preceding qRT-PCR data illustrating that plating pancreatic progenitors prevents NEUROG3 from turning on but promotes NKX6-1 expression, while latrunculin A is a potent endocrine inducer. In contrast, treatment with nocodazole promoted exocrine-like progenitors (67.0%). These data suggest that an optimal cytoskeletal state is needed for NKX6-1 expression during stage 4. Specifically, a depolymerized cytoskeleton during stage 4 leads to endocrine induction before NKX6-1 can turn on, while a hyper-activated cytoskeleton also prevents NXK6-1 expression and instead promotes an exocrine progenitor-like fate. Taken together, these data demonstrate that the polymerization state of the actin cytoskeleton in pancreatic progenitors is a crucial regulator of pancreatic cell fate.

Differentiation to SC-β Cells is Temporally Regulated by the Actin Cytoskeleton

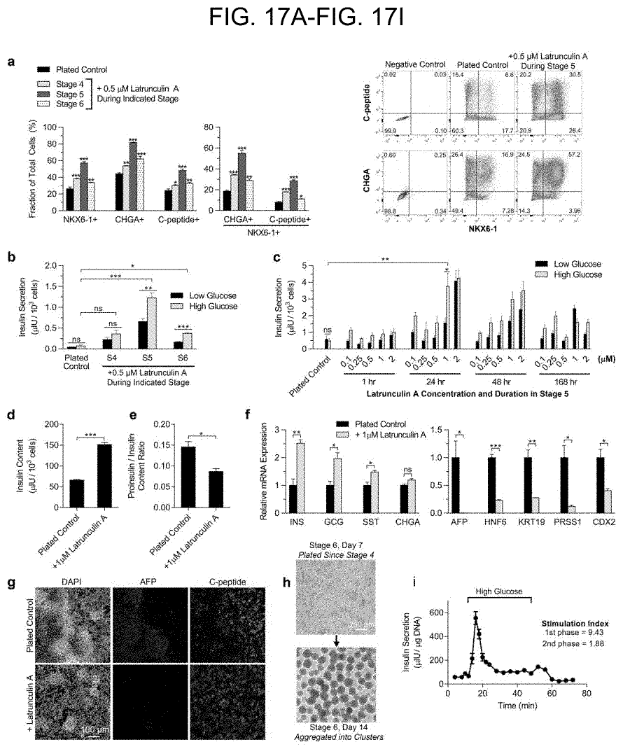

The timing of pancreatic transcription factor expression, notably NKX6-1 and NEUROG3, is critical to proper SC-β cell differentiation. Specifically, non-functional polyhormonal cells or glucagon-positive cells arise if NEUROG3 is expressed before NKX6-1, while NEUROG3 expression after NKX6-1 induction leads to a SC-β cell fate. Because the state of the cytoskeleton was crucial to the expression of these genes, latrunculin A was added throughout different stages of the SC-β cell differentiation protocol after pancreatic progenitors were plated on type 1 collagen-coated TCP. Without the addition of latrunculin A, plated pancreatic progenitors had poor differentiation efficiency (see e.g., FIG. 17a), and the resulting cells secreted little insulin (see e.g., FIG. 17b). Adding 0.5 μM latrunculin A throughout either stage 4 (pancreatic progenitors) or stage 6 (SC-β cell maturation) increased both general endocrine induction (CHGA+) and β-cell specification (NKX6-1+/c-peptide+). However, latrunculin A added during stage 5, which is designed to induce endocrine, led to the greatest increase in endocrine induction, SC-β cell specification, and glucose-stimulated insulin secretion (GSIS) (see e.g., FIG. 17a-b). These data demonstrate that attachment of pancreatic progenitors onto TCP inhibits SC-β cell differentiation, which is overcome by stage-dependent depolymerization of the actin cytoskeleton with latrunculin A.

To optimize the benefits of latrunculin A on SC-β cell induction, a range of durations and concentrations were tested during stage 5 (see e.g., FIG. 17c). Both duration and concentration influenced GSIS, with a 1 μM treatment during the first 24 hours of stage 5 having the most benefit at the shortest and lowest dose. This 24 hour treatment seemed to be sufficient to rescue SC-β cell specification, while extended culture with latrunculin A in stage 5 hampered this effect. Subsequent characterization illustrated that this 24 hour 1 μM latrunculin A treatment increased total insulin content (see e.g., FIG. 17d), improved pro-insulin/insulin ratio (see e.g., FIG. 17e), and increased expression of endocrine genes (see e.g., FIG. 17f). Expression of markers associated with other endodermal lineages was reduced (see e.g., FIG. 17f), as were regions of off-target cell types that were easily distinguished visually by differences in cell morphology and that stained with other non-pancreatic markers, such as AFP (see e.g., FIG. 17g). While plated SC-β cells generated with latrunculin A treatment were functional on TCP in stage 6 (see e.g., FIG. 17c), they could also be aggregated into clusters within 6-well plates on an orbital shaker (see e.g., FIG. 17h). The resulting clusters could be assessed by a dynamic GSIS assay in a perifusion system, exhibiting both first and second phase insulin secretion (see e.g., FIG. 17i).

Collectively, these data demonstrate that the state of the cytoskeleton is critical for maintaining pancreatic progenitors and specifying pancreatic cell fate, particularly to SC-β cells. Specifically, adequate cytoskeletal polymerization is important for the pancreatic progenitor program during stage 4, but differentiation towards SC-β cells requires actin depolymerization during stage 5 endocrine induction. While the high stiffness of TCP induces actin polymerization that prevents premature NEUROG3 expression and promotes NKX6-1 expression during stage 4, it also inhibits NEUROG3 expression during stage 5 and subsequently blocks SC-β cell specification. Treatment with latrunculin A depolymerizes the cytoskeleton during stage 5, enabling robust generation of functional SC-β cells on TCP without the requirement for a three-dimensional cell arrangement.

Latrunculin a Treatment Enables a Planar Protocol for Making SC-β Cells

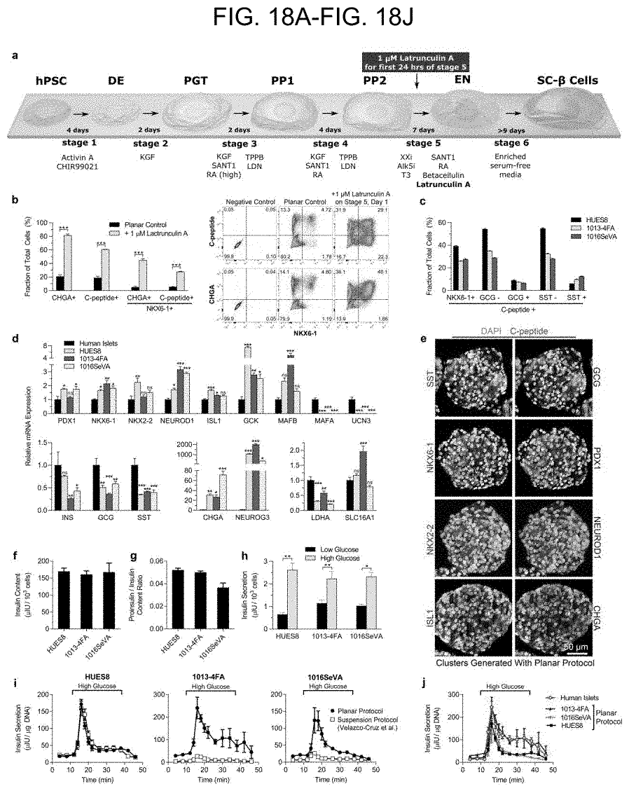

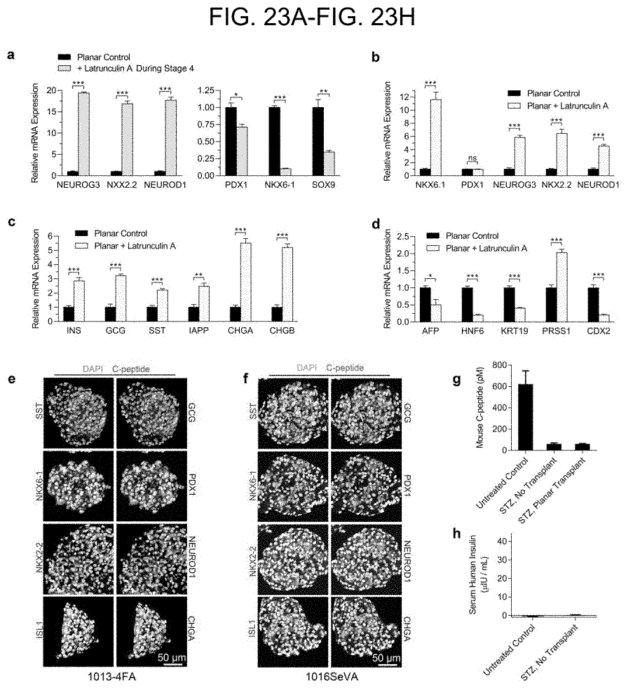

The previous ECM and cytoskeletal experiments initially differentiated cells using the suspension-based differentiation protocol for the first 3 stages to produce pancreatic progenitors followed by attachment onto TCP for continued differentiation and experimentation (see e.g., FIG. 15a). Using the new understanding of the role of the cytoskeleton in pancreatic differentiation, this work developed a new completely planar SC-β cell differentiation protocol to overcome the current requirement in the field of three-dimensional cell arrangements (see e.g., FIG. 18a). Similar to earlier experiments, adding latrunculin A during stage 4 dramatically increased premature expression of NEUROG3 and its downstream targets while simultaneously decreasing NKX6-1 expression (see e.g., FIG. 23a), confirming that pancreatic progenitors generated with both protocols have similar responses to latrunculin A. Without the use of latrunculin A in planar culture, almost no SC-β cells could be generated (see e.g., FIG. 18b), consistent with the requirement of three-dimensional culture in prior reports. However, addition of 1 μM latrunculin A for the first 24 hours of stage 5 during planar differentiation greatly increased endocrine induction and SC-β cell specification while decreasing off-target lineages (see e.g., FIG. 18b, FIG. 22b-FIG. 22d).

To further characterize this new planar differentiation protocol, three hPSC lines from a previous work (HUES8, 1013-4FA, and 1016SeVA) were differentiated with this planar protocol. After one week in stage 6, cells could be aggregated into clusters on an orbital shaker to be used for the same in vitro and in vivo assessment methods as suspension-based differentiations. This yielded aggregated clusters with up to approximately 40% SC-β cells (NKX6-1+/c-peptide+) and low percentages of polyhormonal cells (C-peptide+/GCG+ or C-peptide+/SST+) (see e.g., FIG. 18c). Expression of many β cell and islet genes were similar to the expression in human islets, but MAFA and UCN3 expression remained low (see e.g., FIG. 18d), similar to reports with the suspension protocol. Most cells within these clusters immunostained with c-peptide and were co-positive with several important β-cell markers (see e.g., FIG. 18e, FIG. 23e-FIG. 23f). All three lines had similar insulin content (see e.g., FIG. 18f), pro-insulin/insulin ratio (see e.g., FIG. 18g), static GSIS (see e.g., FIG. 18h), and dynamic GSIS (see e.g., FIG. 18i). Much weaker dynamic function with SC-β cells generated from 1013-4FA and 1016SeVA has been previously reported compared to HUES8 using a suspension-based protocol.⁵ Differentiation with this new planar protocol, however, greatly enhanced both first and second phase dynamic insulin release of these iPSC lines, with dynamic function of all three lines now approaching that of human islets (see e.g., FIG. 18i-FIG. 18j). This planar protocol thus enables greater translatability of SC-β cells generated from different genetic backgrounds.

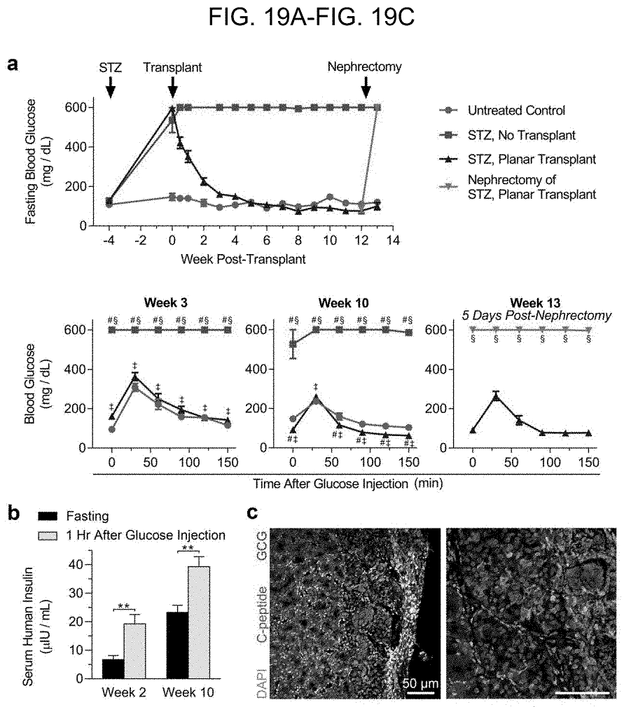

To evaluate in vivo function of these cells, stage 6 clusters generated from HUES8 with the planar protocol were transplanted underneath the kidney capsule of streptozotocin (STZ)-induced diabetic mice (see e.g., FIG. 23g). Fasting glucose levels began approaching those of the untreated controls within two weeks after transplantation, staying below 200 mg/dL afterwards (see e.g., FIG. 19a). Glucose tolerance tests performed at 3 and 10 weeks demonstrated that STZ-treated mice receiving the SC-β cell transplants had similar glucose tolerance as untreated control mice (see e.g., FIG. 19a). Furthermore, high levels of human insulin were detected in the serum of the transplanted mice and were regulated by glucose levels (see e.g., FIG. 19b, FIG. 23h). During week 12 after transplantation, a nephrectomy was performed on 4 transplanted mice to remove the human grafts, resulting in rapid loss of glycemic control and confirming that the restoration of glucose homeostasis arose from the transplanted cells (see e.g., FIG. 19a). Immunostaining of excised kidneys revealed large regions of C-peptide+ cells, and no overgrowths were observed (see e.g., FIG. 19d). Collectively, these data demonstrate that this new planar differentiation protocol generates functional SC-β cells capable of rapidly reversing pre-existing diabetes in mice.

Cytoskeletal Modulation Influences Endodermal Fate Choice

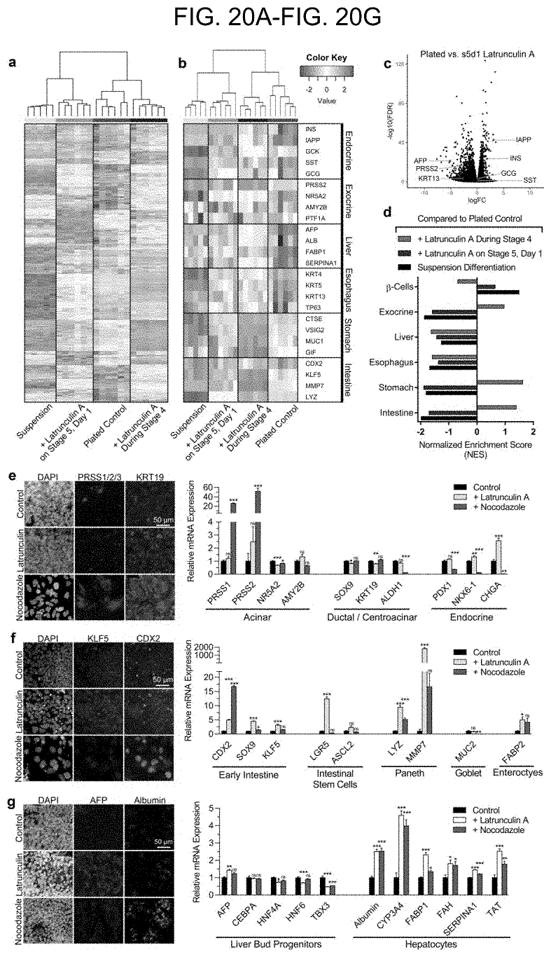

To further investigate the effects of the cytoskeleton on endodermal cell fate choice, a bulk RNA sequencing was performed at stage 6 of the SC-β cell protocol on cells that had been plated during stage 4 and which were treated with latrunculin A during either the pancreatic progenitor stage (stage 4) or during endocrine induction (stage 5). These cells were also compared with untreated plated and suspension differentiations. A heat map of the 1000 most differentially expressed genes illustrates that the timing of latrunculin A treatment had a drastic effect on the expression profile of the resulting cells (see e.g., FIG. 20a). Specifically, the optimal stage 5 latrunculin A treatment shifted the gene expression profile of plated cells toward that of the suspension-based SC-β cell differentiation, increasing expression of β cell and islet genes. Interestingly, many other differentially expressed genes were associated with non-endocrine lineages (see e.g., FIG. 20b-FIG. 20d), with stage 4 latrunculin A treatment increasing intestine and stomach gene expression and the plated control increasing expression of genes associated with the liver and esophagus. Thus, the timing of cytoskeletal modulation is crucial to endodermal cell fate, as having an intact or depolymerized cytoskeleton at specific time points alters endodermal lineage specification.

Collectively, these data indicate that the state of the cytoskeleton is important not only to β cell specification but broadly to endodermal cell fate. Because cytoskeletal modulation influenced fate choice to several endodermal lineages within the SC-β cell protocol, incorporating latrunculin A and nocodazole into other established differentiation protocols was tested for generating exocrine pancreas, intestine, and liver. With the exocrine differentiation, nocodazole greatly increased trypsin gene expression (PRSS1, PRSS2) and immunostaining but inhibited endocrine induction (see e.g., FIG. 20e), corresponding to our earlier single cell RNA sequencing results which indicated nocodazole was driving an exocrine progenitor program. Nocodazole in the intestinal differentiation, on other hand, greatly increased CDX2 gene expression and immunostaining (see e.g., FIG. 20f). Latrunculin A treatment, in contrast, greatly increased markers intestinal stem cells as well as Paneth cells, which are known to be important for LGRS+ intestinal stem cell viability. With the liver differentiation, interestingly, both nocodazole and latrunculin A increased hepatocyte gene expression (see e.g., FIG. 20g). However, immunostaining for albumin was more abundant with nocodazole treatment while AFP was more prevalent with latrunculin A treatment, suggesting differences in hepatic phenotype. As a whole, these data provide a proof-of-principle that the cytoskeleton is a critical component of endodermal cell fate decisions during directed differentiation. While these protocols could certainly benefit from further optimization as this work has demonstrated with the SC-β cell differentiation, these data indicate that the use of specific cytoskeletal-modulating compounds may help increase differentiation efficiency of other endodermal differentiation protocols when used at the appropriate time and dosage. Furthermore, due to the influence that a substrate can have on cytoskeletal dynamics, this data further suggests that culture format is most likely critical to the success of these directed differentiations.

### Discussion

Herein, this work has identified the actin cytoskeleton as a crucial regulator of human pancreatic cell fate. By controlling the state of the cytoskeleton with either cell arrangement (two- vs three-dimensional), substrate stiffness, or directly with chemical treatment, this work has shown that a polymerized cytoskeleton prevents premature induction of NEUROG3 expression in pancreatic progenitors but also inhibits subsequent differentiation to SC-β cells. Appropriately timed cytoskeletal depolymerization with latrunculin A overcomes this inhibition to enable robust generation of SC-β cells. This work has translated these findings to develop a new planar differentiation protocol capable of generating highly functional SC-β cells that undergo first and second phase dynamic insulin secretion and rapidly reverse pre-existing diabetes upon transplantation into mice. Single-cell and bulk RNA sequencing revealed that multiple endodermal lineages, not just SC-β cells, were influenced by the state of the cytoskeleton, and the methods allowed for enhance differentiation to exocrine, intestine, and liver cell fates by cytoskeletal modulation.

There are several distinct advantages for a planar protocol for making SC-β cells, including better control over important transcription factors like NEUROG3 as well as improved cell line reproducibility due to the more controlled, homogenous microenvironment of a tissue culture plate compared to a large cluster of cells. Perhaps the most important benefit of this new protocol, however, is the large improvements in dynamic function of SC-β cells from the two iPSC lines. It has been previously published that with a suspension-based protocol, SC-β cells produced with these two lines have considerably weaker dynamic function. Translatability of differentiation strategies is a longstanding challenge faced by the field and has been particularly problematic when studying patient-derived iPSCs that often have weak in vitro and in vivo SC-β cell phenotypes. Furthermore, it has been observed that certain iPSC lines can often be difficult to even adapt to suspension culture. The use of this planar approach with human patient iPSCs better facilitates rigorous study of diabetes for drug screening and autologous cell replacement therapy for diabetes.

This study also solves a longstanding mystery in the field of why three-dimensional cell arrangements were required for generation of SC-β cells. This study highlights the importance of cell culture format in the study of stem cell differentiation and provides other practical benefits to the SC-β cell field, namely elimination of complicated, laborious, and expensive three-dimensional cell culture requirements. Modulating the cytoskeleton via planar culture and subsequent latrunculin A treatment may also better facilitate the correct timing of NKX6-1 and NEUROG3 expression that promotes functional, monohormonal SC-β cells. The seemingly short time requirement for cytoskeletal depolymerization at the start of endocrine induction is likely due to a positive feedback loop that maintains NEUROG3 expression once it has been turned on. These findings also appear to parallel actin dynamics in vivo, whereby the cytoskeleton is reorganized within cells of the developing pancreatic ducts to induce delamination and subsequent islet formation.

Another important observation from this work is that cytoskeletal state not only regulates SC-β cell differentiation but more broadly influences endodermal lineage specification. Depending on the timing of latrunculin A treatment during SC-β cell differentiations, gene signatures of exocrine, liver, esophagus, stomach, and intestine were detected in stage 6. These findings were applied by adding cytoskeletal-modulating compounds during directed differentiation protocols for some of these other lineages, often improving differentiation outcomes. Thus, the effects of cytoskeletal state are dependent upon the desired endodermal lineage as well as the type and timing of cytoskeletal modulation within these directed differentiation protocols. While these modulations within these other protocols could be further optimized, this work as a whole emphasizes that cytoskeletal dynamics are crucial to endodermal cell fate, with cytoskeletal signaling working synergistically with soluble biochemical factors to regulate cell fate decisions. Consequently, combinations of cell-biomaterial interactions and cytoskeletal-modulating compounds can be leveraged to improve differentiation outcomes to endodermal lineages.

Methods

Stem Cell Culture

Three stem cell lines previously used in SC-β cell differentiation protocols were utilized in this study, including the HUES8 hESC line and two non-diabetic human iPSC lines (1013-4FA and 1016SeVA). Experiments were performed with the HUES8 line unless indicated otherwise. Undifferentiated cells were propagated with mTeSR1 (StemCell Technologies, 05850) in a humidified incubator at 5% CO₂ at 37° C. For suspension culture, cells were passaged every 3 days with Accutase (StemCell Technologies, 07920) and seeded at 0.6×10⁶ cells/mL in 30 mL spinner flasks (REPROCELL, ABBWVS03A) at 60 RPM on a magnetic stir plate (Chemglass). For planar culture, cells were passaged every 4 days with TrypLE (Life Technologies, 12-604-039) and seeded onto Matrigel (Corning, 356230) coated 6-well plates at 3-5 million cells/well, with density dependent on cell line. All cells were seeded in mTeSR1 supplemented with 10 μM Y-27632.

SC-β Cell Differentiation

Suspension protocol: 72 hours after passaging, cells in 30 mL spinner flasks were differentiated in a 6 stage protocol, using the following formulations. Stage 1 (3 days): S1 media+100 ng/ml Activin A (R&D Systems, 338-AC)+3 μM CHIR99021 (Stemgent, 04-0004-10) for 1 day. S1 media+100 ng/ml Activin A for the next 2 days. Stage 2 (3 days): S2 media+50 ng/ml KGF (Peprotech, AF-100-19). Stage 3 (1 day): S3 media+50 ng/ml KGF+200 nM LDN193189 (Reprocell, 040074)+500 nM PdBU (MilliporeSigma, 524390)+2 μM retinoic acid (MilliporeSigma, R2625)+0.25 μM SANT1 (MilliporeSigma, S4572)+10 μM Y27632. Stage 4 (5 days): S3 media+5 ng/mL Activin A+50 ng/mL KGF+0.1 μM retinoic acid+0.25 μM SANT1+10 μM Y27632. Stage 5 (7 days): S5 media+10 μM ALK5i II (Enzo Life Sciences, ALX-270-445-M005)+20 ng/mL Betacellulin (R&D Systems, 261-CE-050)+0.1 μM retinoic acid+0.25 μM SANT1+1 μM T3 (Biosciences, 64245)+1 μM XXI (MilliporeSigma, 595790). Stage 6 (7-25 days): Enriched serum-free media (ESFM). On the first day of stage 6, clusters were resized by single-cell dispersing with TrypLE and reaggregating in a 6-well plate on an orbital shaker (Benchmark Scientific, OrbiShaker) at 100 RPM in ESFM.

The base differentiation media formulations used in each stage were as follows. S1 media: 500 mL MCDB 131 (Cellgro, 15-100-CV) supplemented with 0.22 g glucose (MilliporeSigma, G7528), 1.23 g sodium bicarbonate (MilliporeSigma, S3817), 10 g bovine serum albumin (BSA) (Proliant, 68700), 10 μL ITS-X (Invitrogen, 51500056), 5 mL GlutaMAX (Invitrogen, 35050079), 22 mg vitamin C (MilliporeSigma, A4544), and 5 mL penicillin/streptomycin (P/S) solution (Cellgro, 30-002-CI). S2 media: 500 mL MCDB 131 supplemented with 0.22 g glucose, 0.615 g sodium bicarbonate, 10 g BSA, 10 μL ITS-X, 5 mL GlutaMAX, 22 mg vitamin C, and 5 mL P/S. S3 media: 500 mL MCDB 131 supplemented with 0.22 g glucose, 0.615 g sodium bicarbonate, 10 g BSA, 2.5 mL ITS-X, 5 mL GlutaMAX, 22 mg vitamin C, and 5 mL P/S. S5 media: 500 mL MCDB 131 supplemented with 1.8 g glucose, 0.877 g sodium bicarbonate, 10 g BSA, 2.5 mL ITS-X, 5 mL GlutaMAX, 22 mg vitamin C, 5 mL P/S, and 5 mg heparin (MilliporeSigma, A4544). ESFM: 500 mL MCDB 131 supplemented with 0.23 g glucose, 10.5 g BSA, 5.2 mL GlutaMAX, 5.2 mL P/S, 5 mg heparin, 5.2 mL MEM nonessential amino acids (Corning, 20-025-CI), 84 μg ZnSO₄ (MilliporeSigma, 10883), 523 μL Trace Elements A (Corning, 25-021-CI), and 523 μL Trace Elements B (Corning, 25-022-CI).

For experiments investigating the effects of plating pancreatic progenitors, cells were differentiated with the suspension protocol for stages 1-3. At the end of stage 3, clusters were single cell dispersed with TrypLE and plated onto tissue culture plates coated with various ECM proteins at 0.625×10⁶ cells/cm². Differentiation media for the remainder of this hybrid protocol were the same as for the suspension protocol with the exception that Y-27632 and Activin A were omitted on days 2-5 of stage 4. Additional compounds were added as indicated in each experiment: 1 μM latrunculin A (Cayman Chemical, 10010630), 1 μM latrunculin B (Cayman Chemical, 10010631), 1 μM cytochalasin D (MilliporeSigma, C2618), 1 μM jasplakinolide (Cayman Chemical, 11705), 10 μM blebbistatin (MilliporeSigma, 203389), 1 μM nocodazole (Cayman Chemical, 13857), 1 μM Y-15 (Cayman Chemical, 14485), 10 μM Y-27632, and 10 μM GDC-0994 (Selleckchem, S7554). A variety of ECM coatings were initially tested with this plating methodology, including collagen I (Corning, 354249), collagen IV (Corning, 354245), fibronectin (Gibco, 33016-015), vitronectin (Gibco, A14700), matrigel (Corning, 356230), gelatin (Fisher, G7-500), and laminins 111, 121, 211, 221, 411, 421, 511, and 521 (Biolamina, LNKT-0201). All subsequent experiments with this hybrid protocol were performed on collagen I.

Planar protocol: 24 hours after passaging, cells seeded onto 6 or 24-well plates at 0.313-0.521×10⁶ cells/cm² were differentiated with a new 6 stage protocol using the following formulations, with media changes every day. Stage 1 (4 days): BE1 media+100 ng/mL Activin A+3 μM CHIR99021 for the first 24 hours, followed with 3 days of BE1 containing 100 ng/mL Activin A only. Stage 2 (2 days): BE2 media+50 ng/mL KGF. Stage 3 (2 days): BE3+50 ng/mL KGF, 200 nM LDN193189, 500 nM TPPB (Tocris, 53431), 2 μM retinoic acid, and 0.25 μM SANT1. Stage 4 (4 days): BE3+50 ng/mL KGF, 200 nM LDN193189, 500 nM TPPB, 0.1 μM retinoic acid, and 0.25 μM SANT1. Stage 5 (7 days): S5 media+10 μM ALK5i II+20 ng/mL Betacellulin+0.1 μM retinoic acid+0.25 μM SANT1+1 μM T3+1 μM XXI. 1 μM Latrunculin A was added to this media for the first 24 hours only. Stage 6 (7-25 days): Cultures were kept on the plate with ESFM for the first 7 days. To move to suspension culture, cells could be single cell dispersed with TrypLE and placed in 6 mL ESFM within a 6-well plate at a concentration of 4-5 million cells/well on an orbital shaker at 100 RPM. Assessments were performed 5-8 days after cluster aggregation.

The base differentiation media formulations that differed from the suspension protocol were as follows. BE1 media: 500 mL MCDB 131 supplemented with 0.8 g glucose, 0.587 g sodium bicarbonate, 0.5 g BSA, and 5 mL GlutaMAX. BE2 media: 500 mL MCDB 131 supplemented with 0.4 g glucose, 0.587 g sodium bicarbonate, 0.5 g BSA, 5 mL GlutaMAX, and 22 mg vitamin C. BE3 media: 500 mL MCDB 131 supplemented with 0.22 g glucose, 0.877 g sodium bicarbonate, 10 g BSA, 2.5 mL ITS-X, 5 mL GlutaMAX, and 22 mg vitamin C.

Microscopy and Immunocytochemistry

Brightfield images were taken with a Leica DMi1 inverted light microscope, and fluorescence images were captured with a Nikon A1Rsi confocal microscope. For immunostaining, cells were fixed with 4% paraformaldehyde (PFA) at room temperature for 30 minutes. They were then blocked and permeabilized for 45 minutes at room temperature with an immunocytochemistry (ICC) solution consisting of 0.1% triton X (Acros Organics, 327371000) and 5% donkey serum (Jackson Immunoresearch, 017000-121) in PBS (Corning, 21-040-CV). Samples were then incubated with primary antibodies diluted in ICC solution overnight at 4° C., washed with ICC, incubated with secondary antibodies diluted in ICC for 2 hours at room temperature, and stained with DAPI for 15 minutes at room temperature. For histological sectioning, whole SC-β cell clusters generated with the planar protocol and mouse kidneys containing transplanted cells were fixed overnight with 4% PFA at 4° C. The in vitro clusters were also embedded in Histogel (Thermo Scientific, hg-4000-012). These samples were then paraffin-embedded and sectioned by the Division of Comparative Medicine (DCM) Research Animal Diagnostic Laboratory Core at Washington University in St. Louis. Paraffin was removed from sectioned samples with Histoclear (Thermo Scientific, C78-2-G), and antigen retrieval was carried out in a pressure cooker (Proteogenix, 2100 Retriever) with 0.05 M EDTA (Ambion, AM9261). Slides were blocked and permeabilized with ICC solution for 45 minutes, incubated with primary antibodies in ICC solution overnight at 4° C., and incubated with secondary antibodies for 2 hours at room temperature. Slides were then sealed with DAPI Fluoromount-G (SouthernBiotech, 0100-20).

Primary antibodies were diluted in ICC solution at 1:300 unless indicated otherwise: rat anti-C-peptide (DSHB, GN-ID4-S), 1:100 mouse anti-NKX6-1 (DSHB, F55A12-S), goat anti-PDX1 (R&D Systems, AF2419), sheep anti-NEUROG3 (R&D Systems, AF2746), 1:200 TRITC-conjugated phalloidin (MilliporeSigma, FAK 100), rabbit anti-somatostatin (ABCAM, ab64053), mouse anti-glucagon (ABCAM, ab82270), mouse anti-NKX2-2 (DSHB, 74.5A5-S), goat anti-NEUROD1 (R&D Systems, AF2746), mouse anti-ISL1 (DSHB, 40.2d6-s), rabbit anti-CHGA (ABCAM, ab15160), 1:100 sheep anti-PRSS1/2/3 (R&D Systems, AF3586), 1:100 mouse-anti-KRT19 (Dako, M0888), goat anti-KLF5 (R&D Systems, AF3758), rabbit anti-CDX2 (Abcam, ab76541), mouse anti-AFP (Abcam, ab3980), rabbit anti-albumin (Abcam, ab207327).

Secondary antibodies were diluted in ICC solution at 1:300. All secondary antibodies were raised in donkey: anti-goat alexa fluor 594 (Invitrogen, A11058), anti-goat alexa fluor 647 (Invitrogen, A31571, anti-mouse alexa fluor 488 (Invitrogen, A21202), anti-mouse alexa fluor 594 (Invitrogen, A21203), anti-mouse alexa fluor 647 (Invitrogen, A31571), anti-rabbit alexa fluor 488 (Invitrogen, A21206), anti-rabbit alexa fluor 594 (Invitrogen, A21207), anti-rabbit alexa fluor 647 (Invitrogen, A31573), anti-rat alexa fluor 488 (Invitrogen, A21208), anti-sheep alexa fluor 594 (Invitrogen, A11016).

qRT-PCR

RNA was extracted from either whole clusters or cells directly on the plate with the RNeasy Mini Kit (Qiagen, 74016). Samples were treated with a DNAse kit (Qiagen, 79254) during extraction. The High Capacity cDNA Reverse Transcriptase Kit (Applied Biosystems, 4368814) was used to synthesize cDNA on a thermocycler (Applied Biosystems, A37028). The PowerUp SYBR Green Master Mix (Applied Biosystems, A25741) was used on a StepOnePlus (Applied Biosystems), and real time PCR results were analyzed using a ΔΔCt methodology. TBP and GUSB were both used as housekeeping genes. Primer sequences were as follows.

TABLE 2
Primer sequences for qRT-PCR.

| | SEQ | | SEQ | |
|---|---|---|---|---|
| | ID | Forward primer | ID | Reverse primer |
| Gene name | NO. | sequence | NO. | sequence |
| TBP | 9 | GCCATAAGGCATCATT | 10 | AACAACAGCCTGCCAC |
| | | GGAC | | CTTA |
| GUSB | 23 | CGTCCCACCTAGAATC | 24 | TTGCTCACAAAGGTCA |
| | | TGCT | | CAGG |
| INS | 1 | CAATGCCACGCTTCTG | 2 | TTCTACACACCCAAGA |
| | | C | | CCCG |
| CHGA | 13 | TGACCTCAACGATGCA | 14 | CTGTCCTGGCTCTTCT |
| | | TTTC | | GCTC |
| NEUROD1 | 15 | ATGCCCGGAACTTTTT | 16 | CATAGAGAACGTGGCA |
| | | CTTT | | GCAA |
| SST | 7 | TGGGTTCAGACAGCAG | 8 | CCCAGACTCCGTCAGT |
| | | CTC | | TTCT |
| GCG | 5 | AGCTGCCTTGTACCAG | 6 | TGCTCTCTCTTCACCT |
| | | CATT | | GCTCT |
| PDX1 | 3 | CGTCCGCTTGTTCTCC | 4 | CCTTTCCCATGGATGA |
| | | TC | | AGTC |
| NKX2-2 | 19 | GGAGCTTGAGTCCTGA | 20 | TCTACGACAGCAGCGA |
| | | GGG | | CAAC |
| NKX6-1 | 11 | CCGAGTCCTGCTTCTT | 12 | ATTCGTTGGGGATGAC |
| | | CTTG | | AGAG |
| ISL1 | 39 | TCACGAAGTCGTTCTT | 40 | CATGCTTTGTTAGGGA |
| | | GCTG | | TGGG |
| GCK | 29 | ATGCTGGACGACAGAG | 30 | CCTTCTTCAGGTCCTC |
| | | CC | | CTCC |
| MAFB | 31 | CATAGAGAACGTGGCA | 32 | ATGCCCGGAACTTTTT |
| | | GCAA | | CTTT |
| AFP | 41 | TGTACTGCAGAGATAA | 42 | CCTTGTAAGTGGCTTC |
| | | GTTTAGCTGAC | | TTGAACA |
| PRSS1 | 43 | TATCAGCAGGCCACTG | 44 | CCTCCAGGACTTCGAT |
| | | CTAC | | GTTG |
| CDX2 | 45 | GAACCTGTGCGAGTGG | 46 | TAAGCCTGGGGCTCAA |
| | | ATG | | ACT |
| SOX2 | 47 | TTGCTGCCTCTTTAAG | 48 | GGTCAGTAACCTCGGA |
| | | ACTAGGA | | CCTG |
| KRT19 | 49 | AGGATGCTGAAGCCTG | 50 | GGTCAGTAACCTCGGA |
| | | GTT | | CCTG |
| SERPINA1 | 51 | CCCTGTTTGCTCCTCC | 52 | GATGCCCCACGAGACA |
| | | GATAA | | GAAG |
| FAH | 53 | GCCAGTGTGCTGGAAA | 54 | CTGGCAGGGAGGCTTT |
| | | AGTG | | ACAC |
| HNF4A | 55 | GGACATGGCCGACTAC | 56 | CTCGAGGCACCGTAGT |
| | | AGTG | | GTTT |
| CEBPA | 57 | TATAGGCTGGGCTTCC | 58 | AGCTTTCTGGTGTGAC |
| | | CCTT | | TCGG |
| CYP3A4 | 59 | CACCCCCAGTTAGCAC | 60 | CCACGCCAACAGTGAT |
| | | CATT | | TACA |
| FABP1 | 61 | TCTCCGGCAAGTACCA | 62 | GATTTCCGACACCCCC |
| | | ACTG | | TTGA |
| LGR5 | 63 | CTTGGTGCCCAAAGCT | 64 | TCTTTTCCAGGTATGT |
| | | CA | | TCATTGC |
| ASCL2 | 65 | CACTGGGGATCTGTGG | 66 | TTCTGTAAGGCCCAAA |
| | | ACTG | | GCGT |
| FABP2 | 67 | GCCCAAGGACAGACCT | 68 | CAAGTGCTGTCAAACG |
| | | GAAT | | CCAT |
| MUC2 | 69 | CAGCTCATCTCGTCCG | 70 | GTGTAGGTGTGTGTCA |
| | | TCTC | | GCGA |
| MMP7 | 71 | CATGATTGGCTTTGCG | 72 | CTACCATCCGTCCAGC |
| | | CGAG | | GTTC |
| LYZ | 73 | TCAGCCTAGCACTCTG | 74 | GCCCTGGACCGTAACA |
| | | ACCT | | GALA |
| PRSS2 | 75 | GCTACAAGTCGGCAAT | 76 | CGATGTTGTGCTCTCC |
| | | TAACTCA | | CAGT |
| AMY2B | 77 | GGAGCCTCTGTGTTTC | 78 | GCACTTGAAGGACACG |
| | | TTTGTT | | GGA |
| NR5A2 | 79 | CCGACAAGTGGTACAT | 80 | TCCGGCTTGTGATGCT |
| | | GGAA | | ATTA |
| ALDH1 | 81 | ATCAAAGAAGCTGCCG | 82 | GCATTGTCCAAGTCGG |
| | | GGAA | | CATC |
| TAT | 83 | CAGTCCCCGAGGTGAT | 84 | CTGAGTGTGGGTGTGG |
| | | GATG | | TTGT |
| TBX3 | 85 | AAACTCTGCGCGGAGA | 86 | CCCCCAGTAGCTCAAT |
| | | AAGA | | GCAA |
| HNF6 | 87 | ATGTCCAGCGTCGAAC | 88 | TGCTTTGGTACAAGTG |
| | | TCTAC | | CTTGAT |
| LDHA | 33 | GGAGATCCATCATCTC | 34 | GGCCTGTGCCATCAGT |
| | | TCCC | | ATCT |
| SLC16A1 | 37 | CACTTAAAATGCCACC | 38 | AGAGAAGCCGATGGAA |
| | | AGCA | | ATGA |
| MAFA | 27 | GAGAGCGAGAAGTGCC | 28 | TTCTCCTTGTACAGGT |
| | | AACT | | CCCG |
| UCN3 | 25 | GGAGGGAAGTCCACTC | 26 | TGTAGAACTTGTGGGG |
| | | TCG | | GAGG |

Collagen Gels

Type 1 collagen (Corning, 354249) gels were created at a concentration of 5 mg/mL using 10×PBS, sterile deionized water, and 1 M NaOH according to the manufacturer's instructions. Various volumes of this collagen solution were pipetted into the center of wells of a 24-well plate and briefly centrifuged to obtain a uniform coating. Collagen gel heights were calculated based on the volume of collagen gel solution, the radius of the 24-well plate, and the equation for the height of a cylinder.

G/F Actin Ratio

G/F actin ratio was determined by western blot following the instructions of the G-actin/F-actin In Vivo Assay Kit (Cytoskeleton, Inc, BK037). Western blot was visualized using SuperSignal West Pico PLUS Chemiluminescent substrate (ThermoScientific, 34577) and the Odyssey FC (LI-COR) imager.

Integrin Assay

To quantify which integrins were expressed on the surface of pancreatic progenitors, cells generated in suspension culture were dispersed with TrypLE at either the end of stage 3 or stage 4 and plated onto wells coated with monoclonal antibodies for different a and 13 integrin subunits using the Alpha/Beta Integrin-Mediated Cell Adhesion Array Combo Kit (MilliporeSigma, ECM532). Integrin expression was quantified according to the manufacturer's instructions.

Single-Cell RNA Sequencing

Cells generated with the suspension protocol were single-cell dispersed with TrypLE from clusters at the end of stage 3 and seeded onto collagen 1 coated 24-well plates at 0.625×10⁶ cells/cm². Either 0.5 μM latrunculin A or 5 μM nocodazole were added throughout the entirety of stage 4. At the end of stage 4, cells were single-cell dispersed, suspended in DMEM, and submitted to the Washington University Genome Technology Access Center. Library preparation was done using the Chromium Single Cell 3′ Library and Gel Bead Kit v2 (10× Genomics, 120237). Briefly, single cells were isolated in emulsions using a microfluidic platform, and each single cell emulsion was barcoded with a unique set of oligonucleotides. The GemCode Platform was used to carry out reverse transcription within each single cell emulsion, which was amplified to construct a library. The libraries were sequenced with paired-end reads of 26×98 primerbp using the Illumina HiSeq2500.

Seurat v2.0 was used to perform single cell RNA analyses. Duplicate cells and cells with high mitochondrial gene expression were filtered out using FilterCells (>9000 total genes and >5% mitochondrial genes for Untreated Control, >6000 genes and >6% mitochondrial genes for latrunculin A, >12000 genes and >4% mitochondrial genes for nocodazole). Each data set was normalized using global-scaling normalization. FindVariableGenes identified and removed outlier genes using scaled z-score dispersion. The datasets were then combined and a canonical correlation analysis (CCA) was performed with RunMultiCCA. AlignSubspace was used to align the CCA subspaces and generated a new dimension reduction for integrated analysis. Unsupervised TSNE plots were generated using RunTSNE, and the resulting clusters were defined and labeled using FindMarkers. VInPlot (Violin plots) and FeaturePlot (tsne plots) were used to visualize differences in gene expressions across each cluster and conditions.

Flow Cytometry

Cells were single-cell dispersed with TrypLE and fixed with 4% PFA for 30 minutes. Cells were then washed with PBS and incubated with ICC solution for 45 minutes at room temperature, incubated with primary antibodies overnight at 4° C., and incubated with secondary antibodies for 2 hours at room temperature. Cells were then washed twice with ICC solution and filtered before running on the LSRII flow cytometer (BD Biosciences). Analysis was completed with FlowJo.

Glucose Stimulated Insulin Secretion

Static GSIS: To assess the function of cells produced by the hybrid protocol, static GSIS was performed with cells still attached to 96 or 24-well tissue culture plates. To assess function of clusters generated with the planar protocol, approximately 30 clusters were collected and placed in tissue culture transwell inserts (MilliporeSigma, PIXP01250) in a 24-well plate. All were first washed twice with KRB buffer (128 mM NaCl, 5 mM KCl, 2.7 mM CaCl₂) 1.2 mM MgSO₄, 1 mM Na₂HPO₄, 1.2 mM KH₂PO₄, 5 mM NaHCO₃, 10 mM HEPES (Gibco, 15630-080), and 0.1% BSA). Cells were first incubated in a 2 mM glucose KRB solution at 37° C. for one hour, after which this solution was discarded and replaced with fresh 2 mM glucose KRB. After an additional hour, the supernatant was collected. 20 mM glucose KRB was added for the next hour, after which the supernatant was again collected. Cells were washed with fresh KRB during each solution change. Cells were then single-cell dispersed with TrypLE and counted with the Vi-Cell XR (Beckman Coulter). Supernatants from the low and high glucose challenges were quantified with a human insulin ELISA (ALPCO, 80-INSHU-E10.1), and cell counts were used to normalize insulin secretion.

Dynamic GSIS: Dynamic function of SC-β cells was assessed with a perifusion setup as we have reported.⁵ 0.015 inch inlet and outlet tubing (ISMATEC, 070602-04i-ND) was connected with 0.04″ connection tubing (BioRep, Peri-TUB-040) to 275-μl cell chambers (BioRep, Pen-Chamber) and dispensing nozzles (BioRep, PERI-NOZZLE). Approximately 30 SC-β cell clusters were washed twice with KRB buffer and loaded into the chambers, sandwiched between two layers of hydrated Bio-Gel P-4 polyacrylamide beads (Bio-Rad, 150-4124). These chambers were connected to a high precision 8-channel dispenser pump (ISMATEC, ISM931C) and immersed in a 37° C. water bath for the remainder of the assay. A 2 mM glucose KRB solution was perfused through the chambers for the first 90 minutes at a flow rate of 100 μL/min. After this equilibration period, effluent was collected in 2 minute time intervals, switching glucose solutions as follows: 2 mM glucose KRB for 12 minutes, 20 mM glucose KRB for 24 minutes, and 2 mM glucose KRB for 16 minutes. The SC-β cell clusters were then lysed with a solution of 10 mM Tris (MilliporeSigma, T6066), 1 mM EDTA (Ambion, AM9261), and 0.2% Triton-X (Acros Organics, 327371000). DNA was quantified using the Quant-iTPicogreen dsDNA assay kit (Invitrogen, P7589) and was used to normalize insulin values quantified with a human insulin ELISA.

Insulin and Proinsulin Content

Whole SC-β cell clusters or cells attached to culture plates were washed twice thoroughly with PBS. Half of the clusters or an equivalent well of plated cells were immersed in TrypLE for cell counts on the Vi-Cell XR. For the other half of the samples, a solution of 1.5% HCl and 70% ethanol was added to either the clusters in eppendorf tubes or directly onto plated cells. After 15 minutes, the plated cells were pipetted vigorously and transferred to eppendorf tubes. The eppendorf tubes from both clusters and plated cells were kept at −20° C. for 72 hours, vortexing vigorously every 24 hours. Samples were then centrifuged at 2100 RCF for 15 minutes. The supernatant of each sample was collected, neutralized with an equal volume of 1 M TRIS (pH 7.5), and quantified using proinsulin ELISA (Mercodia, 10-1118-01) and human insulin ELISA kits. Proinsulin and insulin secretion were normalized to the viable cell counts.

Transplantation Studies

In vivo studies were carried out in accordance to the Washington University International Care and Use Committee regulations 0.7-week-old male immunodeficient mice (NOD.Cg-Prkdcscid II2rgtm1Wjl/SzJ) were purchased from Jackson Laboratories. Randomly selected mice were induced with diabetes by administering 45 mg/kg STZ (R&D Systems, 1621500) in PBS for 5 consecutive days via intraperitoneal injection. Mice became diabetic approximately one week after STZ treatment. After 2 more weeks, transplant surgeries were performed by injecting ˜5 million SC-β cells generated with the planar protocol under the kidney capsule of diabetic mice anaesthetized with isoflurane. All mice were monitored weekly after transplant surgeries. Removal of the kidneys containing SC-β cells of randomly selected transplanted mice were performed during week 12 after transplant.

Fasting blood glucose measurements, glucose tolerance tests, and in vivo GSIS were performed for in vivo assessments. Mice were fasted 4-6 hours for all studies. For fasting measurements, blood glucose levels were obtained from a tail bleed using a handheld glucometer (Bayer, 9545C). For glucose tolerance tests, 2 g/kg glucose in 0.9% saline (Moltox, 51-405022.052) were injected and measured blood glucose every 30 minutes for 150 minutes. For in vivo GSIS, approximately 30 μL of blood via tail bleed was collected using microvettes (Sarstedt, 16.443.100) before and 60 minutes post glucose injection. The blood samples were centrifuged at 2500 rpm for 15 minutes at 4° C. and the serum was collected to be quantified with the Human Ultrasensitive Insulin ELISA kit (ALPCO Diagnostics, 80-ENSHUU-E01.1) and Mouse C-peptide ELISA kit (ALPCO Diagnostics, 80-CPTMS-E01).

Bulk RNA Sequencing

Cells generated with the suspension protocol were single-cell dispersed from clusters with TrypLE at the end of stage 3 and seeded onto collagen 1 coated 24-well plates at 0.625×10⁶ cells/cm². Either 0.5 μM latrunculin A was added throughout the entirety of stage 4 or 1 μM latrunculin A was added for the first 24 hours of stage 5. After two weeks in stage 6, RNA was extracted with the RNeasy Mini Kit (Qiagen, 74016), including a DNase treatment (Qiagen, 79254) during extraction. Samples were delivered to Washington University in St. Louis Genome Technology Access Center for library preparation and sequencing. Samples were prepared by RNA depletion using Ribo-Zero according to library kit manufacturer's protocol, indexed, pooled, and sequenced on an Illumine HiSeq.

Differential gene expression analysis was performed using EdgeR. DGEList was used to create the count object and normalized the data using the trimmed mean M-values (TMM) method with calcNormFactors. Pairwise comparisons were performed using exactTest and used topTags to obtain differentially expressed genes and their respective log fold change (log FC) and adjusted p-value (FDR). These values were used to generate volcano plots using ggplot2. Hierarchical clustering and heatmaps were performed and generated with heatmap.2 (gplots) using log CPM calculated expression levels. Gene set analyses were performed with gene set enrichment analysis (GSEA). Lineage specific gene sets including Exocrine (GO: 0035272, M13401), Pancreas Beta cells (Hallmark, M5957) and Intestinal epithelial (GO: 0060576, M12973) were obtained from the Molecular Signatures Database (MdigDB). Gene sets for liver, esophagus and stomach were customized using the Human Protein Atlas and literature.

Differentiation to Other Endodermal Lineages

For differentiation to other endodermal lineages, HUES8 stem cells were cultured and passaged normally. Differentiations were initiated 24 hours after seeding 24-well plates at 0.521×10⁶ cells/cm². Protocols for exocrine pancreas, intestine, and liver were adapted, from literature. Either latrunculin A or nocodazole were added as indicated in each protocol. All three differentiation protocols used the same stage 1 to induce endoderm. Stage 1 (4 days): BE1 media+100 ng/mL Activin A+3 μM CHIR99021 for the first 24 hours, followed with 3 days of BE1 containing 100 ng/mL Activin A only.

Exocrine Pancreas: Stage 2 (2 days): BE2 media+50 ng/mL KGF. Stage 3 (2 days): BE3+50 ng/mL KGF, 200 nM LDN193189, 500 nM TPPB, 2 μM retinoic acid, and 0.25 μM SANT1. Stage 4 (4 days): BE3+50 ng/mL KGF, 200 nM LDN193189, 500 nM TPPB, 0.1 μM retinoic acid, and 0.25 μM SANT1. Either 1 μM latrunculin A was added for the first 24 hours of this stage, or 1 μM nocodazole was added for the entirety of stage 4. Stage 5 (6 days): S5 media+10 ng/mL bFGF. 10 mM nicotinamide (MilliporeSigma, 72340) was added for the last two days.

Intestine Differentiation: Stage 2 (4 days): BE2 media+3 μM CHIR99021+500 ng/mL FGF4 (R&D Systems, 235-F4). Either 1 μM latrunculin A was added for the first 24 hours of this stage, or 1 μM nocodazole was added for the entirety of stage 2. Stage 3 (7 days): BE3 media+500 ng/mL R-spondin1 (R&D Systems, 4645-RS)+100 ng/mL EGF (R&D Systems, 236-EG)+200 nM LDN193189.

Liver Differentiation: Stage 2 (2 days): BE2 media+50 ng/mL KGF. Stage 3 (4 days): BE3 media+10 ng/mL bFGF+30 ng/mL BMP4 (R&D Systems, 314-BP). For the first 24 hours only, 2 μM retinoic acid and either 1 μM latrunculin A or 1 μM nocodazole were added. Stage 4 (5 days): BE3 media+20 ng/mL OSM (R&D Systems, 295-OM)+20 ng/mL HGF (R&D Systems, 294-HG)+100 nM dexamethasone (MilliporeSigma, D4902).

Statistical Analysis

Data analysis was performed in GraphPad Prism, version 7. Analyzed data was evaluated by either two-sided t-tests or ANOVA followed by either Dunnett's multiple comparison test or Tukey's HSD test. The following convention is used for indicating p-values: ns=not significant, *=p<0.05, **=p<0.01, ***=p<0.001. All data error bars represent SEM. The sample size (n) indicates the total number of biological replicates.

### REFERENCES

1. Millman, J. R. & Pagliuca, F. W. Autologous pluripotent stem cell-derived β-like cells for diabetes cellular therapy. Diabetes 66, 1111-1120 (2017).
2. Tomei, A. A., Villa, C. & Ricordi, C. Development of an encapsulated stem cell-based therapy for diabetes. Expert Opin. Biol. Ther. 15, 1321-1336 (2015).
3. Jennings, R. E., Berry, A. A., Strutt, J. P., Gerrard, D. T. & Hanley, N. A. Human pancreas development. Development 142, 3126-3137 (2015).
4. Pagliuca, F. W. et al. Generation of functional human pancreatic β cells in vitro. Cell 159, 428-439 (2014).
5. Velazco-Cruz, L. et al. Acquisition of Dynamic Function in Human Stem Cell-Derived 13 Cells. Stem Cell Reports 12, 351-365 (2019).
6. Rezania, A. et al. Reversal of diabetes with insulin-producing cells derived in vitro from human pluripotent stem cells. Nat. Biotechnol. 32, 1121-1133 (2014).
7. Russ, H. A. et al. Controlled induction of human pancreatic progenitors produces functional beta-like cells in vitro. EMBO J. 34, 1759-1772 (2015).
8. Nair, G. G. et al. Recapitulating endocrine cell clustering in culture promotes maturation of human stem-cell-derived β cells. Nat. Cell Biol. 21, 263-274 (2019).
9. Sui, L. et al. β-Cell replacement in mice using human type 1 diabetes nuclear transfer embryonic stem cells. Diabetes 67, 26-35 (2018).
10. Ghazizadeh, Z. et al. ROCKII inhibition promotes the maturation of human pancreatic beta-like cells. Nat. Commun. 8, (2017).
11. D'Amour, K. A. et al. Efficient differentiation of human embryonic stem cells to definitive endoderm. Nat. Biotechnol. 23, 1534-1541 (2005).
12. Amour, K. A. D. et al. Production of pancreatic hormone-expressing endocrine cells from human embryonic stem cells. 24, 1392-1401 (2006).
13. Kroon, E. et al. Pancreatic endoderm derived from human embryonic stem cells generates glucose-responsive insulin-secreting cells in vivo. Nat. Biotechnol. 26, 443-452 (2008).
14. Nostro, M. C. et al. Efficient generation of NKX6-1+ pancreatic progenitors from multiple human pluripotent stem cell lines. Stem Cell Reports 4, 591-604 (2015).
15. Rezania, A. et al. Maturation of human embryonic stem cell-derived pancreatic progenitors into functional islets capable of treating pre-existing diabetes in mice. Diabetes 61, 2016-2029 (2012).
16. Spence, J. R. et al. Directed differentiation of human pluripotent stem cells into intestinal tissue in vitro. Nature 470, 105-110 (2011).
17. Cheng, X. et al. Self-renewing endodermal progenitor lines generated from human pluripotent stem cells. Cell Stem Cell 10, 371-384 (2012).
18. Gu, G., Dubauskaite, J. & Melton, D. A. Direct evidence for the pancreatic lineage: NGN3+ cells are islet progenitors and are distinct from duct progenitors. Development 129, 2447-57 (2002).
19. Johansson, K. A. et al. Temporal Control of Neurogenin3 Activity in Pancreas Progenitors Reveals Competence Windows for the Generation of Different Endocrine Cell Types. Dev. Cell 12, 457-465 (2007).
20. Nelson, S. B., Schaffer, A. E. & Sander, M. The transcription factors Nkx6.1 and Nkx6.2 possess equivalent activities in promoting beta-cell fate specification in Pdx1+ pancreatic progenitor cells. Development 134, 2491-2500 (2007).
21. Schulz, T. C. et al. A scalable system for production of functional pancreatic progenitors from human embryonic stem cells. PLoS One 7, (2012).
22. Barczyk, M., Carracedo, S. & Gullberg, D. Integrins. Cell Tissue Res. 339, 269-280 (2010).
23. Vicente-Manzanares, M., Ma, X., Adelstein, R. S. & Horwitz, A. R. Non-muscle myosin II takes centre stage in cell adhesion and migration. Nat. Rev. Mol. Cell Biol. 10, 778-90 (2009).
24. Engler, A. J., Sen, S., Sweeney, H. L. & Discher, D. E. Matrix elasticity directs stem cell lineage specification. Cell 126, 677-89 (2006).
25. Hogrebe, N. J. & Gooch, K. J. Direct influence of culture dimensionality on hMSC differentiation at various matrix stiffnesses using a fibrous self-assembling peptide hydrogel. J. Biomed. Mater. Res. Part A 104, 2356-68 (2016).
26. Kilian, K. a, Bugarija, B., Lahn, B. T. & Mrksich, M. Geometric cues for directing the differentiation of mesenchymal stem cells. Proc. Natl. Acad. Sci. U.S.A 107, 4872-7 (2010).
27. McBeath, R., Pirone, D. M., Nelson, C. M., Bhadriraju, K. & Chen, C. S. Cell shape, cytoskeletal tension, and RhoA regulate stem cell lineage commitment. Dev. Cell 6, 483-95 (2004).
28. Ahn, E. H. et al. Spatial control of adult stem cell fate using nanotopographic cues. Biomaterials 35, 2401-10 (2014).
29. Frith, J. E., Mills, R. J. & Cooper-White, J. J. Lateral spacing of adhesion peptides influences human mesenchymal stem cell behaviour. J. Cell Sci. 125, 317-27 (2012).
30. Hogrebe, N. J. et al. Independent control of matrix adhesiveness and stiffness within a 3D self-assembling peptide hydrogel. Acta Biomater. 70, 110-119 (2018).
31. Leong, W. S. et al. Thickness sensing of hMSCs on collagen gel directs stem cell fate. Biochem. Biophys. Res. Commun. 401, 287-92 (2010).
32. Brenner, S. L. & Korn, D. inhibition of actin polymerization by latrunculin A. FEBS Lett. 2, 316-318 (1987).
33. Peng, G. E., Wilson, S. R. & Weiner, 0. D. A pharmacological cocktail for arresting actin dynamics in living cells. Mol. Biol. Cell 22, 3986-3994 (2011).
34. GEF-H1 Couples Nocodazole-induced Microtubule Disassembly to Cell Contractility via RhoA. Mol. Biol. Cell 19, 2147-2153 (2008).
35. Hohwieler, M. et al. Human pluripotent stem cell-derived acinar/ductal organoids generate human pancreas upon orthotopic transplantation and allow disease modelling. Gut 66, 473-486 (2017).
36. McCracken, K. W., Howell, J. C., Wells, J. M. & Spence, J. R. Generating human intestinal tissue from pluripotent stem cells in vitro. Nat. Protoc. 6, 1920-1928 (2011).
37. Ang, L. T. et al. A Roadmap for Human Liver Differentiation from Pluripotent Stem Cells Resource A Roadmap for Human Liver Differentiation from Pluripotent Stem Cells. Cell Rep. 22, 2190-2205 (2018).
38. Yin, X. et al. Niche-independent high-purity cultures of Lgr5+ intestinal stem cells and their progeny. Nat. Methods 11, 106-112 (2014).
39. Wang, X. et al. Point mutations in the PDX1 transactivation domain impair human β-cell development and function. Mol. Metab. 1-18 (2019). doi:10.1016/J.MOLMET.2019.03.006
40. Balboa, D. et al. Insulin mutations impair beta-cell development in a patient-derived iPSC model of neonatal diabetes. Elife 7, 1-35 (2018).
41. Ma, S. et al. β Cell Replacement after Gene Editing of a Neonatal Diabetes-Causing Mutation at the Insulin Locus. Stem Cell Reports 11, 1407-1415 (2018).
42. Maxwell, K. et al. Rapid reversal of pre-existing diabetes in mice with transplantation of CRISPR/Cas9-corrected human stem cell-derived diabetic patient β cells. Submitted (2019).
43. Seymour, P. A. Sox9: A master regulator of the pancreatic program. Rev. Diabet. Stud. 11, 51-83 (2014).
44. Kesavan, G. et al. Cdc42/N-WASP signaling links actin dynamics to pancreatic cell delamination and differentiation. J. Cell Sci. 127, e1-e1 (2014).
45. Butler, A., Hoffman, P., Smibert, P., Papalexi, E. & Satija, R. Integrating single-cell transcriptomic data across different conditions, technologies, and species. Nat. Biotechnol. 36, 411-420 (2018).
46. McCarthy, D. J., Chen, Y. & Smyth, G. K. Differential expression analysis of multifactor RNA-Seq experiments with respect to biological variation. Nucleic Acids Res. 40, 4288-4297 (2012).
47. Robinson, M. D., McCarthy, D. J. & Smyth, G. K. edgeR: A Bioconductor package for differential expression analysis of digital gene expression data. Bioinformatics 26, 139-140 (2009).
48. Subramanian, A. et al. Gene set enrichment analysis: a knowledge-based approach for interpreting genome-wide expression profiles. Proc. Natl. Acad. Sci. U.S.A 102, 15545-50 (2005).
49. Mootha, V. K. et al. PGC-1α-responsive genes involved in oxidative phosphorylation are coordinately downregulated in human diabetes. 34, 1-7 (2003).
50. Liberzon, A. et al. Molecular signatures database (MSigDB) 3.0. Bioinformatics 27, 1739-1740 (2011).
51. Liberzon, A. et al. The Molecular Signatures Database Hallmark Gene Set Collection. Cell Syst. 1, 417-425 (2015).
52. Uhlén, M. et al. Tissue-based map of the human proteome. Science. 347, 1-9 (2015).
53. Takebe, T. et al. Vascularized and functional human liver from an iPSC-derived organ bud transplant. Nature 499, 481-484 (2013).
54. Finkbeiner, S. R. et al. Transcriptome-wide Analysis Reveals Hallmarks of Human Intestine Development and Maturation In Vitro and In Vivo. Stem Cell Reports 4, 1140-1155 (2015).
55. McCracken, K. W. et al. Modelling human development and disease in pluripotent stem-cell-derived gastric organoids. Nature 516, 400-404 (2014).
56. Rosekrans, S. L., Baan, B., Muncan, V. & van den Brink, G. R. Esophageal development and epithelial homeostasis. Am. J. Physiol. Liver Physiol. 309, G216-G228 (2015).
57. Trisno, S. L. et al. Esophageal Organoids from Human Pluripotent Stem Cells Delineate Sox2 Functions during Esophageal Specification. Cell Stem Cell 23, 501-515.e7 (2018).
58. Willet, S. G. & Mills, J. C. Stomach Organ and Cell Lineage Differentiation: From Embryogenesis to Adult Homeostasis. Cmgh 2, 546-559 (2016).

1. A method of generating insulin-producing beta cells in a suspension comprising:
providing a stem cell;
providing serum-free media; and
contacting the stem cell with a TGFβ/Activin agonist or a glycogen synthase kinase 3 (GSK) inhibitor or WNT agonist for an amount of time sufficient to form a definitive endoderm cell;
contacting the definitive endoderm cell with a FGFR2b agonist for an amount of time sufficient to form a primitive gut tube cell;
contacting the primitive gut tube cell with an RAR agonist, and optionally a rho kinase inhibitor, a smoothened antagonist, a FGFR2b agonist, a protein kinase C activator, or a BMP type 1 receptor inhibitor for an amount of time sufficient to form an early pancreas progenitor cell;
incubating the early pancreas progenitor cell for at least about 3 days and optionally contacting the early pancreas progenitor cell with a rho kinase inhibitor, a TGF-β/Activin agonist, a smoothened antagonist, an FGFR2b agonist, or a RAR agonist for an amount of time sufficient to form a pancreatic progenitor cell; or
contacting the pancreatic progenitor cell with an Alk5 inhibitor, a gamma secretase inhibitor, SANT1, Erbb1 (EGFR) or Erbb4 agonist, or a RAR agonist for an amount of time sufficient to form an endoderm cell; and
reducing cell cluster size comprising resizing the cell clusters (optionally within about 24 hours of incubation) and allowing the endoderm cell to mature in serum-free media for an amount of time sufficient to form a beta cell.

2. The method of claim 1, wherein
the TGFβ/Activin agonist is Activin A;
the glycogen synthase kinase 3 (GSK) inhibitor or the WNT agonist is CHIR;
the FGFR2b agonist is KGF;
the smoothened antagonist is SANT-1;
the RAR agonist is retinoic acid (RA);
the protein kinase C activator is PdBU;
the BMP type 1 receptor inhibitor is LDN;
the rho kinase inhibitor is Y27632;
the Alk5 inhibitor is Alk5i; or
the Erbb4 agonist is betacellulin.

3. The method of claim 1, wherein the serum-free media comprises one or more selected from the group consisting of:
MCDB131, glucose, NaHCO₃, BSA, ITS-X, Glutamax, vitamin C, penicillin-streptomycin, CMRL 10666, FBS, Heparin, NEAA, trace elements A, trace elements B, or ZnSO₄.

4. The method of claim 1, comprising reducing cluster size of the endoderm, wherein resizing cell clusters comprise breaking apart clusters and reaggregating prior to maturation into beta cells.

5. The method of claim 1, wherein the pancreatic progenitor cell is not incubated with any one or more of serum, T3, N-acetyl cysteine, Trolox, and R428.

6. The method of claim 1, wherein the amount of time sufficient to form a definitive endoderm cell, a primitive gut tube cell, an early pancreas progenitor cell, a pancreatic progenitor cell, an endoderm cell is between about 1 day and about 8 days or the amount of time sufficient to form a beta cell in between about 1 day and about 9 days or more than 9 days.

7. The method of claim 1, wherein the method does not comprise the use of a TGFβR1 inhibitor (optionally, Alk5 inhibitor II) or thyroid hormone (optionally, T3) in the maturation of endoderm cells to beta cells.

8. The method of claim 7, wherein the absence of a TGFβR1 inhibitor allows for TGFβ signaling and promotes functional maturation of endoderm cells to beta cells or allows for an increased cell insulin secretion in response to an increased glucose level or an increased secretogouge level.

9. The method of claim 7, wherein the method does not comprise T3, N-acetyl cysteine, Trolox, or R428 in the maturation of endoderm cells to beta cells.

10. The method of claim 1, wherein
the beta cell is an SC-β cell expressing at least one β cell marker, at least one islet cell marker, and undergoes glucose-stimulated insulin secretion (GSIS) comprising first and second phase dynamic insulin secretion;
the beta cell secretes insulin in substantially similar amounts compared to cadaveric human islets; or
the beta cell retains functionality for 1 or more days.

11. The method of claim 1, wherein the stem cell is an induced pluripotent stem cell (iPSC) (such as a patient-derived iPSC), an HUES8 embryonic cell, 1013-4FA, SEVA 1016, or SEVA 1019.

12. (canceled)

13. A method of differentiating a stem cell into a cell of endodermal lineage comprising:
providing a stem cell;
providing serum-free media; and
contacting the stem cell with a TGFβ/Activin agonist and a glycogen synthase kinase 3 (GSK) inhibitor or WNT agonist for an amount of time sufficient to form a definitive endoderm cell;
contacting the definitive endoderm cell with a FGFR2b agonist for an amount of time sufficient to form a primitive gut tube cell;
contacting the primitive gut tube cell with an RAR agonist and, optionally, a smoothened antagonist/sonic hedgehog inhibitor, a FGF family member/FGFR2b agonist, a protein kinase 3 activator, a BMP inhibitor, or a rho kinase inhibitor, optionally, for an amount of time sufficient to form an early pancreas progenitor cell;
incubating the early pancreas progenitor cell for at least about 3 days and optionally comprising contacting the early pancreas progenitor cell with a smoothened antagonist, an FGFR2b agonist, a RAR agonist, a rho kinase inhibitor, or a TGF-β/Activin agonist, for an amount of time sufficient to form a pancreatic progenitor cell;
contacting the pancreatic progenitor cell with an Alk5 inhibitor/TGF-β receptor inhibitor, thyroid hormone, and a gamma secretase inhibitor and optionally SANT1, a Erbb1 (EGFR) or Erbb4 agonist/EGF family member, or a RAR agonist for an amount of time sufficient to form an endodermal cell or endocrine cell;
optionally contacting the endodermal cell or the endocrine cell with an Alk5 inhibitor/TGF-β receptor inhibitor or a thyroid hormone for an amount of time sufficient to form a cell of endodermal lineage (e.g., pancreatic cell, liver cell, or beta cell/SC-β cell); and
modulating the cytoskeleton comprising plating cells on a stiff (such as a tissue culture plastic (TCP) with a layer of ECM protein to promote attachment) or soft substrate or introducing a cytoskeletal-modulating agent to cells, optionally the cytoskeletal-modulating agent comprises latrunculin A, latrunculin B, nocodazole, cytochalasin D, jasplakinolide, blebbistatin, y-27632, y-15, gdc-0994, or an integrin modulating agent, at a time and for an amount of time sufficient to increase differentiation efficiency.

14. A method of differentiating a stem cell into a cell of endodermal lineage comprising:
incubating a stem cell in media comprising a TGFβ/Activin agonist, Activin A, a WNT agonist, and CHIR for about 24 hours, followed by about 3 days of incubating cells in media comprising the Activin A absent CHIR, resulting in stage 1, definitive endoderm cells; and
generating exocrine pancreas cells comprising incubating the stage 1, definitive endoderm cells for about two days in media comprising a FGFR2b agonist, KGF, resulting in stage 2 cells; incubating the stage 2 cells for 2 days in media comprising the FGFR2b agonist, KGF; a BMP inhibitor, LDN193189; TPPB; a RAR agonist, retinoic acid (RA); and a smoothened antagonist, SANT1, resulting in stage 3 cells; incubating stage 3 cells for about four days in media comprising the FGFR2b agonist, KGF; the BMP inhibitor, LDN193189; TPPB; the RAR agonist, retinoic acid; and the smoothened antagonist, SANT1, resulting in stage 4 cells, wherein latrunculin A is added for about the first 24 hours of incubation or nocodazole is added for an entirety of about four days of incubation; and incubating stage 4 cells in media comprising bFGF for about six days, wherein nicotinamide is added during the last two days of the six days;
generating intestine cells comprising incubating the stage 1, definitive endoderm cells for about four days in media comprising the WNT agonist, CHIR and FGF4, wherein latrunculin A is added for about the first 24 hours of incubation or nocodazole is added for the entirety of about four days of incubation, resulting in stage 2 cells;
incubating stage 2 cells for about 7 days in media comprising R-spondin1 and the BMP inhibitor, LDN193189; or
generating liver cells comprising incubating the stage 1, definitive endoderm cells for about two days in media comprising the FGFR2b agonist, KGF, resulting in stage 3 cells; incubating stage 3 cells for about four days in media comprising BMP4, wherein the RAR agonist, retinoic acid and either latrunculin A or nocodazole were added for about the first 24 to 48 hours of incubation, resulting in stage 4 cells; and incubating the stage 4 cells in media comprising OSM, HGF, and dexamethasone for about 5 days.

15. The method of claim 13 or 14, comprising resizing clusters prior to forming a cell of endodermal lineage.

16. The method of claim 13, wherein
the TGFβ/Activin agonist is Activin A;
the glycogen synthase kinase 3 (GSK) inhibitor or the WNT agonist is CHIR;
the FGFR2b agonist is KGF;
the smoothened antagonist or sonic hedgehog inhibitor is SANT-1;
the FGF family member/FGFR2b agonist is KGF;
the RAR agonist is RA;
the protein kinase 3 activator is PDBU;
the BMP inhibitor is LDN;
the rho kinase inhibitor is Y27632;
the Alk5 inhibitor/TGF-β receptor inhibitor is Alk5i;
the thyroid hormone is T3;
the gamma secretase inhibitor is XXI;
the Erbb1 (EGFR) or Erbb4 agonist/EGF family member is betacellulin; or
RAR agonist is RA.

17. The method of any one of claim 13, wherein the media is serum-free media comprises one or more selected from the group consisting of: MCDB131, glucose, NaHCO₃, BSA, ITS-X, Glutamax, vitamin C, penicillin-streptomycin, CMRL 10666, FBS, Heparin, NEAA, trace elements A, trace elements B, or ZnSO₄.

18. The method of claim 13, wherein the amount of time sufficient to form a definitive endoderm cell, a primitive gut tube cell, an early pancreas progenitor cell, a pancreatic progenitor cell, an endoderm cell, or a beta cell is between about 1 day and about 15 days.

19. The method of claim 13, wherein the early pancreatic progenitor cells are plated or YAP activated with s1p (sphingosine-1-phosphate), to increase SC-β cell induction, prevent undesirable premature endocrine commitment, or allow for correct timing of transcription factor expression.


SEQUENCE LISTING (91 sequences)

SEQ ID NO: 1 - DNA, 17 bp, Artificial Sequence
  primer
caatgccacgcttctgc

SEQ ID NO: 2 - DNA, 20 bp, Artificial Sequence
  primer
ttctacacacccaagacccg

SEQ ID NO: 3 - DNA, 18 bp, Artificial Sequence
  prime
cgtccgcttgttctcctc

SEQ ID NO: 4 - DNA, 20 bp, Artificial Sequence
  primer
cctttcccatggatgaagtc

SEQ ID NO: 5 - DNA, 20 bp, Artificial Sequence
  primer
agctgccttgtaccagcatt

SEQ ID NO: 6 - DNA, 21 bp, Artificial Sequence
  primer
tgctctctcttcacctgctct

SEQ ID NO: 7 - DNA, 19 bp, Artificial Sequence
  primer
tgggttcagacagcagctc

SEQ ID NO: 8 - DNA, 20 bp, Artificial Sequence
  primer
cccagactccgtcagtttct

SEQ ID NO: 9 - DNA, 20 bp, Artificial Sequence
  primer
gccataaggcatcattggac

SEQ ID NO: 10 - DNA, 20 bp, Artificial Sequence
  primer
aacaacagcctgccacctta

SEQ ID NO: 11 - DNA, 20 bp, Artificial Sequence
  primer
ccgagtcctgcttcttcttg

SEQ ID NO: 12 - DNA, 20 bp, Artificial Sequence
  primer
attcgttggggatgacagag

SEQ ID NO: 13 - DNA, 20 bp, Artificial Sequence
  primer
tgacctcaacgatgcatttc

SEQ ID NO: 14 - DNA, 20 bp, Artificial Sequence
  primer
ctgtcctggctcttctgctc

SEQ ID NO: 15 - DNA, 20 bp, Artificial Sequence
  primer
atgcccggaactttttcttt

SEQ ID NO: 16 - DNA, 20 bp, Artificial Sequence
  primer
catagagaacgtggcagcaa

SEQ ID NO: 17 - DNA, 19 bp, Artificial Sequence
  primer
cttcgtcttccgaggctct

SEQ ID NO: 18 - DNA, 20 bp, Artificial Sequence
  primer
ctattcttttgcgccggtag

SEQ ID NO: 19 - DNA, 19 bp, Artificial Sequence
  primer
ggagcttgagtcctgaggg

SEQ ID NO: 20 - DNA, 20 bp, Artificial Sequence
  primer
tctacgacagcagcgacaac

SEQ ID NO: 21 - DNA, 21 bp, Artificial Sequence
  primer
cgacggcgttacagtgtttct

SEQ ID NO: 22 - DNA, 21 bp, Artificial Sequence
  primer
cccatctgtcacacaagtaaa

SEQ ID NO: 23 - DNA, 20 bp, Artificial Sequence
  primer
cgtcccacctagaatctgct

SEQ ID NO: 24 - DNA, 20 bp, Artificial Sequence
  primer
ttgctcacaaaggtcacagg

SEQ ID NO: 25 - DNA, 19 bp, Artificial Sequence
  primer
ggagggaagtccactctcg

SEQ ID NO: 26 - DNA, 20 bp, Artificial Sequence
  primer
tgtagaacttgtgggggagg

SEQ ID NO: 27 - DNA, 20 bp, Artificial Sequence
  primer
gagagcgagaagtgccaact

SEQ ID NO: 28 - DNA, 20 bp, Artificial Sequence
  primer
ttctccttgtacaggtcccg

SEQ ID NO: 29 - DNA, 18 bp, Artificial Sequence
  primer
atgctggacgacagagcc

SEQ ID NO: 30 - DNA, 20 bp, Artificial Sequence
  primer
ccttcttcaggtcctcctcc

SEQ ID NO: 31 - DNA, 20 bp, Artificial Sequence
  primer
catagagaacgtggcagcaa

SEQ ID NO: 32 - DNA, 20 bp, Artificial Sequence
  primer
atgcccggaactttttcttt

SEQ ID NO: 33 - DNA, 20 bp, Artificial Sequence
  primer
ggcctgtgccatcagtatct

SEQ ID NO: 34 - DNA, 20 bp, Artificial Sequence
  primer
ggagatccatcatctctccc

SEQ ID NO: 35 - DNA, 17 bp, Artificial Sequence
  primer
atggagcccagcagcaa

SEQ ID NO: 36 - DNA, 21 bp, Artificial Sequence
  primer
ggcattgatgactccagtgtt

SEQ ID NO: 37 - DNA, 20 bp, Artificial Sequence
  primer
cacttaaaatgccaccagca

SEQ ID NO: 38 - DNA, 20 bp, Artificial Sequence
  primer
agagaagccgatggaaatga

SEQ ID NO: 39 - DNA, 20 bp, Artificial Sequence
  primer
tcacgaagtcgttcttgctg

SEQ ID NO: 40 - DNA, 20 bp, Artificial Sequence
  primer
catgctttgttagggatggg

SEQ ID NO: 41 - DNA, 27 bp, Artificial Sequence
  primer
tgtactgcagagataagtttagctgac

SEQ ID NO: 42 - DNA, 23 bp, Artificial Sequence
  primer
ccttgtaagtggcttcttgaaca

SEQ ID NO: 43 - DNA, 20 bp, Artificial Sequence
  primer
tatcagcaggccactgctac

SEQ ID NO: 44 - DNA, 20 bp, Artificial Sequence
  primer
cctccaggacttcgatgttg

SEQ ID NO: 45 - DNA, 19 bp, Artificial Sequence
  primer
gaacctgtgcgagtggatg

SEQ ID NO: 46 - DNA, 19 bp, Artificial Sequence
  primer
taagcctggggctcaaact

SEQ ID NO: 47 - DNA, 23 bp, Artificial Sequence
  primer
ttgctgcctctttaagactagga

SEQ ID NO: 48 - DNA, 20 bp, Artificial Sequence
  primer
ggtcagtaacctcggacctg

SEQ ID NO: 49 - DNA, 19 bp, Artificial Sequence
  primer
aggatgctgaagcctggtt

SEQ ID NO: 50 - DNA, 20 bp, Artificial Sequence
  primer
ggtcagtaacctcggacctg

SEQ ID NO: 51 - DNA, 21 bp, Artificial Sequence
  primer
ccctgtttgctcctccgataa

SEQ ID NO: 52 - DNA, 20 bp, Artificial Sequence
  primer
gatgccccacgagacagaag

SEQ ID NO: 53 - DNA, 20 bp, Artificial Sequence
  primer
gccagtgtgctggaaaagtg

SEQ ID NO: 54 - DNA, 20 bp, Artificial Sequence
  primer
ctggcagggaggctttacac

SEQ ID NO: 55 - DNA, 20 bp, Artificial Sequence
  primer
ggacatggccgactacagtg

SEQ ID NO: 56 - DNA, 20 bp, Artificial Sequence
  primer
ctcgaggcaccgtagtgttt

SEQ ID NO: 57 - DNA, 20 bp, Artificial Sequence
  primer
tataggctgggcttcccctt

SEQ ID NO: 58 - DNA, 20 bp, Artificial Sequence
  primer
agctttctggtgtgactcgg

SEQ ID NO: 59 - DNA, 20 bp, Artificial Sequence
  primer
cacccccagttagcaccatt

SEQ ID NO: 60 - DNA, 20 bp, Artificial Sequence
  primer
ccacgccaacagtgattaca

SEQ ID NO: 61 - DNA, 20 bp, Artificial Sequence
  primer
tctccggcaagtaccaactg

SEQ ID NO: 62 - DNA, 20 bp, Artificial Sequence
  primer
gatttccgacacccccttga

SEQ ID NO: 63 - DNA, 18 bp, Artificial Sequence
  primer
cttggtgcccaaagctca

SEQ ID NO: 64 - DNA, 23 bp, Artificial Sequence
  primer
tcttttccaggtatgttcattgc

SEQ ID NO: 65 - DNA, 20 bp, Artificial Sequence
  primer
cactggggatctgtggactg

SEQ ID NO: 66 - DNA, 20 bp, Artificial Sequence
  primer
ttctgtaaggcccaaagcgt

SEQ ID NO: 67 - DNA, 20 bp, Artificial Sequence
  primer
gcccaaggacagacctgaat

SEQ ID NO: 68 - DNA, 20 bp, Artificial Sequence
  primer
caagtgctgtcaaacgccat

SEQ ID NO: 69 - DNA, 20 bp, Artificial Sequence
  primer
cagctcatctcgtccgtctc

SEQ ID NO: 70 - DNA, 20 bp, Artificial Sequence
  primer
gtgtaggtgtgtgtcagcga

SEQ ID NO: 71 - DNA, 20 bp, Artificial Sequence
  primer
catgattggctttgcgcgag

SEQ ID NO: 72 - DNA, 20 bp, Artificial Sequence
  primer
ctaccatccgtccagcgttc

SEQ ID NO: 73 - DNA, 20 bp, Artificial Sequence
  primer
tcagcctagcactctgacct

SEQ ID NO: 74 - DNA, 20 bp, Artificial Sequence
  primer
gccctggaccgtaacagaaa

SEQ ID NO: 75 - DNA, 23 bp, Artificial Sequence
  Primer
gctacaagtcggcaattaactca

SEQ ID NO: 76 - DNA, 20 bp, Artificial Sequence
  primer
cgatgttgtgctctcccagt

SEQ ID NO: 77 - DNA, 22 bp, Artificial Sequence
  primer
ggagcctctgtgtttctttgtt

SEQ ID NO: 78 - DNA, 19 bp, Artificial Sequence
  primer
gcacttgaaggacacggga

SEQ ID NO: 79 - DNA, 20 bp, Artificial Sequence
  primer
ccgacaagtggtacatggaa

SEQ ID NO: 80 - DNA, 20 bp, Artificial Sequence
  primer
tccggcttgtgatgctatta

SEQ ID NO: 81 - DNA, 20 bp, Artificial Sequence
  primer
atcaaagaagctgccgggaa

SEQ ID NO: 82 - DNA, 20 bp, Artificial Sequence
  primer
gcattgtccaagtcggcatc

SEQ ID NO: 83 - DNA, 20 bp, Artificial Sequence
  primer
cagtccccgaggtgatgatg

SEQ ID NO: 84 - DNA, 20 bp, Artificial Sequence
  primer
ctgagtgtgggtgtggttgt

SEQ ID NO: 85 - DNA, 20 bp, Artificial Sequence
  primer
aaactctgcgcggagaaaga

SEQ ID NO: 86 - DNA, 20 bp, Artificial Sequence
  primer
cccccagtagctcaatgcaa

SEQ ID NO: 87 - DNA, 21 bp, Artificial Sequence
  primer
atgtccagcgtcgaactctac

SEQ ID NO: 88 - DNA, 22 bp, Artificial Sequence
  primer
tgctttggtacaagtgcttgat

SEQ ID NO: 89 - DNA, 21 bp, Artificial Sequence
  shRNA GFP
gcgcgatcacatggtcctgct

SEQ ID NO: 90 - DNA, 21 bp, Artificial Sequence
  shRNA TGFBR1 #1
gatcatgattactgtcgataa

SEQ ID NO: 91 - DNA, 21 bp, Artificial Sequence
  TGFBR1 #2
gcaggattctttaggctttat